(19) United States
(12) Patent Application Publication
(10) Pub. No.: US 2026/0345029 A1
(43) Pub. Date: Nov. 5, 2026

(54) Wearable Wireless Finger Goniometer

(71) Applicants: Washington University, St. Louis, MO (US); Northwestern University - Evanston, Evanston, IL (US)

(72) Inventors: Mitchell Pet, St. Louis, MO (US); John Rogers, Evanston, IL (US); Hee Sup Shin, Evanston, IL (US)

(73) Assignees: Washington University, St. Louis, MO (US); Northwestern University - Evanston, Evanston, IL (US)

(21) Appl. No.: 19/666,174

(22) Filed: May 1, 2026

Related U.S. Application Data
(60) Provisional application No. 63/797,969, filed on May 1, 2025.

(51) Int. Cl. A61B 5/107; A61B 5/00; A61B 90/00

(52) U.S. Cl. CPC A61B 5/1071; A61B 5/1079; A61B 5/6824; A61B 5/6826; A61B 5/6829; A61B 5/6831; A61B 5/6832; A61B 5/7235; A61B 5/742; A61B 5/7475; A61B 90/36; A61B 90/39; A61B 2090/363; A61B 2090/3937; A61B 2560/0214; A61B 2560/0223; A61B 2562/0223; A61B 2562/16; A61B 2562/166

(57) ABSTRACT

Wearable wireless goniometric systems, devices, and associated methods of monitoring joint angle changes are disclosed herein.

### CROSS-REFERENCE TO RELATED APPLICATIONS

This application claims the benefit of priority to U.S. Provisional Application Ser. No. 63/797,969 filed on May 1, 2025, which is incorporated herein by reference in its entirety.

### STATEMENT REGARDING FEDERALLY SPONSORED RESEARCH OR DEVELOPMENT

Not applicable.

### MATERIAL INCORPORATED-BY-REFERENCE

Not applicable.

### FIELD OF THE INVENTION

The present disclosure generally relates to systems and methods for medical imaging texture analysis to predict fetal development and/or maternal health.

### BACKGROUND OF THE INVENTION

Impairment in the upper extremity, including the arm, forearm, wrist, and hand, can significantly reduce independence in activities of daily living as well as quality of life, while also incurring substantial financial costs. Although the causes of such impairments vary-ranging from stroke to spinal injury and trauma-ensuring optimal recovery through proper rehabilitation is crucial; inadequate rehabilitation can lead to impaired recovery, often resulting in a permanent loss of range of motion (ROM) and motor skills. However, fewer than 55% of patients typically comply with their rehabilitation programs, missing the critical window for optimal recovery. Biofeedback that incorporates quantitative measurements of joint kinematics has proven to be a valuable tool for both patients and clinicians during rehabilitation, as it provides real-time biological information that would otherwise be inaccessible. This real-time data can enhance therapeutic outcomes by helping patients participate more effectively with their rehabilitation protocols, in both the supervised and unsupervised settings.

In nearly all traumatic conditions of the hand, injury and treatment are associated with joint stiffness and lost range of motion. Rehabilitation from the injured state, as such, is highly focused upon the restoration of maximal ROM. Clinical assessment of ROM is a fundamental parameter used to evaluate joint movement functionality, mobility, and rehabilitation progress. Standard approaches to measuring hand ROM encompass visual estimation, goniometry, composite finger flexion to the distal palmar crease, torque range of motion, and wiregrams 16. The most ubiquitous tool in clinical hand therapy is the hand-held analog goniometer. The accuracy and precision of this tool is inconsistent, and depends not only upon the type of goniometer used, but also upon the expertise of the therapist making the measurement. Additionally, traditional goniometry provides only static measurements, and continuous data cannot be gathered. This means that feedback is only offered at the beginning and end of a supervised therapy session, and no feedback is available during active exercise, or between sessions during execution of a home exercise program (HEP).

Existing strategies for continuous measurement of hand and wrist ROM that are both technically advanced and clinically sound include computer vision, wearable sensors utilizing strain or bending sensors and magnetometers, wrist motion sensing using electromyography or pressure sensors, and inertial measurement units. These advanced approaches, however, face practical challenges which limit adoption. These include occlusions and restricted workspace; the need for large training datasets; limited capabilities offering only gesture recognition rather than precise angle measurement; reliance on tethered systems with bulky benchtop electronics; the need for additional calibration equipment; and significant computational power requirements. A comparison of various technologies that can measure joint angles is presented in Supplementary Table 1 of Appendix C, highlighting key characteristics such as sensor, targeted modality, uncertainty, calibration methods, connection type, and computing and data processing platform. These characteristics may serve as criteria for evaluating the feasibility of deploying these technologies in both in-clinic and at-home rehabilitation settings, where compatibility with current rehabilitation protocols is particularly important for enhancing therapeutic outcomes.

The ability to continuously track joint angles in hand and wrist using a wireless, wearable system that conforms to the surroundings of a target joint would address such challenges in ROM biofeedback for rehabilitation. Reliable and instant reporting of this kinematic biofeedback to clinicians and patients, whether in-clinic or at-home physical therapy settings, benefits both parties and may enhance rehabilitation outcomes.

### SUMMARY OF THE INVENTION

Among the various aspects of the present disclosure is the provision of systems, devices, and related methods to monitor joint angles.

Briefly, therefore, the present disclosure is directed to a wireless wearable goniometer systems and methods for measuring joint angles.

Among the various aspects of the present disclosure is the provision of a wireless wearable goniometer system. The system comprises a wearable electrogoniometer sensor configured to detect changes in joint angles in a subject, comprising (i) a magnet attached to a first appendage segment distal to a joint, (ii) a magnetometer attached to a second appendage segment proximal to the joint, wherein the magnetometer is configured to detect changes in a magnetic field induces by movements of the magnet associated with joint rotations, and (iii) a circuit board operatively connected to the magnetometer and comprising a power source, a power management integrated circuit (PMIC), and a wireless data communication circuit, and a smart device comprises a wireless data communication circuit and a display, wherein the smart device is configured to receive signals from the wearable electrogoniometer sensor and to display measure joint angles on the display using a graphical user interface (GUI).

In some aspects, the wearable electrogoniometer sensor further comprises an attachment configuration to secure the wearable electrogoniometer sensor to the subject, selected from an adhesive, and at least one strap with a pin-and-hole design, and any combination thereof. In some aspects, the wearable electrogoniometer sensor is encased in a silicone shell. In some aspects, the silicon shell has a thickness of 0.5 mm. In some aspects, the silicone shell comprises at least two fiducial markers. In some aspects, the at least two fiducial markers comprise a pattern of silicone with white and black dye, and wherein a first fiducial marker is located above the magnet, and a second fiducial marker is located above the magnetometer. In some aspects, the power source comprises a 3.7-V lithium polymer battery. In some aspects, the circuit board further comprises a folded charging coil for wireless charging. In some aspects, the magnet comprises a stack of four rectangular neodymium magnets. In some aspects, the wearable electrogoniometer sensor is configured to measure the joint angle in a joint selected from a finger joint and a wrist joint. In some aspects, the wireless data communication circuit comprises a Bluetooth module.

Among the various aspects of the present disclosure is also the provision of a method to measure changes in a joint angle in a subject using a wearable wireless goniometer system comprising a wearable electrogoniometer sensor and a smart device. The method comprises attaching the wearable electrogoniometer sensor to a joint of the subject, calibrating the wireless goniometer system, and utilizing the wireless goniometer system to measure changes in the joint angle.

In some aspects, the wearable electrogoniometer sensor comprises (i) a magnet attached to a first appendage segment distal to a joint, (ii) a magnetometer attached to a second appendage segment proximal to the joint, wherein the magnetometer is configured to detect changes in a magnetic field induces by movements of the magnet associated with joint rotations, (iii) a circuit board operatively connected to the magnetometer and comprising a power source, a power management integrated circuit (PMIC), and a wireless data communication circuit, (iv) a silicone shell encasing the electrogoniometer sensor, and (v) at least two fiducial markers. In some aspects, the smart device comprising a wireless data communication circuit and a display, wherein the smart device is configured to receive signals from the wearable electrogoniometer sensor and to display measured joint angles on the display using a graphical user interface (GUI). In some aspects, calibrating the wireless goniometer system comprises measuring a relative angle of the magnet to the magnetometer comprising measuring a flexion/extension motion with the equation θₜ=signum ((vₚ×v_{d})×uₚ)cos⁻¹ (vₚ×v_{d}); and measuring a ulnar/radial motion with the equation θᵣ=signum ((uₚ×u_{d})×wₚ)cos⁻¹ (uₚ×u_{d}), and measuring the flexion/extension motion and the ulnar radial motion comprises the smart device tracking the fiducial markers with a camera. In some aspects, the wearable goniometer system can measure changes in the joint angle during events comprising vigorous activity and daily tasks. In some aspects, the wearable electrogoniometer sensor can be attached to the subject for a time ranging from 1 minute to 7 hours.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1 is a block diagram schematically illustrating a system in accordance with one aspect of the disclosure.

FIG. 2 is a block diagram schematically illustrating a computing device in accordance with one aspect of the disclosure.

FIG. 3 is a block diagram schematically illustrating a remote or user computing device in accordance with one aspect of the disclosure.

FIG. 4 is a block diagram schematically illustrating a server system in accordance with one aspect of the disclosure.

FIG. 5A is an exploded-view illustration of a finger goniometer (top) and a corresponding pair of images of the finger goniometer (bottom). The exploded-view illustration shows the position of the magnet, magnetometer, wireless electronics, pins, markers, and battery. The bottom images show the finger goniometer on a flat surface (left) and on a human hand (right) showing the placement of the targeted joint.

FIG. 5B is an exploded-view illustration of a wrist goniometer (top) and a corresponding pair of images of the wrist goniometer (bottom). The exploded-view illustration shows the position of the magnet, magnetometer, wireless electronics, pins, markers, and battery. The bottom images show the wrist goniometer on a flat surface (left) and on a human hand (right) showing the placement of the targeted joint.

FIG. 5C is a schematic diagram of the graphical user interface (GUI) implemented on a smart device that supports vision-based calibration and captures angles and the range of angular motions in real-time during a trial.

FIG. 5D is an image showing a measurement configuration for monitoring proximal interphalangeal joint motions of the left index finger, illustrated with an actual user.

FIG. 6A is a flow chart of the goniometer system, showing initial data collection for magnetometer response calibration and routine use with calibrated magnetometer responses. During calibration, ground truth angles (θ) are collected using the vision system. Orientation vectors (u, v, and w) of two visual markers are used to estimate joint angles using the vision system.

FIG. 6B is a pair of illustrations showing orientation vectors (u, v, and w) of two markers are used to estimate angles from flexion/extension motions. Subscripts, d, p, f, and r, denote distal, proximal, flexion/extension, and radial/ulnar, respectively. Symbols θ and φ represent joint angles and magnet angles, respectively.

FIG. 6C is a pair of illustrations showing orientation vectors (u, v, and w) of two markers used to estimate angles from radial/ulnar motions. Subscripts, d, p, f, and r, denote distal, proximal, flexion/extension, and radial/ulnar, respectively. Symbols θ and φ represent joint angles and magnet angles, respectively.

FIG. 6D is an image of the display of a smart device in use while marker tracking is active.

FIG. 6E is a graph of absolute error showing the accuracy and range of measured angles with respect to four different camera angles. Boxes represent the interquartile range (IQR) from the 25th to the 75th percentiles. Whiskers extend to 1.5 times the IQR. The midline within each box indicates the median of the dataset

FIG. 6F is a graph of detected angles showing the accuracy and range of measured angles with respect to four different camera angles. Boxes denote the range (the minimum and maximum values) for each camera angle.

FIG. 6G is a flow chart illustrating the calibration and routine use of the disclosed goniometer system, including the use of extrapolation to address the limited range of measured angles resulting from camera angles. An R² value obtained from curve fitting indicates the goodness of fit and serves as a criterion for either passing or failing the calibration step.

FIG. 7A is a set of graphs showing the device calibration with extrapolation (top), responses to the expected full range of joint motions (middle), and responses to cyclic loadings (bottom) in the electrogoniometry systems for capturing flexion/extension of the fingers. The shaded areas indicate the range of calibration data.

FIG. 7B is a set of graphs showing the device calibration with extrapolation (top), responses to the expected full range of joint motions (middle), and responses to cyclic loadings (bottom) in the electrogoniometry systems for capturing flexion/extension of the wrist. The shaded areas indicate the range of calibration data.

FIG. 7C is a set of graphs showing responses of the electrogoniometer for wrist radial/ulnar motions, including device calibration with interpolation (top), response to the expected full range of motion (middle), and response to cyclic loading (bottom). The shaded areas indicate the range of calibration data.

FIG. 8A is an image captured from high-speed videos obtained during thumb flexion/extension experiments (top), a graph showing positions of markers tracked in 3D space (middle), and a graph of joint angles estimated by 3D marker tracking alongside device measurements (bottom) during flexion/extension motions of the thumb.

FIG. 8B is an image captured from high-speed videos obtained during index finger flexion/extension experiments (top), a graph showing positions of markers tracked in 3D space (middle), and a graph of joint angles estimated by 3D marker tracking alongside device measurements (bottom) during flexion/extension motions of the index finger.

FIG. 8C is an image captured from high-speed videos obtained during wrist flexion/extension experiments (top), a graph showing positions of markers tracked in 3D space (middle), and a graph of joint angles estimated by 3D marker tracking alongside device measurements (bottom) during flexion/extension motions of the wrist.

FIG. 8D is an image captured from high-speed videos obtained during elbow flexion/extension experiments (top), a graph showing positions of markers tracked in 3D space (middle), and a graph of joint angles estimated by 3D marker tracking alongside device measurements (bottom) during flexion/extension motions of the radial/ulnar joint of the elbow.

FIG. 9A is an image of two overlaid still shots from a fluoroscopic video Tracking points are to estimate ground truth angles of the proximal interphalangeal joint (PIP) in the index finger.

FIG. 9B is a set of graphs comparing ground truth angles of the right (left) and left (right) fingers from five healthy volunteers with those measured by the disclosed goniometer device.

FIG. 9C is a Bland-Altman plot assessing the agreement between the sets of readings obtained from the fluoroscopy system and the disclosed goniometer device.

FIG. 9D is a set of graphs comparing PIP joint angles in the index, middle, and small fingers of the right (top) and left (bottom) hands of ten healthy volunteers, measured by a hand therapist using a digital goniometer, versus those measured by the device.

FIG. 9E is a Bland-Altman plot illustrating the agreement between the two measurement methods.

FIG. 9F is a set of photographs and corresponding graphs of three types of tendon glide exercises (straight fist, hook fist, reverse blocking), accompanied by time-series data of PIP joint angles in index finger collected during the exercises.

FIG. 10A is a photograph of the finger goniometer worn on the right index finger to track PIP joint motions during office work, captured over 1 hour.

FIG. 10B is a pair of graphs showing the measured range of motion in the joint angles for three healthy subjects (top), and time-series data of measured angles of Subject 1 (bottom). Boxes represent IQR from the 25th to the 75th percentiles; whiskers extend to 1.5 times the IQR; and the midline within each box indicates the median of the dataset.

FIG. 10C is a set of graphs showing long-term (7-hour) measurements of PIP joint angles in both the left and right index fingers, showing their range and time-series data. The time-series data consist of over 627,000 data points for each finger, and the range was estimated from these data. Boxes represent IQR from the 25th to the 75th percentiles; whiskers extend to 1.5 times the IQR; and the midline within each box indicates the median of the dataset.

FIG. 10D is an image of the device on the right index finger obtained during baseball throwing, executed ten times.

FIG. 10E is a set of graphs showing the measured range of motion in joint angles (top), and time-series data of Subject 1 (bottom), with red triangle markers indicating release points. Boxes represent IQR from the 25th to the 75th percentiles; whiskers extend to 1.5 times the IQR; and the midline within each box indicates the median of the dataset.

FIG. 10F is an image of the device on the right hand while playing the plano.

FIG. 10G is an image of sheet music and corresponding graph of time-series data of measured angles and a music sheet from a specific section of the performance.

FIG. 11A is a set of images of a soft, skin-interfaced wireless electrogoniometry system for monitoring motions of finger joints. The electrogoniometry systems for hand digits with their dimensions (top), optical images of the flexible printed circuit board fully populated with all components, including a magnetometer, a Bluetooth module, and power management integrated circuit (PMIC) circuitry (middle), and an image of the electrogoniometers placed on a wireless charger while being charged (bottom).

FIG. 11B is a set of images of a soft, skin-interfaced wireless electrogoniometry system for monitoring motions of wrist joints. The electrogoniometry systems for wrists with their dimensions (top), optical images of the flexible printed circuit board fully populated with all components, including a magnetometer, a Bluetooth module, and power management integrated circuit (PMIC) circuitry (middle), and an image of the electrogoniometers placed on a wireless charger while being charged (bottom).

FIG. 12A is an exploded-view view of the benchtop experimental setup to monitor flexion/extension and radial/ulnar motions of finger and wrist joints.

FIG. 12B is an image of the experimental setup, which includes a stepper motor, two manual stages for fine adjustment of joint locations, a 3D printed rod to minimize fringing magnetic fields from the motor, and a smart device mounted on a tripod.

FIG. 12C is an image of the smart device during flexion/extension motion tests with active marker tracking.

FIG. 12D is an image from a video captured during flexion/extension tests, with three markers used to estimate ground truth angles.

FIG. 12E is an image from the smart device during radial/ulnar motion tests with active marker tracking.

FIG. 12F is an image from a video captured during radial/ulnar tests, with three markers that facilitate ground truth angle estimations.

FIG. 13A is a set of images from a smart device at four different camera angles while estimating angles from quasi-static loads from four different views (view 1, view 2, view 3, view 4).

FIG. 13B is a set of plots comparing angles collected by the vision system against ground truth angles (motor angles) from four views during flexion/extension motions simulating those in finger joints.

FIG. 13C is a Bland-Altman plot assessing the agreement between the two sets of readings from FIG. 13B.

FIG. 14A is an image and corresponding graph of the vision system with dynamic flexion/extension motions simulating those in finger joints. Screen captures from a smart device at a camera angle (view 1) while estimating angles under cyclic loads (left) and a comparison of angles collected by the vision system against motor angles that are regarded as ground truth (right). This comparison includes time-series data, absolute error (|ε°|), and root mean square error (RMSE), and motor angles are resampled at 25 Hz for error estimations.

FIG. 14B is an image and corresponding graph of the vision system with dynamic flexion/extension motions simulating those in finger joints. Screen captures from a smart device at a camera angle (view 2) while estimating angles under cyclic loads (left) and a comparison of angles collected by the vision system against motor angles that are regarded as ground truth (right).

FIG. 14C is an image and corresponding graph of the vision system with dynamic flexion/extension motions simulating those in finger joints. Screen captures from a smart device at a camera angle (view 3) while estimating angles under cyclic loads (left) and a comparison of angles collected by the vision system against motor angles that are regarded as ground truth (right).

FIG. 14D is an image and corresponding graph of the vision system with dynamic flexion/extension motions simulating those in finger joints. Screen captures from a smart device at a camera angle (view 4) while estimating angles under cyclic loads (left) and a comparison of angles collected by the vision system against motor angles that are regarded as ground truth (right).

FIG. 15A is a set of images from a smart device at four different camera angles while estimating angles from quasi-static loads during flexion/extension motions simulating those in the wrist joint.

FIG. 15B is a set of plots comparing angles collected by the vision system against ground truth angles (motor angles) from four different views.

FIG. 15C is a Bland-Altman plot assessing the agreement between the two sets of readings from FIG. 15B.

FIG. 16A is an image from a smart device of dynamic flexion/extension motions simulating those in the wrist joint at different camera angles (view 1) while estimating angles under cyclic loads (left) and a graph comparing angles collected by the vision system against motor angles that are regarded as ground truth (right). This comparison includes time-series data, absolute error (|ε°|), and root mean square error (RMSE), and motor angles are resampled at 25 Hz for error estimations.

FIG. 16B is an image from a smart device of dynamic flexion/extension motions simulating those in the wrist joint at different camera angles (view 2) while estimating angles under cyclic loads (left) and a graph comparing angles collected by the vision system against motor angles that are regarded as ground truth (right).

FIG. 16C is an image from a smart device of dynamic flexion/extension motions simulating those in the wrist joint at different camera angles (view 3) while estimating angles under cyclic loads (left) and a graph comparing angles collected by the vision system against motor angles that are regarded as ground truth (right).

FIG. 16D is an image from a smart device of dynamic flexion/extension motions simulating those in the wrist joint at different camera angles (view 4) while estimating angles under cyclic loads (left) and a graph comparing angles collected by the vision system against motor angles that are regarded as ground truth (right).

FIG. 17A is a set of images of quasi-static radial/ulnar motions simulating those in the wrist joint from a smart device at four different camera angles while estimating angles from quasi-static loads.

FIG. 17B is a set of plots comparing angles collected by the vision system against ground truth angles (motor angles) from four different views.

FIG. 17C is a Bland-Altman plot assessing the agreement between the two sets of readings from FIG. 17B.

FIG. 18A is an image and corresponding graph of dynamic radial/ulnar motions simulating those in the wrist joint. The image is from a smart device at a camera angle (view 1) while estimating angles under cyclic loads (left) and a comparison of angles collected by the vision system against motor angles that are regarded as ground truth (right). This comparison includes time-series data, absolute error (|ε°|), and root mean square error (RMSE), and motor angles are resampled at 25 Hz for error estimations.

FIG. 18B is an image and corresponding graph of dynamic radial/ulnar motions simulating those in the wrist joint. The image is from a smart device at a camera angle (view 2) while estimating angles under cyclic loads (left) and a comparison of angles collected by the vision system against motor angles that are regarded as ground truth (right).

FIG. 18C is an image and corresponding graph of dynamic radial/ulnar motions simulating those in the wrist joint. The image is from a smart device at a camera angle (view 3) while estimating angles under cyclic loads (left) and a comparison of angles collected by the vision system against motor angles that are regarded as ground truth (right).

FIG. 18D is an image and corresponding graph of dynamic radial/ulnar motions simulating those in the wrist joint. The image is from a smart device at a camera angle (view 4) while estimating angles under cyclic loads (left) and a comparison of angles collected by the vision system against motor angles that are regarded as ground truth (right).

FIG. 19A is a set of images of a set-up of experiments conducted to collect reference functions for extrapolating angles measured by the computer vision system and the magnetometer during flexion/extension motions.

FIG. 19B is a set of contour maps of R2 values estimated from fitted functions relative to the locations of the magnet and magnetometer under expected ranges of motion for fingers (top) and wrist (bottom). A star mark in the right contour map denotes the selected dimension of the electrogoniometry system for wrist joints.

FIG. 20A is a set of images of the graphical user interface (GUI) of the electrogoniometry system and its operating procedure. Still shots from a screen-recorded video captured while using the electrogoniometry system illustrate: the initial state, where both computer vision (CV) and magnetometer (Mag) are turned off. A close-up view of the interface shows key functions, including the CV on-off toggle, real-time angle display, real-time plot, range of motion (ROM) display, calibration (Calib) buttons, and Calib result display;

FIG. 20B is an image of the GUI of the CV check-up mode where single-axis orientation vectors from CV are displayed on the screen. Black color in the real-time angle display and plot indicates that only CV data is used.

FIG. 20C is an image of the GUI of the calibration mode which begins when the CALIB button is pressed to correlate Mag data with CV angle data. The red color indicates the interface is in Calib mode.

FIG. 20D is an image of the GUI of routine use mode where all vision related functions are off, and Mag data is solely used. Calib result display shows the calibration result, and ROM display becomes active. The color indicator turns to blue to denote this mode.

FIG. 21A is a set of error analysis plots obtained using the data of FIG. 3A. Absolute errors (|ε°|) and root mean square error (RMSE) of the experimental results with the expected full range of joint motions (top) and cyclic loadings (bottom), simulating flexion/extension of the fingers. Motor angles (ground truth angles) are resampled at 25 Hz for error estimations.

FIG. 21B is a set of error analysis plots obtained using the data of FIG. 3B. Absolute errors (|ε°|) and root mean square error (RMSE) of the experimental results with the expected full range of joint motions (top) and cyclic loadings (bottom), simulating flexion/extension of the wrist. Motor angles (ground truth angles) are resampled at 25 Hz for error estimations.

FIG. 21C is a set of error analysis plots obtained using the data of FIG. 3C. Absolute errors (|ε°|) and root mean square error (RMSE) of the experimental results with the expected full range of joint motions (top) and cyclic loadings (bottom), simulating radial/ulnar motions of wrist. Motor angles (ground truth angles) are resampled at 25 Hz for error estimations.

FIG. 22A is a set of graphs of the electrogoniometry system for the fingers with a benchtop setup. Device calibration with extrapolation (top), responses to the expected full range of joint motions (middle), and responses to cyclic loadings (bottom) in the electrogoniometry systems for capturing flexion/extension of the fingers. The middle and bottom graphs include a comparison of angles collected by the vision system against motor angles that are regarded as ground truth. This comparison includes time-series data, absolute error (|ε°|), and root mean square error (RMSE), and motor angles are resampled at 25 Hz for error estimations.

FIG. 22B is a set of graphs of the electrogoniometry system for the fingers with a benchtop setup. Device calibration with extrapolation (top), responses to the expected full range of joint motions (middle), and responses to cyclic loadings (bottom) in the electrogoniometry systems for capturing flexion/extension of the fingers.

FIG. 23A is a set of graphs of the electrogoniometry system for the wrist with a benchtop setup. Device calibration with extrapolation (top), responses to the expected full range of joint motions (middle), and responses to cyclic loadings (bottom) in the electrogoniometry systems for capturing flexion/extension of the wrist. The middle and bottom graphs include a comparison of angles collected by the vision system against motor angles that are regarded as ground truth. This comparison includes time-series data, absolute error (|ε°|), and root mean square error (RMSE), and motor angles are resampled at 25 Hz for error estimations.

FIG. 23B is a set of graphs of the electrogoniometry system for the wrist with a benchtop setup. Device calibration with extrapolation (top), responses to the expected full range of joint motions (middle), and responses to cyclic loadings (bottom) in the electrogoniometry systems for capturing flexion/extension of the wrist.

FIG. 24A is a schematic of a bench top setup applies flexion/extension motions (−18° to) 110° to the device with three different sets of magnet and magnetometer locations: Set1 (x1=18.5 mm and x2=6.5 mm; blue), Set2 (x1=23.5 mm and x2=11.5 mm, orange), and Set3 (x1=28.5 mm and x2=16.5 mm, yellow).

FIG. 24B is a graph of device responses to the flexion/extension motions show an overall sensitivity of 2.3° joint angle/1o magnet orientation (2.1 for Set1, 2.3 for Set2, and 2.6 for Set3). A blue dashed rectangle indicates a close-up view in FIG. 24C.

FIG. 24C is a graph of device responses to the flexion/extension motions of 2.3° joint angle/1° magnet orientation (2.1 for Set1, 2.3 for Set2, and 2.6 for Set3) with restricted motion range (0° to) 40°.

FIG. 24D is a graph of magnetometer readings along x-axis for set 1 obtained using the data from FIG. 24B.

FIG. 24E is a graph of magnetometer readings along x-axis for set 2 obtained using the data from FIG. 24B.

FIG. 24F is a graph of magnetometer readings along x-axis for set 3 obtained using the data from FIG. 24B.

FIG. 24G is a graph of magnetometer readings along z-axis for set 1 obtained using the data from FIG. 24B.

FIG. 24H is a graph of magnetometer readings along z-axis for set 2 obtained using the data from FIG. 24B.

FIG. 24I is a graph of magnetometer readings along z-axis for set 3 obtained using the data from FIG. 24B.

FIG. 25A is a schematic of the electrogoniometry system for the fingers in a benchtop setup. A bench top setup applies the flexion/extension motions (−18° to) 110° to the device with four different magnet locations: From Loc1 to 4, the y position (deviation from the center) increases with increments of 2.5 mm. Loc1 is at the center.

FIG. 25B is a plot of device responses to the flexion/extension motions. A blue dashed rectangle illustrates a close-up view in FIG. 25C.

FIG. 25C is a plot of device responses to the flexion/extension motions at a range of −5° to 5°.

FIG. 26A is set of images of electrogoniometry systems at different ages: Old1 (1.5 years; left), Old2 (1 year; center), and New (right).

FIG. 26B is a plot of responses of each device across three trials of flexion motions, ranging from 10° to 30° in increments of 1°, show a standard deviation of less than 0.3°. The standard deviation of the mean across devices is less than 0.4°.

FIG. 26C is a plot of a close-up view of responses from FIG. 26B with the responses being within the 13° to 17° range.

FIG. 26D is a plot of time-series responses to step loads (increments of 10) of the devices obtained using the data from FIG. 26A.

FIG. 26E is a plot of the root mean square error (RMSE) at each step of the devices obtained using the data from FIG. 26A.

FIG. 27A is a schematic of electrogoniometry system for the wrist in a benchtop setup. A bench top setup applies flexion/extension motions (−70° to) 70° to the device with three different sets of magnet and magnetometer locations: Set1 (x1=24 mm and x2=9 mm; blue), Set2 (x1=29 mm and x2=14 mm, orange), and Set3 (x1=34 mm and x2=19 mm, yellow).

FIG. 27B is a plot of device responses to the flexion/extension motions show an overall sensitivity of 3.8° joint angle/1φ magnet orientation (3.2 for Set1, 3.6 for Set2, and 4.7 for Set3). A blue dashed rectangle indicates the data shown in the close-up view of FIG. 27C.

FIG. 27C is a plot of device responses to the flexion/extension motions showing a close-up view of data within the blue dashed rectangle in FIG. 27B, showing a restricted motion range of −20° to 20°.

FIG. 27D is a plot of device responses to flexion/extension motions ranging from −9° to 9° in increments of 1° show a standard deviation of less than 0.7° from three trials.

FIG. 27E is a plot of device responses to the flexion/extension motions of a close-up view of FIG. 27D, showing the responses with the −2° to 2° range.

FIG. 27F is a plot of RMSE at each loading step (ground truth) of the device in FIG. 27A.

FIG. 28A is a schematic of the electrogoniometry system for the wrist in a benchtop setup. A bench top setup applies radial/ulnar motions (−50° to) 50° to the device with three different sets of magnet and magnetometer locations: Set1 (x1=25 mm and x2=16.5 mm; blue), Set2 (x1=27.5 mm and x2=14 mm, orange), and Set3 (x1=30 mm and x2=16.5 mm, yellow).

FIG. 28B is a plot of device responses to the radial/ulnar motions show an overall sensitivity of −4.20 joint angle/1φ magnet orientation (−4.1 for Set1, −4.0 for Set2, and −4.4 for Set3). A blue dashed rectangle indicates a close-up view in FIG. 28C.

FIG. 28C is a plot of device responses to the radial/ulnar motions showing a close-up view of data enclosed by the blue dashed rectangle FIG. 28B, showing a restricted motion range of −20° to 20°.

FIG. 28D is a plot of device responses to radial/ulnar motions ranging from −9° to 9° in increments of 1° show a standard deviation of less than 0.7° from three trials.

FIG. 28E is a plot of device responses to the radial/ulnar motions of a close-up view of FIG. 27D, showing the responses with the −2° to 2° range.

FIG. 28F is a plot of RMSE at each loading step (ground truth) of the device in FIG. 28A.

FIG. 29A is a set of plots showing device responses to angular loads spanning from −14° to 51° at three different speeds (ω1≈55%; ω2≈75°/s; ω3≈125°/s) continuously applied for 45 minutes (top). Device responses to five cycles of the load at each speed are shown at the bottom (left, 704-716s; middle, 1630-1637 s; right, 2215-2218s).

FIG. 29B is a set of images of the experimental setup to investigate drift in the device under temperature variations ranging from 23.5 °C to 40 °C. A close-up view shows the device secured on an aluminum plate inside an oven (AccuTemp, Across International), and photographs of the oven's digital screen illustrate the temperatures at the start and end of the experiment.

FIG. 29C is a plot of device responses to temperature variations, showing a change in angle of less than 1.7°.

FIG. 30 is a plot showing device responses to dynamic loads ranging from −14° to 51°, applied for ten cycles at each of three different angular speeds (ω1≈55°/s, top; ω2≈ 75°/s, middle; ω3=125°/s, bottom), showing negligible hysteresis across the applied dynamic loads.

FIG. 31A is a schematic illustration of the elements of a 3D marker tracking system with four synchronized high-speed cameras (left) and an image of an experimental setup for implementing 3D marker tracking (right).

FIG. 31B is an algorithmic flow chart of 3D marker tracking showing the 3D reconstruction and markerless tracking.

FIG. 31C is a plot comparing angles measured by 3D marker tracking against five ground truth angles. Inset pictures show 3D-printed parts with ground truth angles spanning from 15° to 75° in 15° increments.

FIG. 31D is a plot of RMSE of measured angles from three trials at each ground truth angle.

FIG. 32A is a plot of error analysis based on the data of FIG. 4A. Absolute errors (|ε°|) and root mean square error (RMSE) of the experimental results with 3D marker tracking for flexion/extension of thumb. Angles from 3D marker tracking are resampled at 25 Hz for error estimations.

FIG. 32B is a plot of error analysis based on the data of FIG. 4B. Absolute errors (|ε°|) and root mean square error (RMSE) of the experimental results with 3D marker tracking for flexion/extension of the index finger. Angles from 3D marker tracking are resampled at 25 Hz for error estimations.

FIG. 32C is a plot of error analysis based on the data of FIG. 4C. Absolute errors (|ε°|) and root mean square error (RMSE) of the experimental results with 3D marker tracking for flexion/extension of the wrist. Angles from 3D marker tracking are resampled at 25 Hz for error estimations.

FIG. 32D is a plot of error analysis based on the data of FIG. 4D. Absolute errors (|ε°|) and root mean square error (RMSE) of the experimental results with 3D marker tracking for flexion/extension of radial/ulnar motions of wrist. Angles from 3D marker tracking are resampled at 25 Hz for error estimations.

FIG. 33A is an image and set of graphs of the performance of the electrogoniometry system characterized through 3D marker tracking. Still shots from high-speed videos captured during user experiments (left), positions of markers tracked in 3D space (center), and joint angles estimated by 3D marker tracking alongside device measurements (right) during flexion/extension motions of the middle finger. A comparison in the right figure includes absolute error (|ε°|) and root mean square error (RMSE), and angles from 3D marker tracking are resampled at 25 Hz for error estimations.

FIG. 33B is an image and set of graphs of the performance of the electrogoniometry system characterized through 3D marker tracking. Still shots from high-speed videos captured during user experiments (left), positions of markers tracked in 3D space (center), and joint angles estimated by 3D marker tracking alongside device measurements (right) during flexion/extension motions of the ring finger.

FIG. 33C is an image and set of graphs of the performance of the electrogoniometry systems characterized through 3D marker tracking. Still shots from high-speed videos captured during user experiments (left), positions of markers tracked in 3D space (center), and joint angles estimated by 3D marker tracking alongside device measurements (right) during flexion/extension motions of the little finger.

FIG. 34A is a set of images of the electrogoniometry system worn on the index finger. A finger splint restricts the index finger's range of motion.

FIG. 34B is a plot of time-series responses of the device alongside the ground truth of the device relative to the ground truth at each step. Red triangles indicate each step.

FIG. 34C is a plot of time-series responses of the device alongside the root mean square error (RMSE) of the device relative to the ground truth at each step. Red triangles in FIG. 34B indicate each step.

FIG. 35A is an image (left) and magnified image (right) of the experimental setup used to apply artificial tremors while using the electrogoniometry system. A vibration generator oscillates a stimulus probe attached to it at frequencies set by an arbitrary waveform generator that supplies a 5V peak-to-peak square wave to the shaker. A close-up view shows a finger splint that prevents the index finger from undergoing angular motions due to artificial tremors.

FIG. 35B is a plot of device responses to artificial tremors at frequencies ranging from 0 Hz to 10 Hz in increments of 2 Hz.

FIG. 36A is an image of wrist motion (top; flex/extend) and corresponding plots of response of the electrogoniometry system for the wrist during various wrist motions. One degree of freedom (DOF) motion is assumed. Time-series responses of the electrogoniometry system alongside the ground truth for each DOF (middle) and RMSE at each location where wrist motions pause (right). Blue and red triangles in the middle figures denote the pause locations for flexion/extension and radial/ulnar motions, respectively. The same color scheme is applied in the RMSE plots.

FIG. 36B is an image of wrist motion (top; radial/ulnar) and corresponding plots of response of the electrogoniometry system for the wrist during various wrist motions. One DOF motion is assumed. Time-series responses of the electrogoniometry system alongside the ground truth for each DOF (middle) and RMSE at each location where wrist motions pause (right). Blue and red triangles in the middle figures denote the pause locations for flexion/extension and radial/ulnar motions, respectively. The same color scheme is applied in the RMSE plots.

FIG. 36C is an image of wrist motion (top; flex/extend+radial/ulnar) and corresponding plots of response of the electrogoniometry system for the wrist during various wrist motions. Two DOF motion is assumed. Time-series responses of the electrogoniometry system alongside the ground truth for each DOF (middle) and RMSE at each location where wrist motions pause (right). Blue and red triangles in the middle figures denote the pause locations for flexion/extension and radial/ulnar motions, respectively. The same color scheme is applied in the RMSE plots.

FIG. 37A is an illustration and corresponding image of human trials using a mini C-arm fluoroscopy, with a hand surgeon supervising the proximal interphalangeal (PIP) joint motions in the index fingers of five healthy volunteers.

FIG. 37B is an illustration and corresponding image of human trials conducted by a hand therapist; the accompanying photograph illustrates how the hand therapist measures the angles of the PIP joint in the left index finger of a healthy volunteer using a digital goniometer.

FIG. 38 is a set of plots showing the experimental results from human trials involving ten healthy volunteers with a hand therapist. Bland-Altman plots to appraise the difference between the proximal interphalangeal joint angles measured by the electrogoniometer and those measured by a hand therapist using digital goniometer, for the index (top), middle (middle), and small (bottom) fingers of both hands.

FIG. 39A is a plot of time-series data of proximal interphalangeal joint angles in the index fingers (right index finger, top; left index finger, bottom) of Subject 2 during one hour of office work.

FIG. 39B is a plot of time-series data of proximal interphalangeal joint angles in the index fingers (right index finger, top; left index finger, bottom) of Subject 3 during one hour of office work.

FIG. 40A is a plot of time-series data of proximal interphalangeal joint angles in the right index finger of Subject 2 during baseball throwing, with red triangle markers indicating release points.

FIG. 40B is a plot of time-series data of proximal interphalangeal joint angles in the right index finger of Subject 3 during baseball throwing, with red triangle markers indicating release points.

FIG. 41 is a piece of sheet music (top) and corresponding graph (bottom) of three trials of time-series data showing measured angles of the PIP joint in index fingers during the performance of the music.

FIG. 42A is a set of plots showing the experimental results from human trials involving ten healthy volunteers using the electrogoniometer. Time-series data of proximal interphalangeal joint angles (top) and angular velocities (upper middle) of a single volunteer, along with the angular velocities (lower middle) and their maximum and minimum values (bottom) of all volunteers, for the index finger. Boxes in the bottom panels represent the interquartile range (IQR) from the 25th to the 75th percentiles; whiskers extend to 1.5 times the IQR; and the midline within each box indicates the median of the dataset.

FIG. 42B is a set of plots showing the experimental results from human trials involving ten healthy volunteers using the electrogoniometer. Time-series data of proximal interphalangeal joint angles (top) and angular velocities (upper middle) of a single volunteer, along with the angular velocities (lower middle) and their maximum and minimum values (bottom) of all volunteers, for the middle finger. Boxes in the bottom panels represent the IQR from the 25th to the 75th percentiles; whiskers extend to 1.5 times the IQR; and the midline within each box indicates the median of the dataset.

FIG. 42C is a set of plots showing the experimental results from human trials involving ten healthy volunteers using the electrogoniometer. Time-series data of proximal interphalangeal joint angles (top) and angular velocities (upper middle) of a single volunteer, along with the angular velocities (lower middle) and their maximum and minimum values (bottom) of all volunteers, for the small finger. Boxes in the bottom panels represent the IQR from the 25th to the 75th percentiles; whiskers extend to 1.5 times the IQR; and the midline within each box indicates the median of the dataset.

### DETAILED DESCRIPTION OF THE INVENTION

Disclosed herein are wireless, wearable goniometry systems and devices that provide continuous, real-time measurements of joint angles in hand and wrist, displayed through a graphical user interface (GUI) on a computing device including, but not limited to, a smart device in various aspects. The unique design of the disclosed device, equipped with optimized data flow, facilitates seamless integration of computer vision with a magnetometer, effectively overcoming obstacles commonly encountered in existing wearable goniometers, such as the need for motion capture systems for per-use calibration, being tethered to benchtop electronics, and requiring significant computational power. Importantly, the disclosed calibration methodology formulates the relationship between local orientation of a magnet and target joint angles in a simple polynomial function, thereby avoiding iterative solvers, machine learning, or kinematic modeling typically required in magnet-based tracking of hand motions.

Additionally, the disclosed methodology customizes the calibration function for each use, enabling the electrogoniometry system to be highly adaptable across trials and users. Once calibrated, the system relies solely on magnetometer data, thus addressing typical limitation of vision-based system, such as occlusions and restricted workspace. Results from physical simulations and comparisons with two different ground truth methods—3D marker tracking under a multi-camera setup and 2D markerless tracking from fluoroscopy videos—demonstrate the favorable characteristics of the proposed systems, including high accuracy and precision (error <5°) that provide for clinically acceptable performance. As described in the examples herein, human trials in ten healthy volunteers confirm that these systems perform well in the setting of clinically relevant hand therapy exercises. In some aspects, the disclosed wearable device is operated using only a standard smart-device, and requires no additional equipment for calibration. As further demonstrated in the examples herein, experiments with vigorous activities and daily tasks over long-term use of up to 7 hours highlight the potential of the systems for applications beyond the primary clinical use.

In various aspects, soft, wireless electrogoniometry systems capable of continuous monitoring of the joints in the fingers or wrists are disclosed. The discrete design, soft mechanical properties, and wireless operational capabilities facilitate ergonomic wear without mechanically loading or constraining natural motions of targeted joints, a common issue with alternatives that use strain gauges. In various aspects, the disclosed system is configured to monitor joint angles of joints including, but not limited to, proximal interphalangeal (PIP) joints, and wrist joints. The disclosed system includes a sensor device for monitoring joint angles in the form of a wearable finger goniometer (FG).

In some aspects, the wearable finger goniometer (FG) includes at least one strap band with a pin-and-hole design, allowing users to wear the device without the need for adhesives or additional straps, rendering the device suitable for use by post-surgical patients. In one aspect, a wrist goniometer (WG) includes a least one hole and pin fastener that provides for separate segments to be connected or disconnected as needed. The hole and pin also serve as a fiducial to facilitate initial positioning of the device on the wrist while the two segments are connected. Similar to the fulcrum of an analog goniometer used in standard clinical practice, this fiducial, placed over the capitate on the dorsal aspect of the wrist joint, ensures proper alignment of the magnet and magnetometer. The magnet is positioned on the dorsal aspect of the hand along the third metacarpal, while the magnetometer is placed on the distal forearm, aligning with the dorsal midline of the forearm.

In various additional aspects, the two segments of the FG and WG include additional features encapsulated in soft silicone enclosures. In some aspects, a thin, flexible printed circuit board (fPCB) and a 3.7-V lithium polymer battery (45 mAh; dimensions: 15 mm length, 12 mm width, and 4 mm thickness) encased in medical-grade silicone shells (thickness: 0.5 mm; Silbione RTV 4420, Elkem), forms one segment. In other aspects, a stack of four rectangular neodymium magnets (grade N50; dimensions: 6 mm length, 3 mm width, and 2 mm thickness) forms another segment. These magnets form a cuboid structure with a height of 3 mm, to produce a strong magnetic field in a low-profile design. The FG and WG sensor devices may include different architectures defining distinct electrogoniometry systems, each tailored to the finger and wrist joints, respectively.

In various additional aspects, the soft silicon enclosures not only to isolate the electronics from the environment but also include embedded fiducial markers to facilitate calibration of joint angle measurements using computer vision methods (i.e., ArUco marker). In some aspects, patterns of silicone with white and black dye define ArUco markers with high contrast, ensuring the accuracy and reliability of joint angle measurements using computer vision for purposes of calibration with a smart device including, but not limited to, a smart phone, smart tablet, or other suitable smart device.

In various aspects, the flexible printed circuit boards (fPCBs) support and interconnect a collection of electronic components, including a magnetometer (MLX90393, Melexis), a power management integrated circuit (PMIC) with a wireless charging coil, and a Bluetooth Low Energy (BLE) system on a chip (SoC; ISP-1807, Insight SIP). This system provides digital measurements of magnetic field strength along three axes. Along with the PMIC, a foldable charging coil facilitates wireless charging using near-field communication standards. The magnetometer protrudes from the main fPCB body to align with an ArUco marker. For the FG sensor, the magnetometer connects to the main body by a serpentine wire that bypasses the non-targeting joint. This design reduces the sensitivity of the goniometer to motions of non-targeted joints and allows the main body to be placed on the back of the hand.

In various aspects, the smart device of the system is configured to display quantitative information on joint angles via a real-time GUI. In some aspects, the GUI is configured to display the quantitative information at 25-ms intervals, an interval sufficiently fast to capture natural kinematics of the finger and wrist. In some aspects, data communication between the smart device and the BLE SoC utilizes standard BLE protocols, ensuring smooth transmission of magnetometer data along with their timestamps. Along with real-time data, the GUI estimates and continuously updates minimum and maximum angles during use, to show the range of motion. Moreover, the GUI, integrated with the Open Source Computer Vision Library (OpenCV), accurately measures joint angles by tracking ArUco markers with the camera of the smart device. This information serves to calibrate the magnetometer responses to joint motions. The features of these systems renders them suitable for use in both clinical and home settings.

In various aspects, the disclosed wearable goniometer and the computer vision system of the smart device together may be used to implement procedures for calibration and subsequent routine use in monitoring joint movements. In some aspects, the calibration procedure utilizes angles determined from the computer vision system and the magnetometer. Once calibrated, the routine use of the system relies only on magnetometer data to obtain changes in joint angle as described herein. The magnetometer transmits changes in magnetic field strengths along each of three axes (Bᵢ, where i=x, y, and z) of a 3D Cartesian coordinate system to the BLE SoC at 30 Hz using the Inter-Integrated Circuit (I²C) protocol. These Bᵢ values facilitate the calculation of relative angles of the magnet cuboid to the magnetometer by using two Bᵢ on a targeting plane. For example, flexion/extension (flex/ext) motions occur in the yz-plane, and the angle resulting from these spatial motions can be defined as φ_{f}=a tan (B_{z}/B_{y}). Similarly, Bx and B_{y} on the xy-plane determine the angle from radial/ulnar (rad/uln) motions as φᵣ=a tan (Bₓ/B_{y}).

Even though φ changes along with changes in joint angles (θ), φ cannot directly represent θ because the center of rotation for φ is at the magnetometer, whereas the joint itself serves as the center of rotation for θ. The initial calibration step effectively addresses this limitation by establishing a relationship between e and φ as a function of φ. The computer vision system with ArUco marker provides estimates of θ during the calibration step. Utilizing the embedded camera with OpenCV library and SceneKit in a custom app, the smart device tracks the positions and orientations of the two ArUco markers at 60 frames per second (fps).

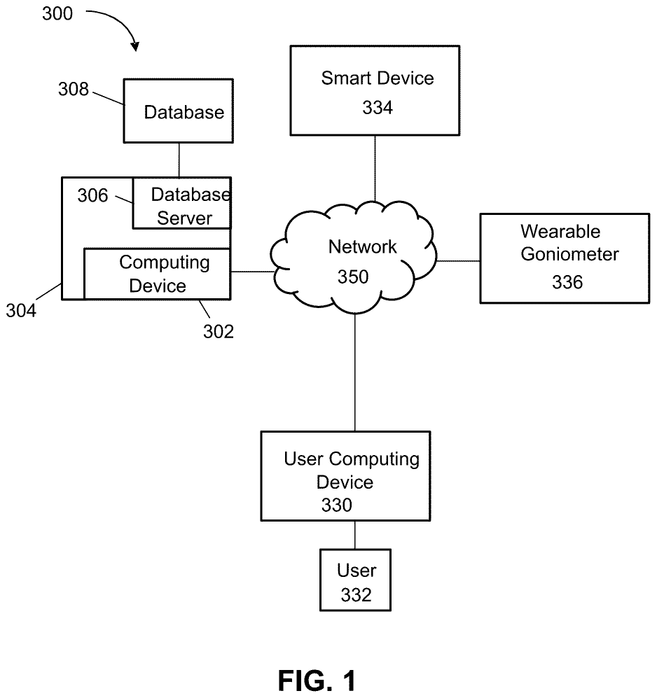

In various aspects, at least a portion of the methods disclosed herein may be implemented using various computing systems and devices as described below. FIG. 1 depicts a simplified block diagram of a computer system 300 for implementing the goniometer system and methods described herein. As illustrated in FIG. 1, the computer system 300 may be configured to implement at least a portion of the tasks associated with the disclosed joint motion monitoring system, including, but not limited to: receiving magnetometer and computer vision data from the wearable goniometer 336, transforming the magnetometer and computer vision data into a joint angle, and displaying the joint angle to a user. In an exemplary embodiment, the joint angle data can be continuously collected over multiple hours and during various levels of physical activity. The computer system 300 may include a computing device 302. In one aspect, the computing device 302 is part of a server system 304, which also includes a database server 306. The computing device 302 is in communication with database 308 through the database server 306. The computing device 302, user-computing device 330, smart device 334, and wearable goniometer 336 are communicably coupled to one another through a network 350. The network 350 may be any network that allows local area or wide area communication between the devices. For example, the network 350 may allow communicative coupling to the Internet through at least one of many interfaces including, but not limited to, at least one of a network, such as the Internet, a local area network (LAN), a wide area network (WAN), an integrated services digital network (ISDN), a dial-up-connection, a digital subscriber line (DSL), a cellular phone connection, and a cable modem. The user-computing device 330 may be any device capable of accessing the Internet including, but not limited to, a desktop computer, a laptop computer, a personal digital assistant (PDA), a cellular phone, a smartphone, a tablet, a phablet, wearable electronics, smartwatch, or other web-based connectable equipment or mobile devices.

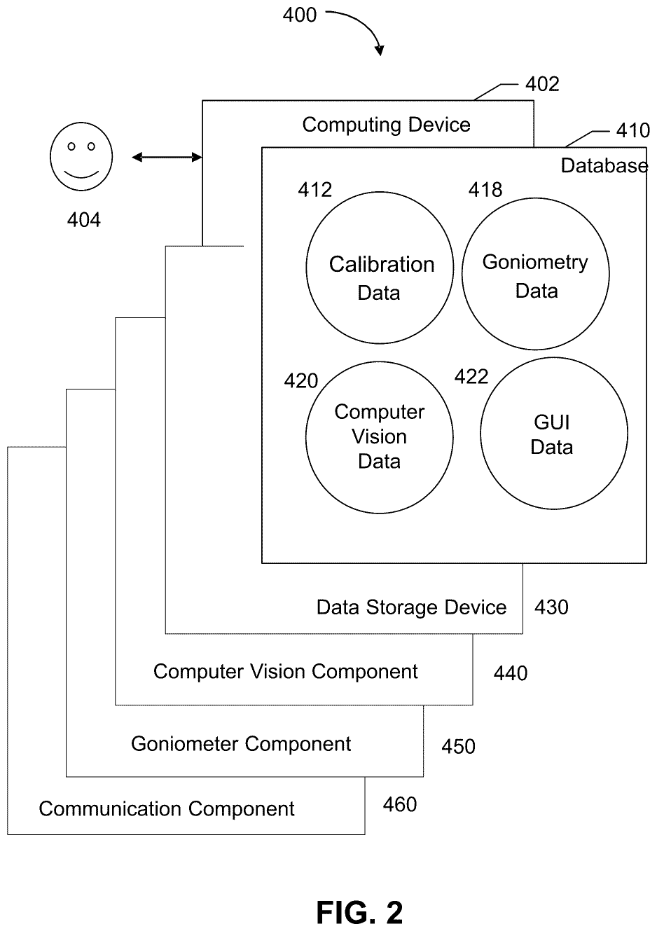

In other aspects, the computing device 302 is configured to perform a plurality of tasks associated with measuring joint angle and displaying it on the smart device GUI, including the calibration and routine use functions of the wearable goniometer. FIG. 2 depicts a system 400 with a computing device 402, which includes database 410 along with other related computing components. In some aspects, computing device 402 is similar to computing device 302 (shown in FIG. 1). A user 404 may access components of computing device 402. In some aspects, database 410 is similar to database 308 (shown in FIG. 1).

In one aspect, database 410 includes goniometry data 418, calibration data 412, computer vision data 420, and GUI data. Non-limiting examples of goniometry data 418 include any data characterizing the raw measurements obtained using the wearable goniometer and/or joint angles derived from the raw goniometer measurements. Non-limiting examples of suitable calibration data 412 include any values of parameters defining a calibration used to convert raw goniometer measurements to joint angles. Non-limiting examples of suitable computer vision data 420 may include parameters defining processes used to obtain images of fiducial markers using the camera of the smart phone and convert the images into computer vision-based joint angles. Non-limiting examples of suitable GUI data include various parameter values, symbols, shapes, and/or images used to define and display the joint angle values using the smart device. Computing device 402 also includes a number of components that perform specific tasks. In the exemplary aspect, computing device 402 includes a data storage device 430, a computer vision component 440, a goniometer component 450, and a communication component 460. Data storage device 430 is configured to store data received or generated by computing device 402, such as any of the data stored in database 410 or any outputs of processes implemented by any component of computing device 402. The computer vision component 440 is configured to obtain and analyze images using the disclosed smart device and to calculate joint angles obtained by analysis of the images. The goniometer component 450 is configured to receive and analyze signals from the goniometer device to produce joint angles during calibration and regular use of the goniometer device.

Communication component 460 is configured to enable communications between computing device 402 and other devices (e.g. user computing device 330, smart device 334, and wearable goniometer 336, shown in FIG. 1) over a network, such as network 350 (shown in FIG. 1), or a plurality of network connections using predefined network protocols such as TCP/IP (Transmission Control Protocol/Internet Protocol).

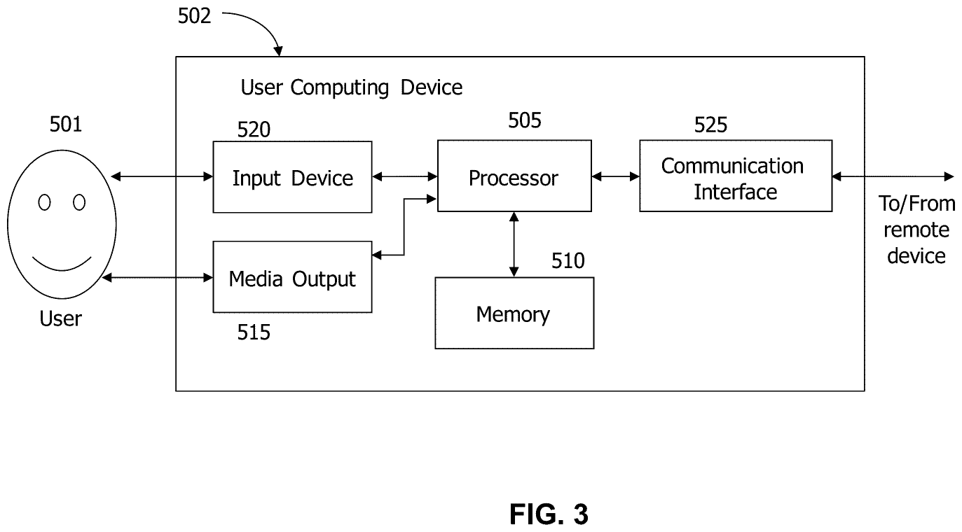

FIG. 3 depicts a configuration of a remote or user-computing device 502, such as user computing device 330 (shown in FIG. 1). Computing device 502 may include a processor 505 for executing instructions. In some aspects, executable instructions may be stored in a memory area 510. Processor 505 may include one or more processing units (e.g., in a multi-core configuration). Memory area 510 may be any device allowing information such as executable instructions and/or other data to be stored and retrieved. Memory area 510 may include one or more computer-readable media.

Computing device 502 may also include at least one media output component 515 for presenting information to a user 501. Media output component 515 may be any component capable of conveying information to user 501. In some aspects, media output component 515 may include an output adapter, such as a video adapter and/or an audio adapter. An output adapter may be operatively coupled to processor 505 and operatively coupleable to an output device such as a display device (e.g., a liquid crystal display (LCD), organic light emitting diode (OLED) display, cathode ray tube (CRT), or “electronic ink” display) or an audio output device (e.g., a speaker or headphones). In some aspects, media output component 515 may be configured to present an interactive user interface (e.g., a web browser or client application) to user 501.

In some aspects, computing device 502 may include an input device 520 for receiving input from user 501. Input device 520 may include, for example, a keyboard, a pointing device, a mouse, a stylus, a touch-sensitive panel (e.g., a touchpad or a touch screen), a camera, a gyroscope, an accelerometer, a position detector, and/or an audio input device. A single component such as a touch screen may function as both an output device of media output component 515 and input device 520.

Computing device 502 may also include a communication interface 525, which may be communicatively coupleable to a remote device. Communication interface 525 may include, for example, a wired or wireless network adapter or a wireless data transceiver for use with a mobile phone network (e.g., Global System for Mobile communications (GSM), 3G, 4G, or Bluetooth) or other mobile data network (e.g., Worldwide Interoperability for Microwave Access (WIMAX)).

Stored in memory area 510 are, for example, computer-readable instructions for providing a user interface to user 501 via media output component 515 and, optionally, receiving and processing input from input device 520. A user interface may include, among other possibilities, a web browser and client application. Web browsers enable users 501 to display and interact with media and other information typically embedded on a web page or a website from a web server. A client application allows users 501 to interact with a server application associated with, for example, a vendor or business.

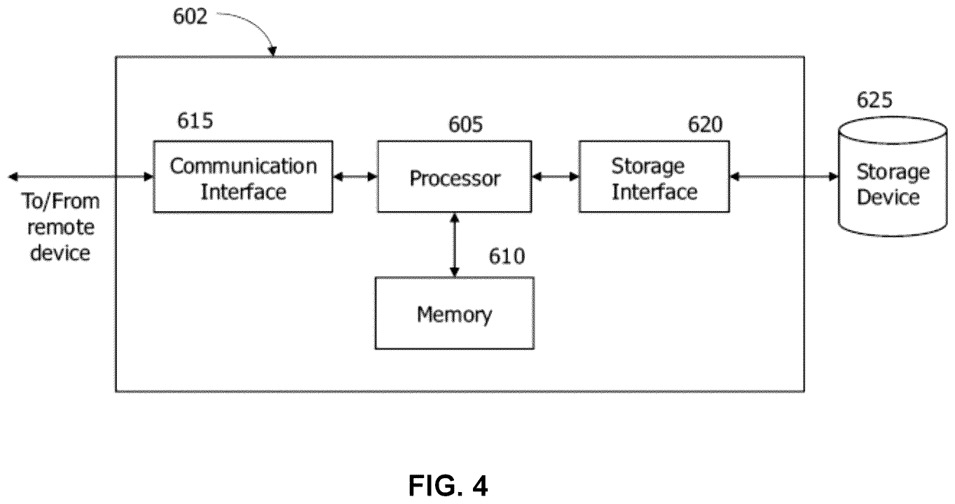

FIG. 4 illustrates an example configuration of a server system 602. Server system 602 may include, but is not limited to, database server 306 and computing device 302 (both shown in FIG. 1). In some aspects, server system 602 is similar to server system 304 (shown in FIG. 1). Server system 602 may include a processor 605 for executing instructions. Instructions may be stored in a memory area 610, for example. Processor 605 may include one or more processing units (e.g., in a multi-core configuration).

Processor 605 may be operatively coupled to a communication interface 615 such that server system 602 may be capable of communicating with a remote device such as user computing device 330 (shown in FIG. 1) or another server system 602. For example, communication interface 615 may receive requests from user computing device 330 via a network 350 (shown in FIG. 1).

Processor 605 may also be operatively coupled to a storage device 625. Storage device 625 may be any computer-operated hardware suitable for storing and/or retrieving data. In some aspects, storage device 625 may be integrated into server system 602. For example, server system 602 may include one or more hard disk drives as storage device 625. In other aspects, storage device 625 may be external to server system 602 and may be accessed by a plurality of server systems 602. For example, storage device 625 may include multiple storage units such as hard disks or solid-state disks in a redundant array of inexpensive disks (RAID) configuration. Storage device 625 may include a storage area network (SAN) and/or a network attached storage (NAS) system.

In some aspects, processor 605 may be operatively coupled to storage device 625 via a storage interface 620. Storage interface 620 may be any component capable of providing processor 605 with access to storage device 625. Storage interface 620 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 605 with access to storage device 625.

Memory areas 510 (shown in FIG. 3) and 610 may include, but are not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are examples only and are thus not limiting as to the types of memory usable for storage of a computer program.

The computer systems and computer-implemented methods discussed herein may include additional, less, or alternate actions and/or functionalities, including those discussed elsewhere herein. The computer systems may include or be implemented via computer-executable instructions stored on non-transitory computer-readable media. The methods may be implemented via one or more local or remote processors, transceivers, servers, and/or sensors (such as processors, transceivers, servers, and/or sensors mounted on vehicle or mobile devices, or associated with smart infrastructure or remote servers), and/or via computer-executable instructions stored on non-transitory computer-readable media or medium.

In some aspects, a computing device is configured to implement machine learning, such that the computing device “learns” to analyze, organize, and/or process data without being explicitly programmed. Machine learning may be implemented through machine learning (ML) methods and algorithms. In one aspect, a machine learning (ML) module is configured to implement ML methods and algorithms. In some aspects, ML methods and algorithms are applied to data inputs and generate machine learning (ML) outputs. Data inputs may further include sequencing data, sensor data, image data, video data, telematics data, authentication data, authorization data, security data, mobile device data, geolocation information, transaction data, personal identification data, financial data, usage data, weather pattern data, “big data” sets, and/or user preference data. In some aspects, data inputs may include certain ML outputs.

In some aspects, at least one of a plurality of ML methods and algorithms may be applied, which may include but are not limited to linear or logistic regression, instance-based algorithms, regularization algorithms, decision trees, Bayesian networks, cluster analysis, association rule learning, artificial neural networks, deep learning, dimensionality reduction, and support vector machines. In various aspects, the implemented ML methods and algorithms are directed toward at least one of a plurality of categorizations of machine learning, such as supervised learning, unsupervised learning, and reinforcement learning.

In one aspect, ML methods and algorithms are directed toward supervised learning, which involves identifying patterns in existing data to make predictions about subsequently received data. Specifically, ML methods and algorithms directed toward supervised learning are “trained” through training data, which includes example inputs and associated example outputs. Based on the training data, the ML methods and algorithms may generate a predictive function that maps outputs to inputs and utilize the predictive function to generate ML outputs based on data inputs. The example inputs and example outputs of the training data may include any of the data inputs or ML outputs described above.

In another aspect, ML methods and algorithms are directed toward unsupervised learning, which involves finding meaningful relationships in unorganized data. Unlike supervised learning, unsupervised learning does not involve user-initiated training based on example inputs with associated outputs. Rather, in unsupervised learning, unlabeled data, which may be any combination of data inputs and/or ML outputs as described above, is organized according to an algorithm-determined relationship.

In yet another aspect, ML methods and algorithms are directed toward reinforcement learning, which involves optimizing outputs based on feedback from a reward signal. Specifically, ML methods and algorithms directed toward reinforcement learning may receive a user-defined reward signal definition, receive a data input, utilize a decision-making model to generate an ML output based on the data input, receive a reward signal based on the reward signal definition and the ML output, and alter the decision-making model so as to receive a stronger reward signal for subsequently generated ML outputs. The reward signal definition may be based on any of the data inputs or ML outputs described above. In one aspect, an ML module implements reinforcement learning in a user recommendation application. The ML module may utilize a decision-making model to generate a ranked list of options based on user information received from the user and may further receive selection data based on a user selection of one of the ranked options. A reward signal may be generated based on comparing the selection data to the ranking of the selected option. The ML module may update the decision-making model such that subsequently generated rankings more accurately predict a user selection.

The methods and algorithms of the invention may be enclosed in a controller or processor. Furthermore, methods and algorithms of the present invention can be embodied as a computer-implemented method or methods for performing such computer-implemented method or methods, and can also be embodied in the form of a tangible or non-transitory computer-readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer programs include, for example, floppy disks and diskettes, compact disk (CD)-ROMs (whether or not writeable), DVD digital disks, RAM and ROM memories, computer hard drives and back-up drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general-purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1—Soft, Skin-Interfaced Wireless Electrogoniometry System for Continuous Monitoring of Finger and Wrist Joints

Continuous kinematic biofeedback during exercise interventions can lead to improved therapeutic outcomes in hand and wrist rehabilitation. Conventional methods for measuring joint kinematics typically allow only static measurements performed by specially trained therapists. This paper introduces skin-conformal, wearable wireless systems designed to continuously and accurately capture the angles of target joints, specifically joints within the hand and wrist. Supported by a computer vision-based calibration protocol run on a smart device, these magnetometer-based standalone systems provide patients and clinicians with continuous, real-time data on joint angles and ranges of motion through an intuitive graphical interface. Human trials in healthy volunteers demonstrate the accuracy and precision of the electrogoniometry system, as well as its compatibility with simulated hand therapy. This study has also demonstrated that the electrogoniometry system is suitable for tracking complex and rapid movements and for deployment during occupational tasks where it could serve as a biofeedback device to warn against excessive and clinically contraindicated motion.

Impairment in an upper extremity, including the arm, forearm, wrist, and hand, can significantly reduce independence in activities of daily living as well as quality of life, while also incurring substantial financial costs. Although the causes of such impairments vary-ranging from stroke to spinal injury and trauma-ensuring optimal recovery through proper rehabilitation is crucial. Inadequate rehabilitation can lead to impaired recovery, often resulting in a permanent loss of range of motion (ROM) and motor skills. However, fewer than 55% of patients typically comply with their rehabilitation programs, missing the critical window for optimal recovery. Biofeedback that incorporates quantitative measurements of joint kinematics has proven to be a valuable tool for both patients and clinicians during rehabilitation, as it provides real-time biological information that would otherwise be inaccessible. This real-time data can enhance therapeutic outcomes by helping patients participate more effectively with their rehabilitation protocols, in both the supervised and unsupervised settings.

In nearly all traumatic conditions of the hand, injury and treatment are associated with joint stiffness and lost range of motion. Rehabilitation from the injured state, as such, is highly focused upon the restoration of maximal ROM. Clinical assessment of ROM is a fundamental parameter used to evaluate joint movement functionality, mobility, and rehabilitation progress. Standard approaches to measuring hand ROM encompass visual estimation, goniometry, composite finger flexion to the distal palmar crease, torque range of motion, and wiregrams. The most ubiquitous tool in clinical hand therapy is the hand-held analog goniometer. The accuracy and precision of this tool is inconsistent, and depends not only upon the type of goniometer used, but also upon the expertise of the therapist making the measurement. Additionally, traditional goniometry provides only static measurements, and continuous data cannot be gathered. This means that feedback is only offered at the beginning and end of a supervised therapy session, and no feedback is available during active exercise, or between sessions during execution of a home exercise program (HEP).

Existing strategies for continuous measurement of hand and wrist ROM that are both technically advanced and clinically sound include computer vision, wearable sensors utilizing strain or bending sensors and magnetometers, wrist motion sensing using electromyography or pressure sensor, and inertial measurement units. These advanced approaches, however, face practical challenges which limit adoption. These include occlusions and restricted workspace; the need for large training datasets; limited capabilities offering only gesture recognition rather than precise angle measurement; reliance on tethered systems with bulky benchtop electronics; the need for additional calibration equipment; and significant computational power requirements.

A comparison of various technologies that can measure joint angles is presented in Table 1, highlighting key characteristics such as sensor, targeted modality, uncertainty, calibration methods, connection type, and computing and data processing platform. These characteristics may serve as criteria for evaluating the feasibility of deploying these technologies in both in-clinic and at-home rehabilitation settings, where compatibility with current rehabilitation protocols is particularly important for enhancing therapeutic outcomes.

TABLE 1
Comparison of advanced technologies for goniometry of joint angles in hand.

| | | | | Calib | | |
|---|---|---|---|---|---|---|
| Sensor | Mod. | Error | Calib. | T. | Wired | Platform |
| Vision; | Angle; | <9.4°; user | ML | ◯ | ◯ | PC; one |
| Markerless | gesture | | | | | camera |
| Vision | Angle | ≈2.8°; | N/A | X | ◯ | PC; five |
| | | replica | | | | camera |
| Vision; | Angle | <12°; user | Re- | ◯ | ◯ | PC; MS |
| Markerless | | | viewer | | | Kinect |
| Vision; | Angle | <4°; user | N/A | X | ◯ | PC; ≥two |
| Markerless | | | | | | camera |
| Strain | Angle | <12°; user | Mo-cap; | ◯ | ◯ | PC |
| | | | per-use | | | |
| Strain | Angle; | <13.4°; | Mo-cap; | ◯ | ◯ | PC |
| | Gesture | user | per-use | | | |
| FBG | Angle | N/A | IMU; per- | ◯ | ◯ | PC; |
| | | | use | | | spectrometer |
| FBG | Angle | <3°; replica | N/A | N/A | ◯ | PC; |
| | | | | | | interrogator |
| Magnet | Position | <1 mm; | N/A | N/A | ◯ | PC; iter |
| | | unknown | | | | |
| Magnet | Angle; | NS | Mo-cap; | ◯ | ◯ | PC |
| | Position | | ML; one- | | | |
| | | | time | | | |
| EMG | Angle | ≈18.3°; | ML | ◯ | X | Smartphone |
| | | user | | | | |
| EMG | Angle | <15% ROM | Mo-cap; | ◯ | ◯ | PC |
| | | user | ML | | | |
| Pressure | Angle; | ≈0.11 | ML | NS | ◯ | PC |
| | gesture | RMSE; | | | | |
| | | user | | | | |
| IMU | Angle | ≈6°; user | per-use | X | ◯ | PC |
| IMU | Gesture | N/A | ML | N/A | X | PC |
| Magnet + | Angle | <5°; user | Vision; | X | X | Smartphone |
| Vision | | | per-use | | | |

Mod., Calib., and Calib T. stand for sensing modality, calibration methods, and additional equipment for calibration, respectively.
Platforms categorize computing and data acquisition or processing platforms.
Wired indicates whether the measurement system is wired or wireless.
*FBG: Fiber brag grating; EMG: electromyography; IMU: Inertia measurement unit; ML: machine learning; Mo-cap: motion capture system; PC: personal computer; iter: iteration solver; NS: not stated; Magnet + Vision = this study.

The ability to continuously track joint angles in the hand and wrist using a wireless, wearable system that conforms to the surroundings of a target joint would address such challenges in ROM biofeedback for rehabilitation. Reliable and instant reporting of this kinematic biofeedback to clinicians and patients, whether in-clinic or at-home physical therapy settings, benefits both parties and may enhance rehabilitation outcomes. Wireless, wearable electrogoniometry systems are described below that provide continuous, real-time measurements of joint angles in hand and wrist, displayed through a graphical user interface (GUI) on a smart device. The unique design of the device, equipped with optimized data flow, facilitates seamless integration of computer vision with a magnetometer, effectively overcoming obstacles commonly encountered in wearable goniometers, such as the need for motion capture systems for per-use calibration, being tethered to benchtop electronics, and significant computational power. Importantly, the calibration methodology developed in this work formulates the relationship between local orientation of a magnet and target joint angles in a simple polynomial function, thereby avoiding iterative solvers, machine learning, or kinematic modeling typically required in magnet-based tracking of hand motions (Table 2).

Additionally, the disclosed methodology customizes the calibration function for each use, enabling the electrogoniometry system to be highly adaptable across trials and users. Once calibrated, the system relies solely on magnetometer data, thus addressing typical limitations of vision-based system, such as occlusions and restricted workspace. Results from physical simulations and comparisons with two different ground truth methods-3D marker tracking under a multi-camera setup and 2D markerless tracking from fluoroscopy videos-demonstrate the favorable characteristics of the proposed systems, including high accuracy and precision (error <5°) which is clinically acceptable. As described below, human trials in ten healthy volunteers confirmed that the disclosed systems perform well in the setting of clinically relevant hand therapy exercises. The wearable devices are operated using only a standard smart device and requires no additional equipment for calibration. Furthermore, experiments with vigorous activities and daily tasks over the long term (7 hours) highlight the potential of the systems for applications beyond the primary clinical use.

TABLE 2
Comparison of magnet-based tracking
technologies for hand motion.

| Con- | | | | | | |
|---|---|---|---|---|---|---|
| fig. | Mod. | Method | Error | RT | Wired | Host |
| 1PM; | Position | magn field; | <1 mm | N/A | ◯ | PC |
| 4Mag | | particle | | | | |
| | | filter; iter; | | | | |
| | | kinematics | | | | |
| | | | <6.07 mm | X | ◯ | PC |
| | | | (ring); <8.35° | | | |
| | | | (ring) | | | |
| 2PM; | Ring | 2D | NS | ◯ | X | NS |
| | Ori. | | | | | |
| 1Mag | and Pos | projection | | | | |
| 3EM; | Finger- | magn field; | 0.95 | ◯ | ◯ | PC |
| 4Mag | tip | bandpass | mm(fixed); | | | |
| | Pos | filter | 1.33 mm | | | |
| | | | (random) | | | |
| 1PM; | Thumb | magn field; | 4.82 mm | ◯ | ◯ | PC |
| 2Mag | Pos; | iter | | | | |
| | tilt | | | | | |
| | angle | | | | | |
| 1PM; | Gesture | Approx; | <93% | NS | ◯ | PC |
| 9Mag | | homo trans | accuracy | | | |
| 1PM; | Hover | ML | 0.57 mm | ◯ | ◯ | PC |
| 3IMUs | Dist; | | (hover); | | | |
| 1FSR | Pressure | | 6.71% | | | |
| | | | (pressure) | | | |
| 1PM; | Finger- | AHRS | 5.5 mm | ◯ | ◯ | PC |
| 4IMUs | tip | | (static); <7.5 | | | |
| | Pos | | mm | | | |
| | | | (moving) | | | |
| 1PM; | Finger- | magn field; | 4.96 mm | ◯ | ◯ | PC |
| 1IMU | tip | particle | (2D); 8.6 mm | | | |
| | Pos to | filter | (3D) | | | |
| | Object | | | | | |
| 1PM; | Finger- | ML | 95% | ◯ | ◯ | PC |
| 1Mag | tip | | classification | | | |
| | Pos | | accuracy | | | |
| 5PM; | Finge- | Dipole | NS | NS | ◯ | PC |
| 6Mag | tip Pos | model; | | | | |
| | Joint | Kinematics | | | | |
| | angle | | | | | |
| 1PM; | Finger | magn field; | NS | NS | ◯ | PC |
| 18Mag | Pos and | iter | | | | |
| | Ori | | | | | |
| 1PM; | Joint | Polynomial | <5° | ◯ | X | SP |
| 1Mag; | angle | curve fitting | | | | |
| Vision | | | | | | |

Config., Mod., and RT stand for sensor configuration, sensing modality, and real-time operation, respectively.
Host categorizes a host system that completes data processing.
Wired indicates whether the measurement system is wired or wireless.
*PM: permanent magnet; EM: electromagnet; Mag: magnetometer; magn: magnetic; IMU: inertia measurement unit; FSR: force sensitive resistor; Ori: orientation; Pos: position; Dist: distance; iter: iteration solver; Approx: approximation; homo trans: homographic transformation; ML: machine learning; AHRS: attitude and heading reference system; NS: not stated; PC: personal computer; SP: smart phone; 1PM, 1Mag, Vision = this study.

### Results

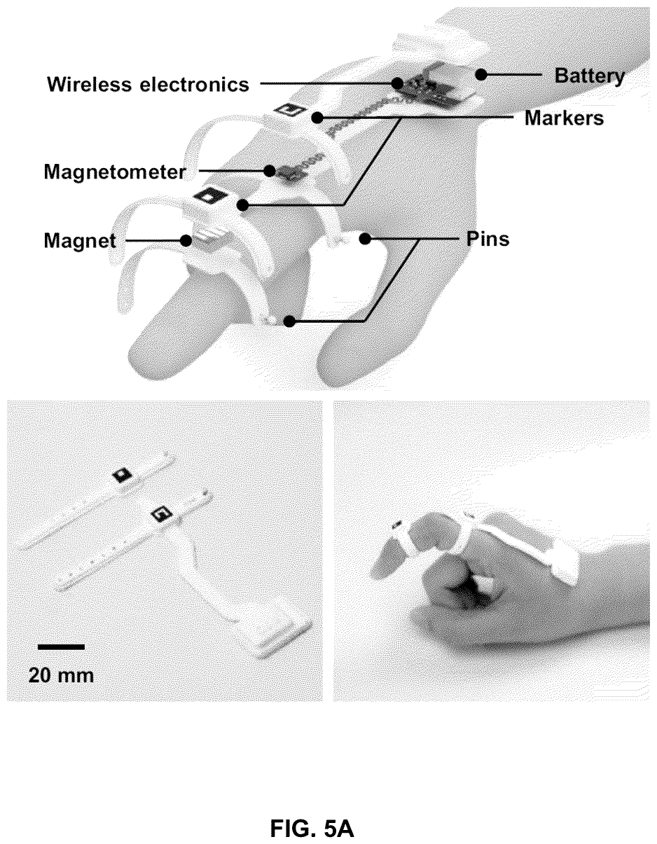

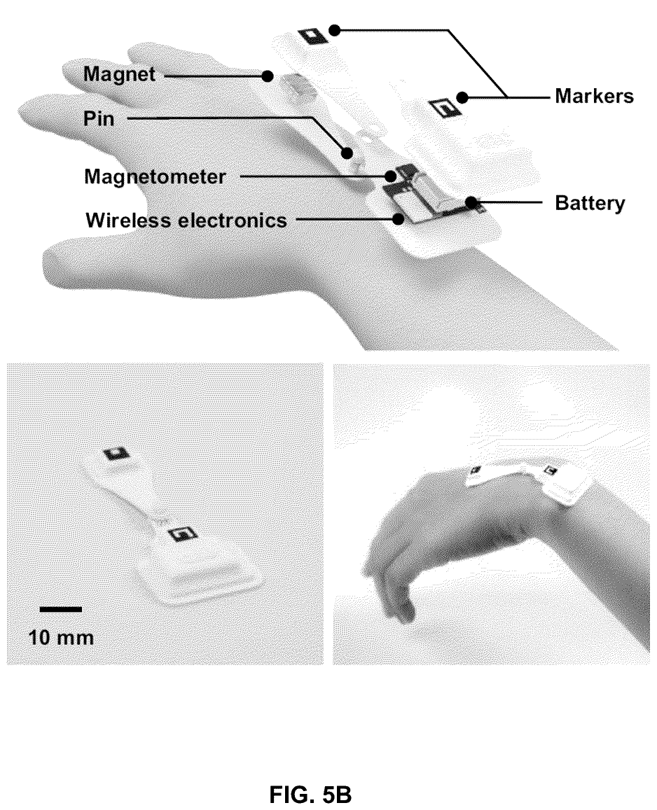

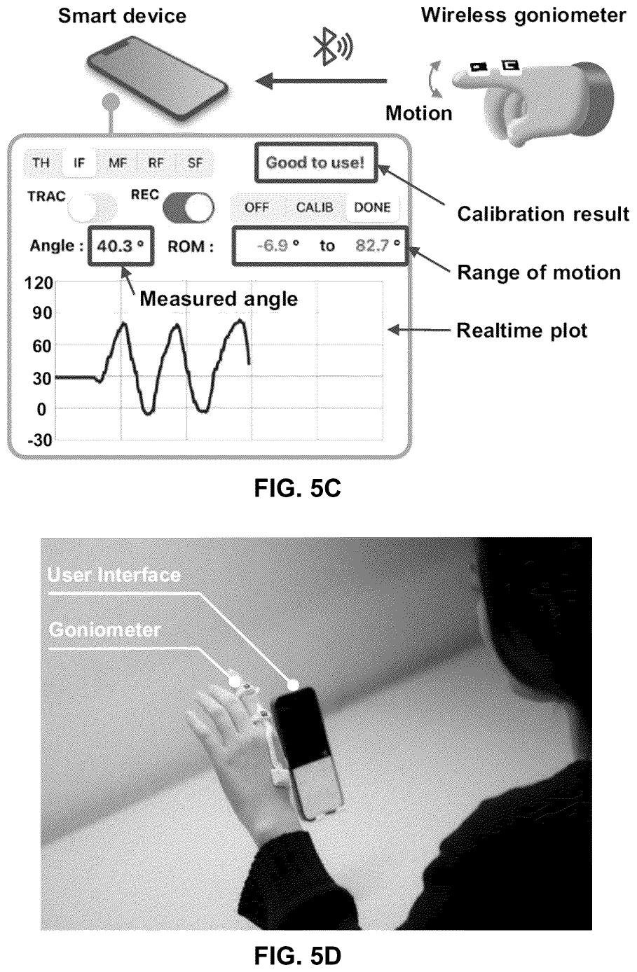

Herein, this study describes and validates the use of soft, wireless electrogoniometry systems for continuous monitoring of the joints in the fingers or wrists (FIG. 5A, FIG. 5B, FIG. 5C, FIG. 5D). The discrete design, soft mechanical properties, and wireless operational capabilities facilitate ergonomic wear without mechanically loading or constraining natural motions of targeted joints, a common issue with alternatives that use strain gauges. The electrogoniometry system is mounted on a user, with configurations for measuring the proximal interphalangeal (PIP) or wrist joints, respectively (FIG. 5A, FIG. 5B).

The finger goniometer (FG) features a strap band with a pin-and-hole design, allowing users to wear the device without the need for adhesives or additional straps (FIG. 5A), essential for many post-surgical patients. For the wrist goniometer (WG), a pin and a hole (FIG. 5B) allow two separate segments to be connected or disconnected as needed. The pin and hole also serve as a fiducial to facilitate initial positioning of the device on the wrist while the two segments are connected. Similar to the fulcrum of an analog goniometer used in standard clinical practice, this fiducial, placed over the capitate on the dorsal aspect of the wrist joint, ensures proper alignment of the magnet and magnetometer. The magnet is positioned on the dorsal aspect of the hand along the third metacarpal, while the magnetometer is placed on the distal forearm, aligning with the dorsal midline of the forearm.

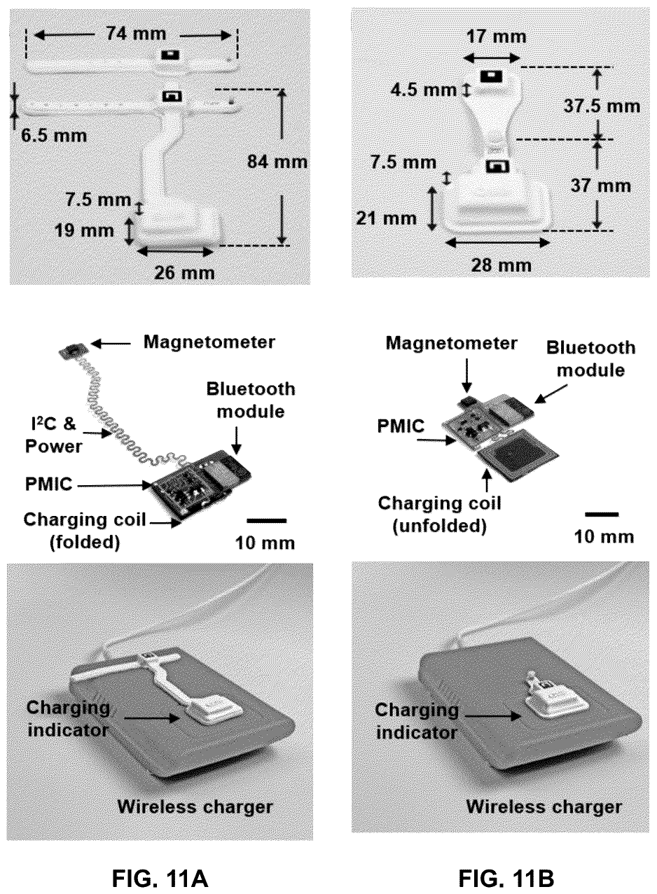

The schematic illustrations highlight additional features of the two segments of the FG and WG, both encapsulated in a soft silicone enclosure (FIG. 5A, FIG. 5B). A thin, flexible printed circuit board (fPCB) and a 3.7-V lithium polymer battery (45 mAh; dimensions: 15 mm length, 12 mm width, and 4 mm thickness) encased in medical-grade silicone shells (thickness: 0.5 mm; Silbione RTV 4420, Elkem) form one segment. The second segment includes a stack of four rectangular neodymium magnets (grade N50; dimensions: 6 mm length, 3 mm width, and 2 mm thickness) that form a cuboid structure with a height of 3 mm that produces a strong magnetic field in a low-profile design. Two different architectures in these segments define distinct electrogoniometry systems, the FG and WG, each tailored to different joints using respective configurations and dimensions (FIG. 11A, FIG. 11B).

The enclosures of the FG and WG function not only to isolate the electronics from the environment but also to facilitate joint angle measurements using embedded fiducial markers (i.e., ArUco markers). Patterns of silicone formed from white and black dye define high-contrast ArUco markers (FIG. 5A, FIG. 5B), The ArUco markers are provided to enhance the accuracy and reliability of joint angle measurements using computer vision for purposes of calibration using a smart device (iPhone 12 Pro, Apple Inc.).

The fPCBs (FIG. 5A, FIG. 5B, FIG. 11A, FIG. 11B) support and interconnect a collection of electronic components, including a magnetometer (MLX90393, Melexis), a power management integrated circuit (PMIC) with a wireless charging coil, and a Bluetooth Low Energy (BLE) system on a chip (SoC; ISP-1807, Insight SIP). The PMIC and foldable charging coil (FIG. 11A, FIG. 11B) facilitate wireless charging using near-field communication standards.

The magnetometer obtains digital measurements of magnetic field strength induced by the neodymium magnets of the second segment; magnetic field strength is evaluated along three axes and are indicative of joint angles. The magnetometer protrudes from the main fPCB body to align with an ArUco marker. For the FG, the magnetometer connects to the main body by a serpentine wire that bypasses the non-targeting joint. This design reduces the sensitivity of the goniometer to motions of non-targeted joints and allows the main body to be placed on the back of the hand.

A real-time GUI displays quantitative information on joint angles at 25-ms intervals. This interval is sufficiently fast to capture natural kinematics of the finger and wrist (FIG. 5C). Data communication between the smart device and the BLE SoC utilizes standard BLE protocols, ensuring smooth transmission of magnetometer data along with their timestamps. Along with real-time data, the GUI estimates and continuously updates minimum and maximum angles during use, to track the range of motion. Moreover, the GUI, integrated with the Open Source Computer Vision Library (OpenCV), accurately measures joint angles by tracking ArUco markers via the camera images obtained using the smart device. The information from the imaging of the ArUco markers is analyzed to determine joint motions used to calibrate the magnetometer responses to joint motions. The wearable goniometer systems described herein are suitable for use in both clinical and home settings (FIG. 5D).

Initial Calibration with Computer Vision and Routine Use

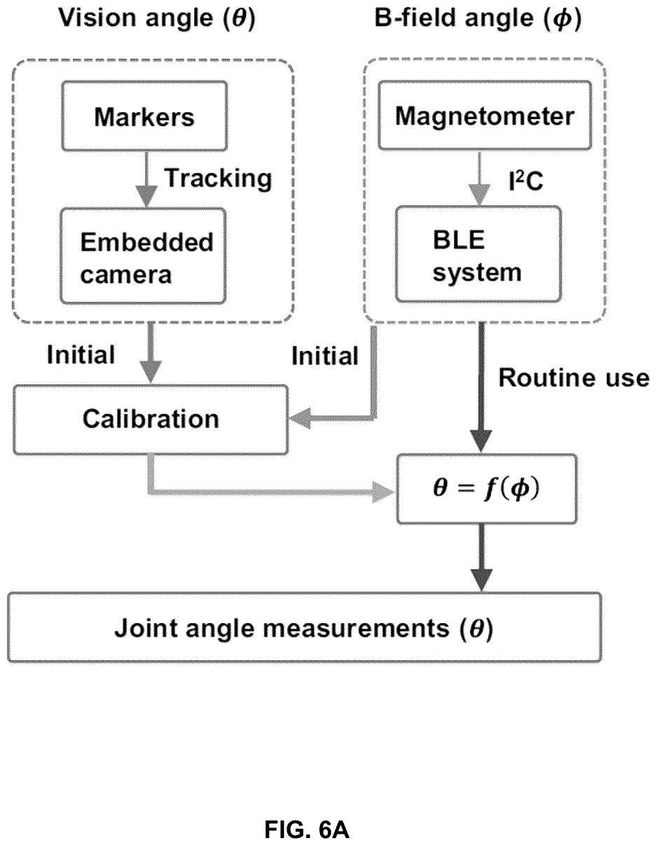

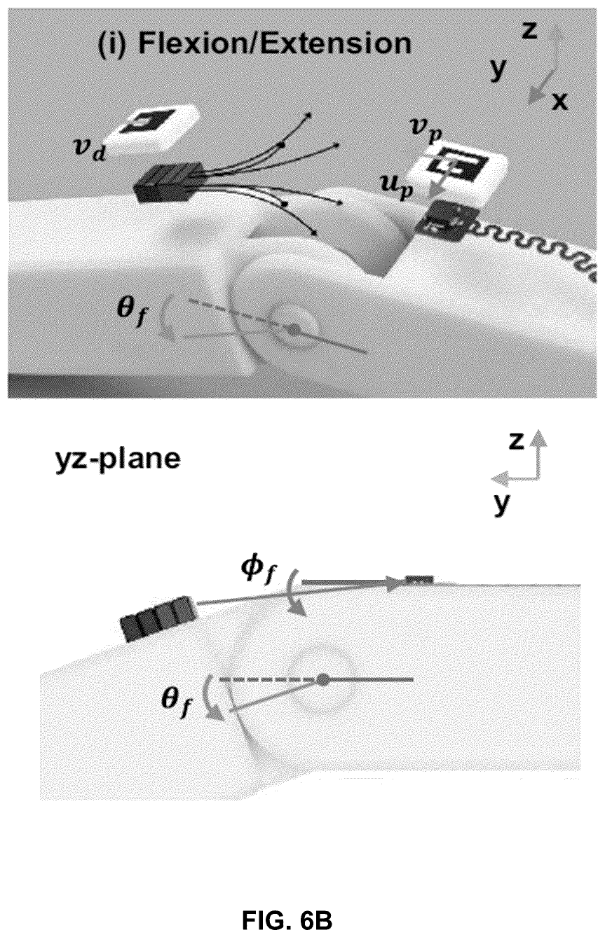

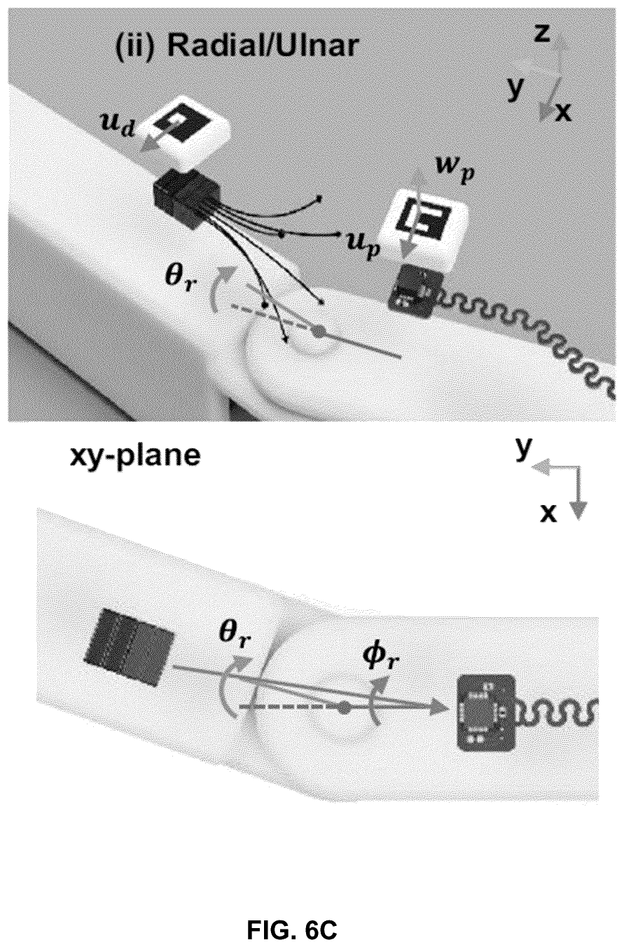

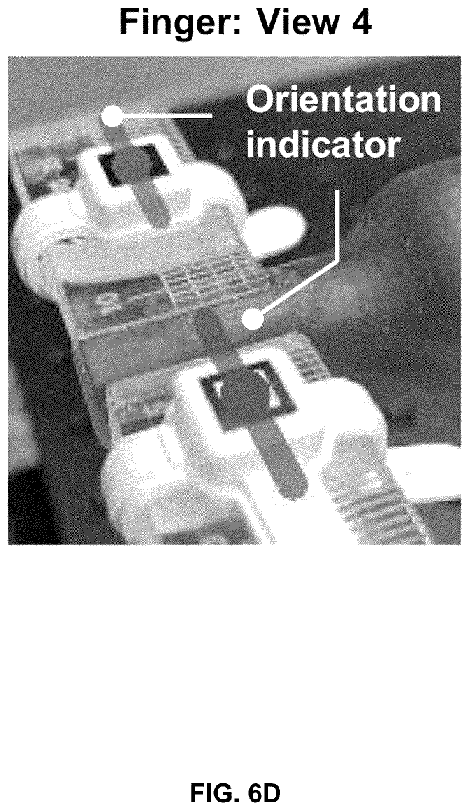

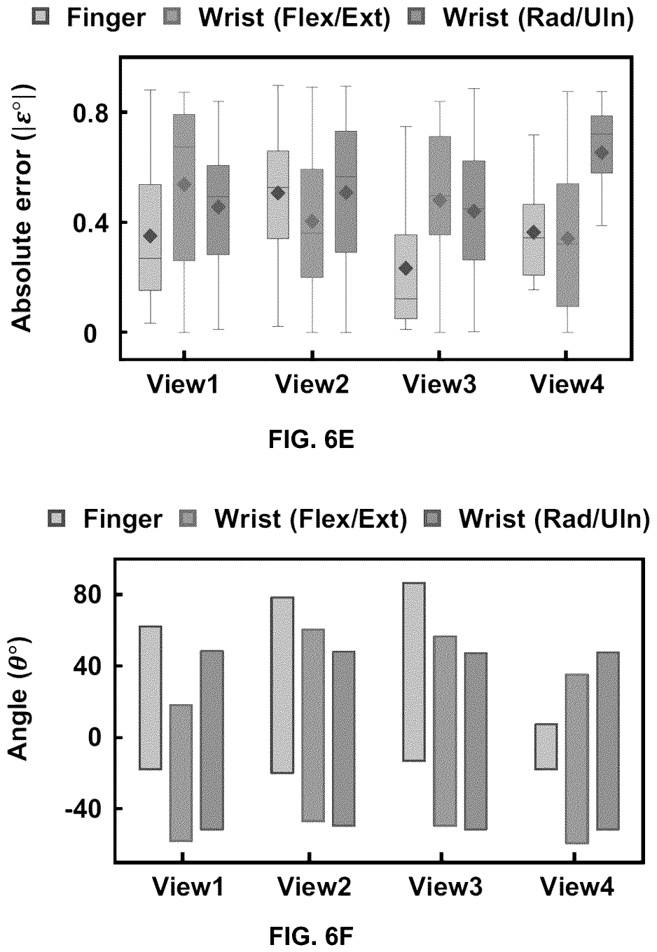

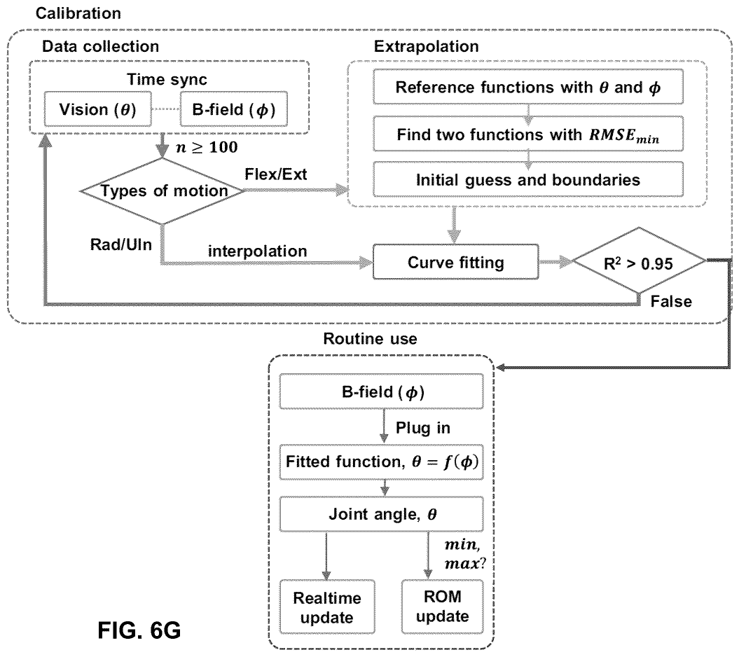

The procedures for calibration and subsequent routine use are detailed in FIG. 6A. The calibration obtains a calibration relationship between the magnetometer readings and the joint angles determined from the computer vision system over a range of joint angles. During routine use, only the magnetometer reading data is used and the calibration relationship is used to transform the magnetometer readings into estimated joint angles. The magnetometer transmits changes in magnetic field strengths along each of three axes (Bᵢ, where i=x, y and z) in a 3D Cartesian coordinate system to the BLE SoC at 30 Hz using the Inter-Integrated Circuit (I²C) protocol. These Bᵢ values facilitate the calculation of relative angles of the magnet cuboid to the magnetometer by using two Bᵢ on a targeting plane (FIG. 6B, FIG. 6C). For example, flexion/extension (flex/ext) motions occur in the yz-plane, and the angle resulting from these spatial motions can be defined as φ_{f}=a tan(B_{z}/B_{y}). Similarly, Bₓ and B_{y} on the xy-plane determine the angle from radial/ulnar (rad/uln) motions as φᵣ=a tan(Bₓ/B_{y}).

Even though φ changes along with changes in joint angles (θ), φ cannot directly represent θ because the center of rotation for φ is at the magnetometer, whereas the joint itself serves as the center of rotation for θ. The initial calibration step effectively addresses this limitation by establishing a relationship between θ and φ as a function of φ. The computer vision system with ArUco marker provides estimates of θ during the calibration step. Utilizing the embedded camera with OpenCV library and SceneKit in a custom app, the smart device tracks the positions and orientations of the two ArUco markers at 60 frames per second (fps). Similar to the existing orient-x-based method, orientation vectors of the markers on distal and proximal segments facilitate joint angle estimations for each motion. For instance, Of for flexlext motions can be expressed (FIG. 6B):

$${θ}_{f} = signum ( ( {v}_{p} \times {v}_{d} ) \times {u}_{p} ) {cos}^{- 1} ( {v}_{p} \times {v}_{d} ) \qquad ( 1 )$$

where the subscripts d and p represent distal and proximal, respectively. For rad/uln motions, x-axial orientation vectors (uₚ and u_{d}) and a z-axial orientation vector at the proximal (wₚ) can yield estimates for θᵣ.

$${θ}_{r} = signum ( ( {u}_{p} \times {u}_{d} ) \times {w}_{p} ) {cos}^{- 1} ( {v}_{p} \times {v}_{d} ) \qquad ( 2 )$$

Motions of targeted joints can be modeled as one degree-of-freedom (DOF) rotations. As such, this strategy based on axial orientation vectors effectively estimates joint motions.

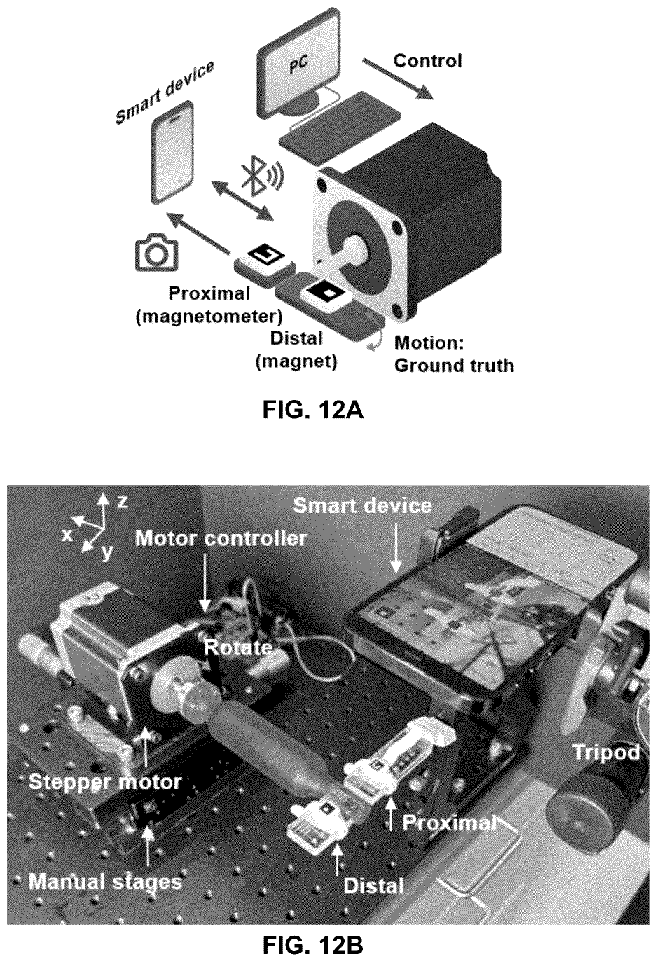

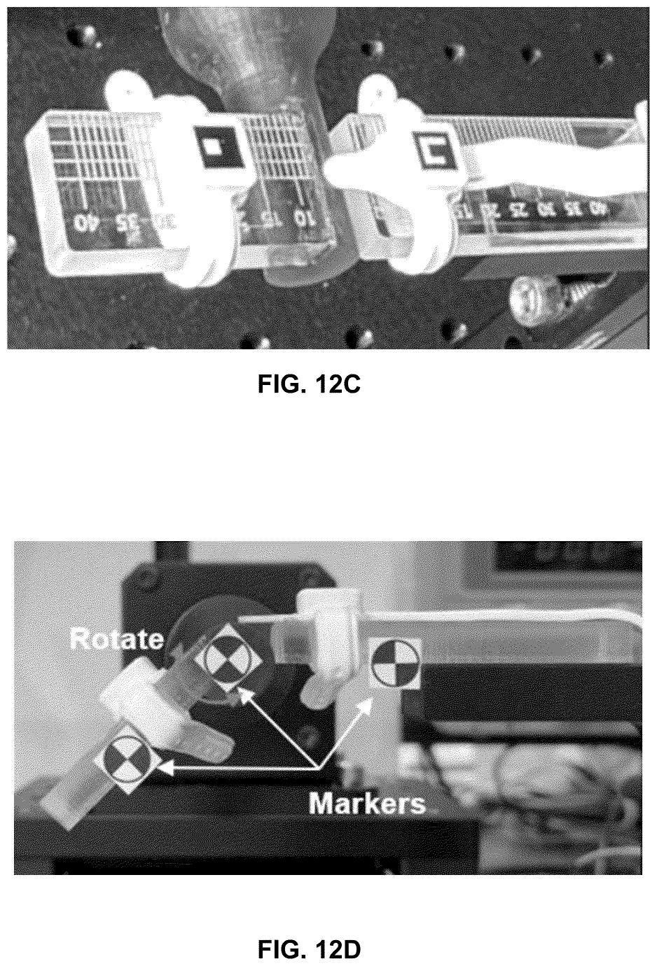

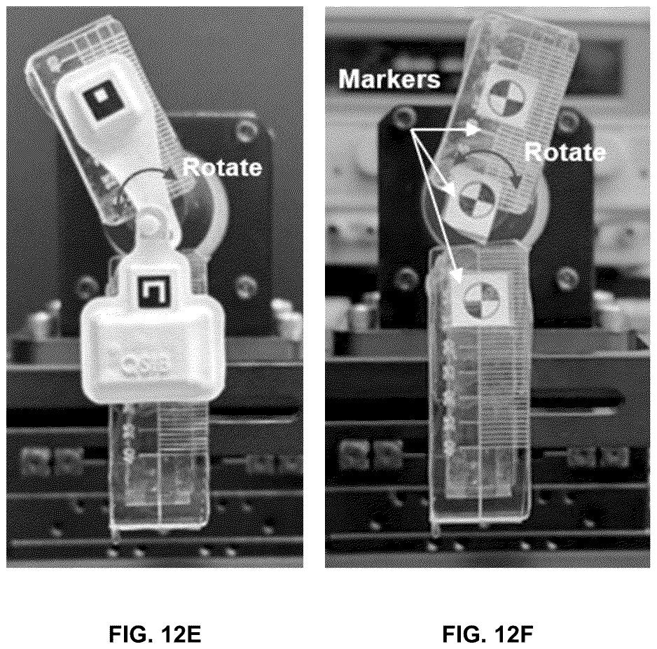

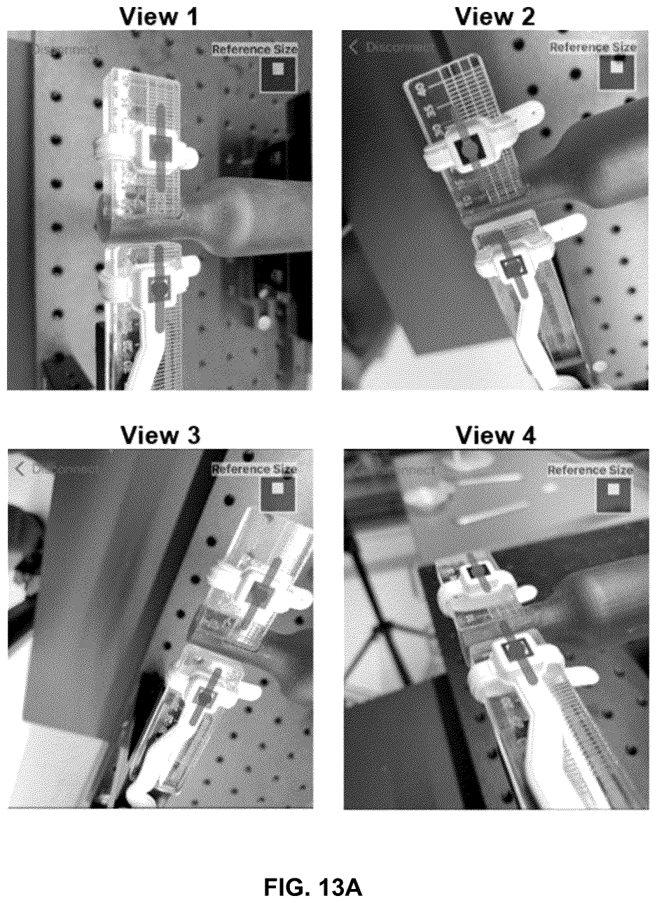

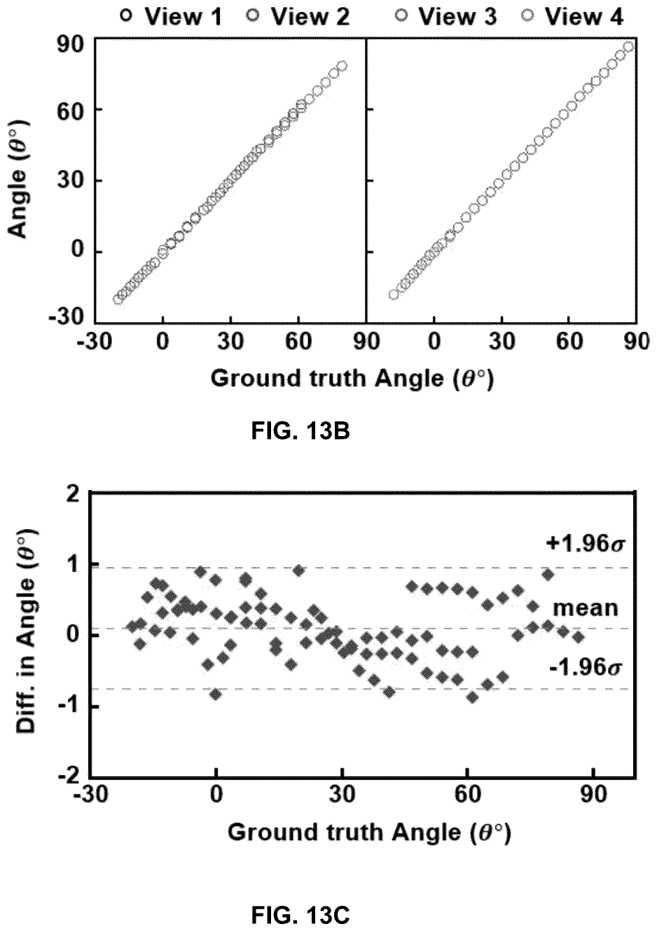

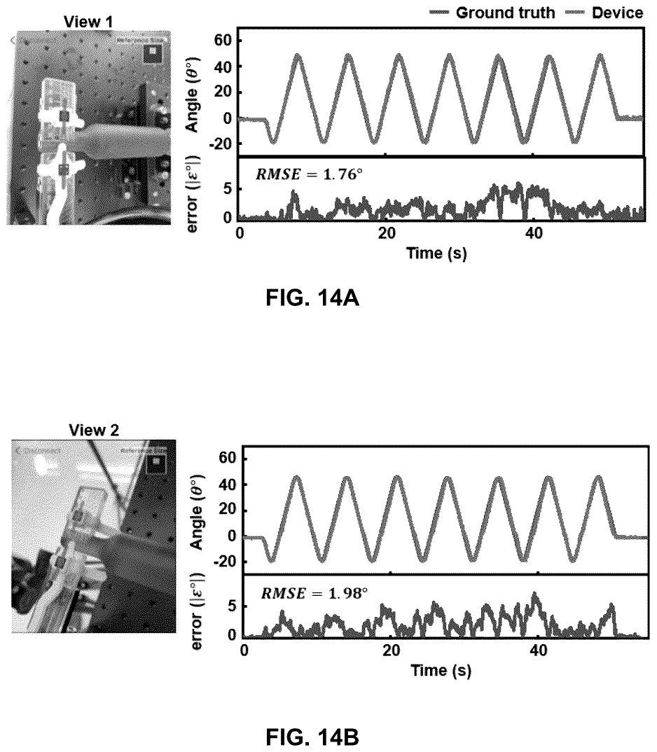

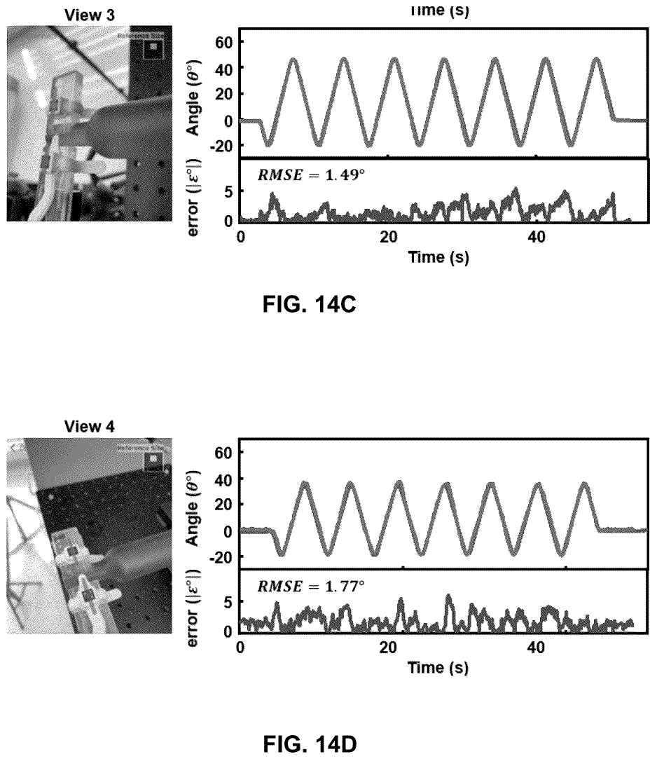

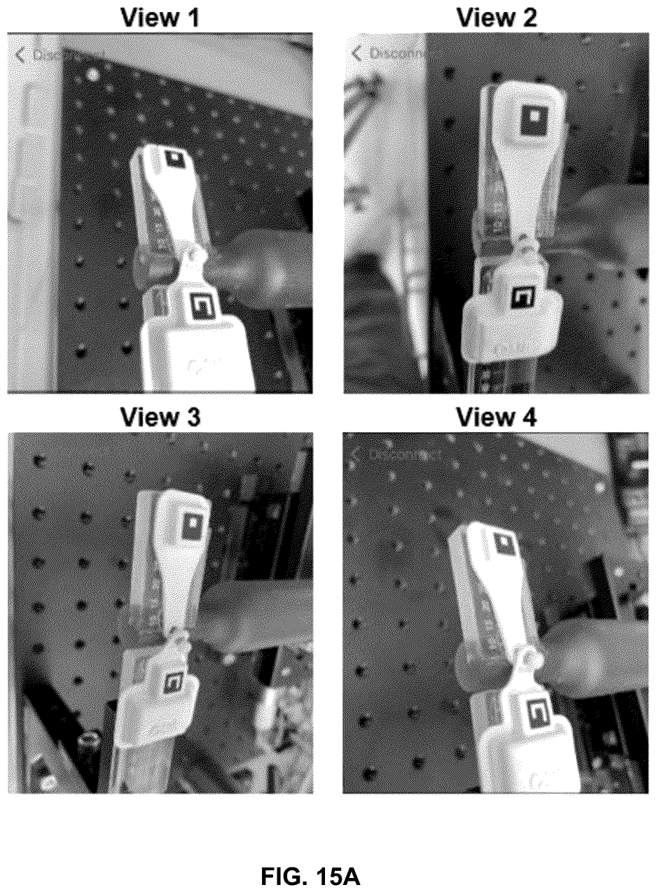

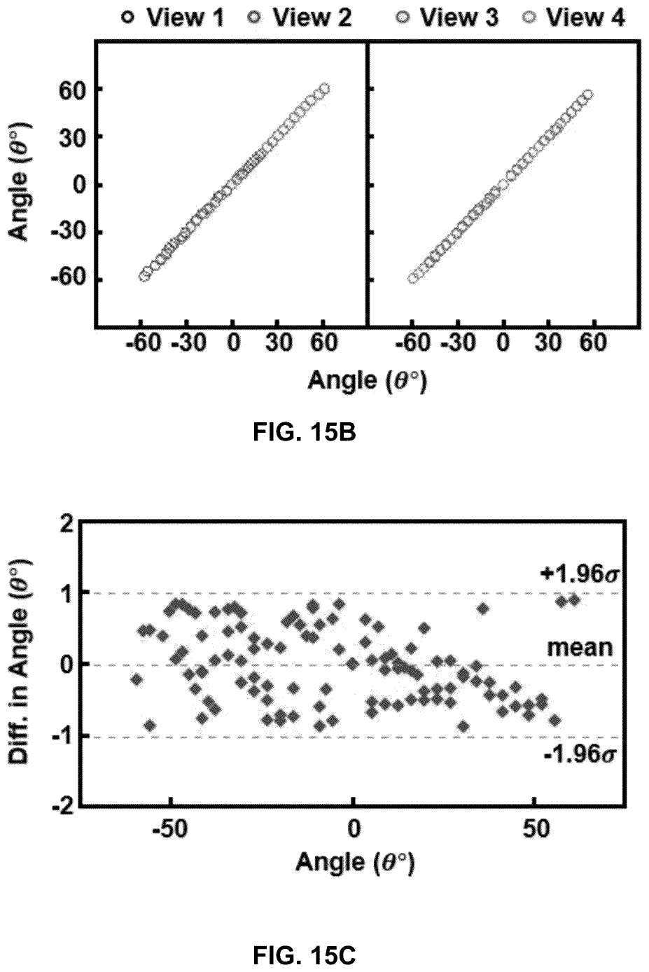

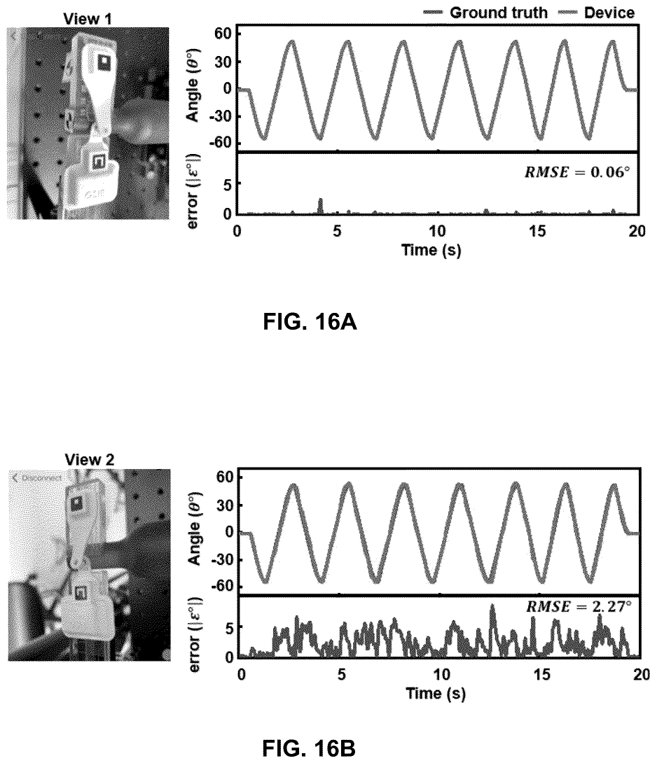

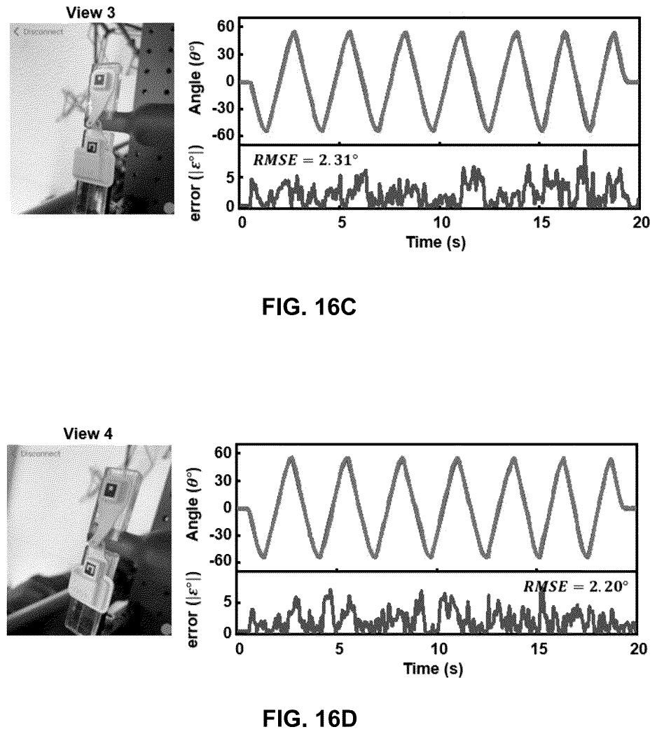

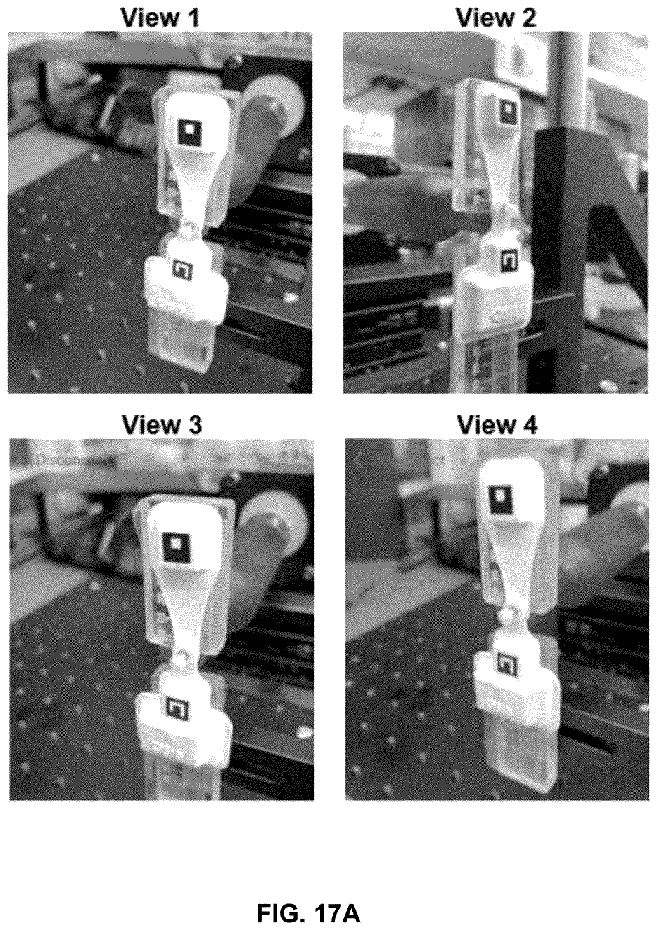

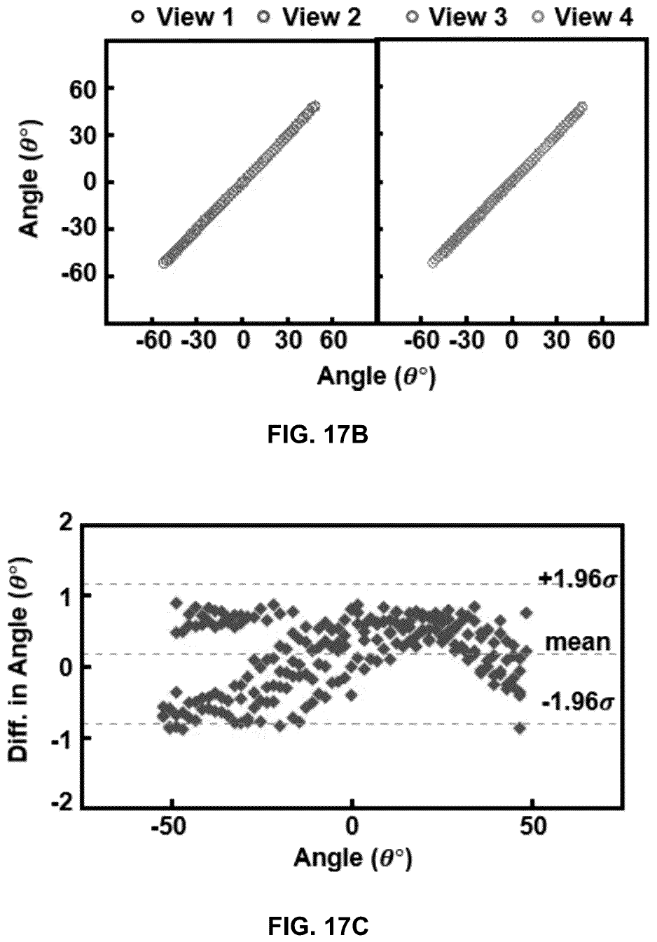

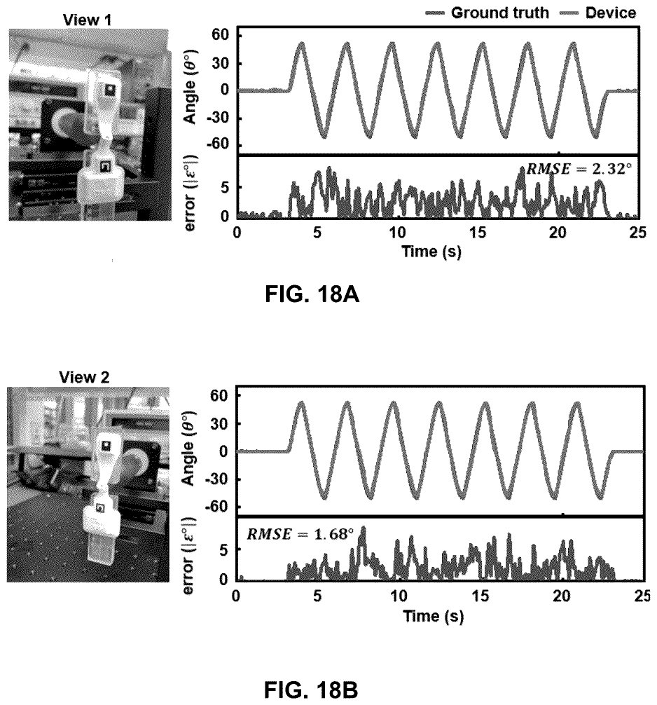

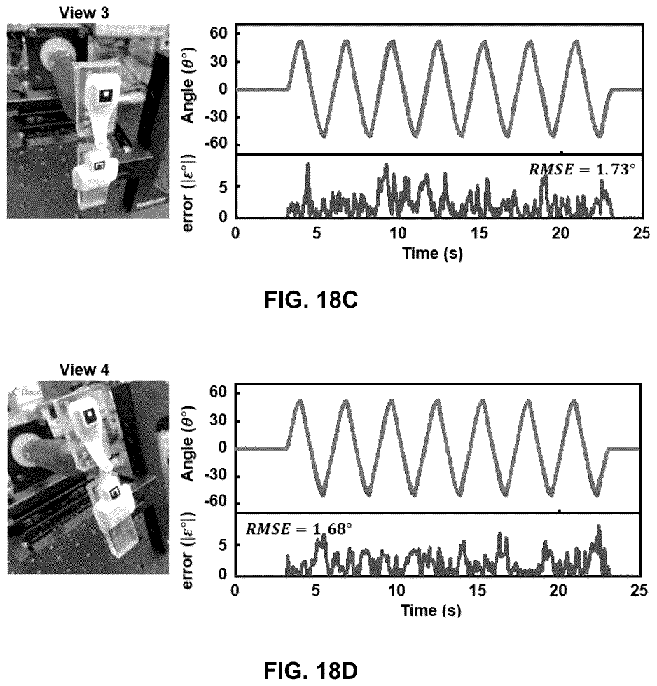

The screen of a smart device tracks ArUco markers from a specific camera angle (FIG. 6D). Orientation indicators on the markers inform the user about the status of the marker tracking. Absolute errors (FIG. 6E) between measured angles and applied angles from a motorized stage (FIG. 12A, FIG. 12B) are less than 0.9° across four camera angles for both flex/ext and rad/uln motions. Under these quasi-static conditions, the computer vision system accurately measures angles with errors less than 1.2° within a 95% confidence interval (FIG. 13C, FIG. 15C, FIG. 17C). Along with this high accuracy in angle measurements under quasi-static loads, root mean squared errors (RMSEs) of less than 2.3° for measured angles compared to applied cyclic loadings (FIG. 14A, FIG. 14B, FIG. 14C, FIG. 14D, FIG. 16A, FIG. 16B, FIG. 16C, FIG. 16D, FIG. 18A, FIG. 18B, FIG. 18C, FIG. 18D) further confirm the ability of the computer vision system to serve as ground truth for calibrating magnetometer responses to joint motions.

The initial calibration, followed by routine use is detailed in FIG. 6G. A smart device simultaneously collects angles from the computer vision system (θ) and the magnetometer (φ), synchronizing these data to accommodate their asynchronous sampling. After collecting over 100 samples of both θ and φ, the device implements two types of curve fitting-interpolation for rad/uln motions and extrapolation for flex/ext motions-using the MPFIT library. A single camera can capture the full range of rad/uln angles, thus making interpolation suitable for data from this motion (FIG. 6F). Flex/ext angles, however, exceed the capture range of a single camera angle, thus necessitating extrapolation to reconstruct the full range of motion from the limited calibration data.

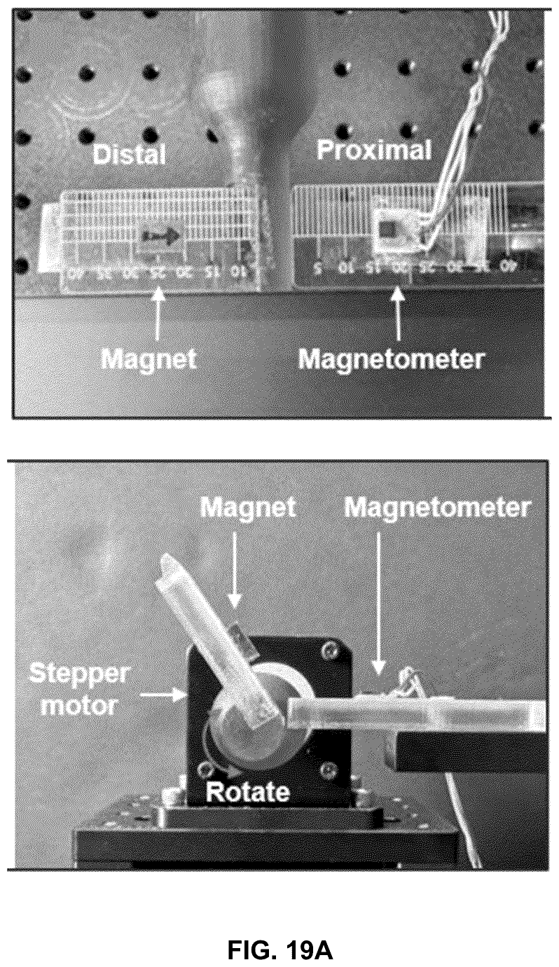

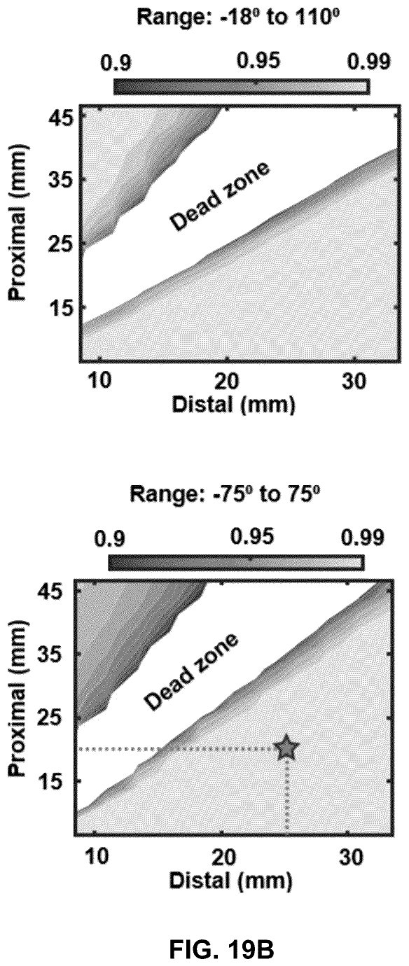

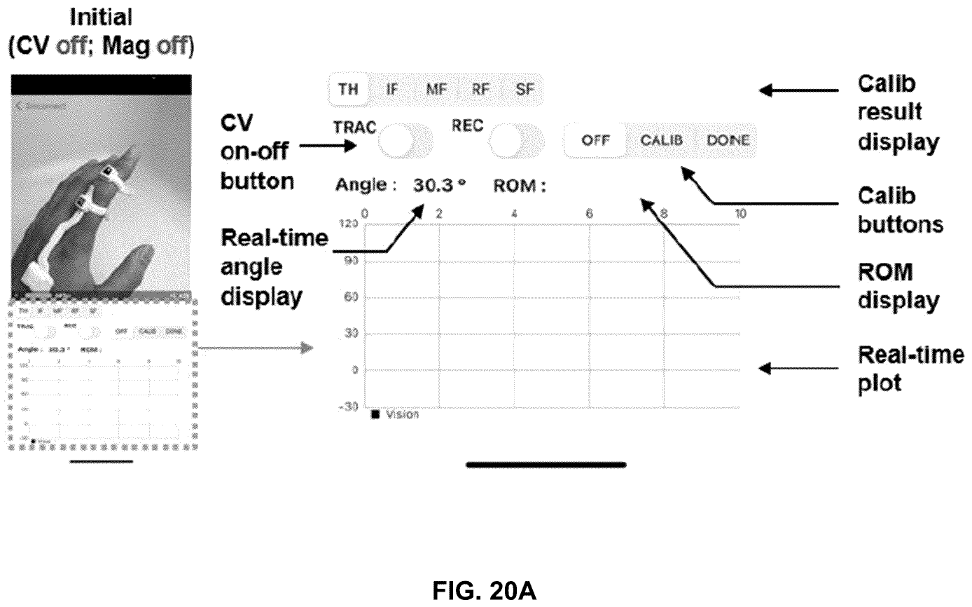

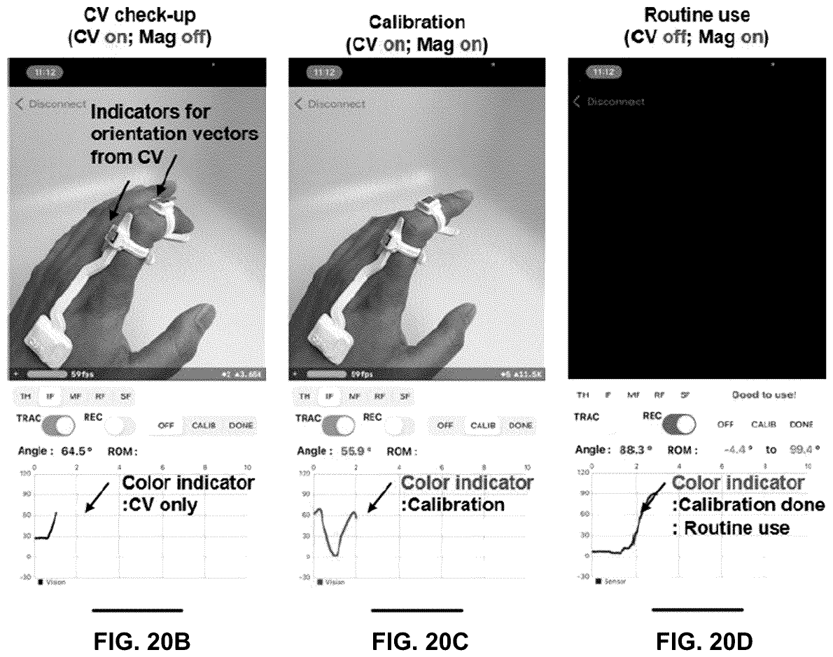

In the Extrapolation block (FIG. 6G), three steps are included to mitigate any intrinsic uncertainty in this process. Initially, the sampled φ and θ are examined using 120 reference functions, θ=f(φ). These functions, obtained from experiments that explore possible locations of the magnet cuboid and magnetometer (FIG. 19A, FIG. 19B), have R² values close to 0.99 and encompass the expected ROM of the hand digit PIP, IP, and wrist joint (Tables 3 and 4). The RMSE of θ to outputs from these functions allows selection of two functions with the minimum RMSE, and their coefficients establish an initial guess and boundaries for curve fitting. The FG employs extrapolation curve fitting, while the WG utilizes both interpolation and extrapolation, depending on the types of motion. The R² value from the curve fitting determines whether to proceed with the routine use or to recalibrate. Once the fitted function meets the criterion (R²>0.95), the system estimates joint angles using magnetometer data and the fitted function, as shown in the Routine use block (FIG. 6G). Details on this calibration procedure and its implementation in the GUI are shown illustrated in FIG. 20A, FIG. 20B, FIG. 20C, and FIG. 20D.

TABLE 3
Range of motion of proximal interphalangeal joints in fingers and
interphalangeal joint in thumb under flexion/extension motions.

| Finger | Thumb | Index | Middle | Ring | Little |
|---|---|---|---|---|---|
| Flexion | 74.8 ± 13.2° | 99.4 ± 13.5° | 101.9 ± 11.6° | 103.3 ± 7.9° | 98.9 ± 13.4° |
| Extension | −7.2 ± 4.2° | −5.5 ± 7.1° | −6 ± 8° | −5.6 ± 7.3° | −5.4 ± 7° |

TABLE 4
Range of motion of wrist joint under flection/extension
and radial/ulnar motions.

| Motion | Flexion | Extension | Radial | Ulnar |
|---|---|---|---|---|
| Angle | 72.4 ± 7.2° | −67 ± 6.2° | 19.5 ± 2° | 38.3 ± 3.9° |

### Responses of the Electrogoniometry Systems Under Benchtop Experimental Conditions

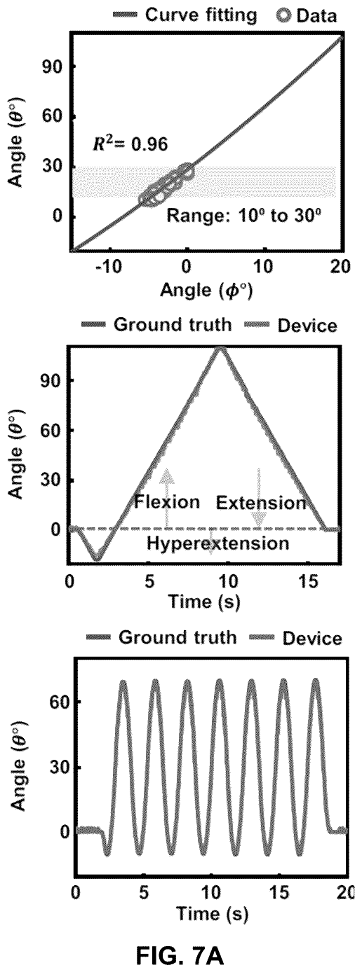

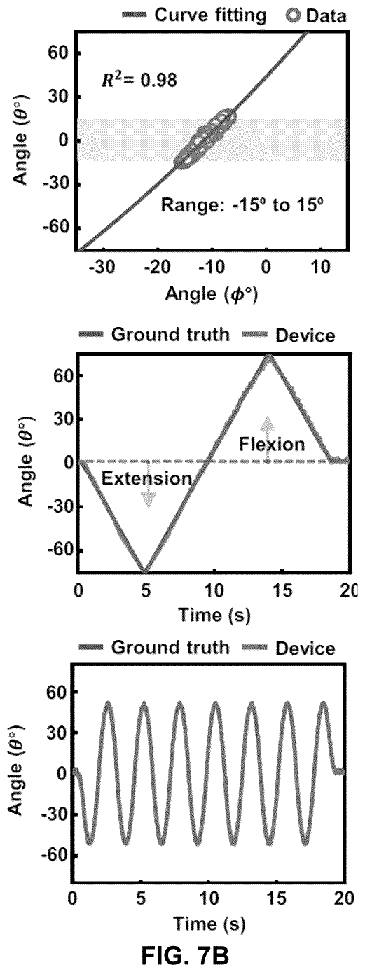

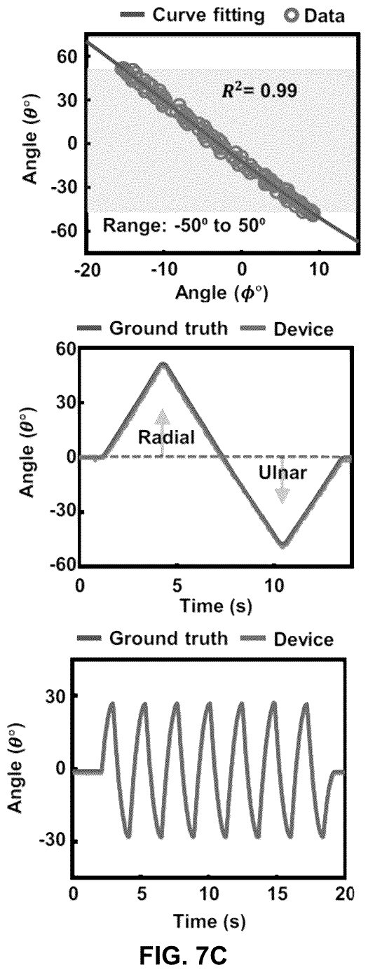

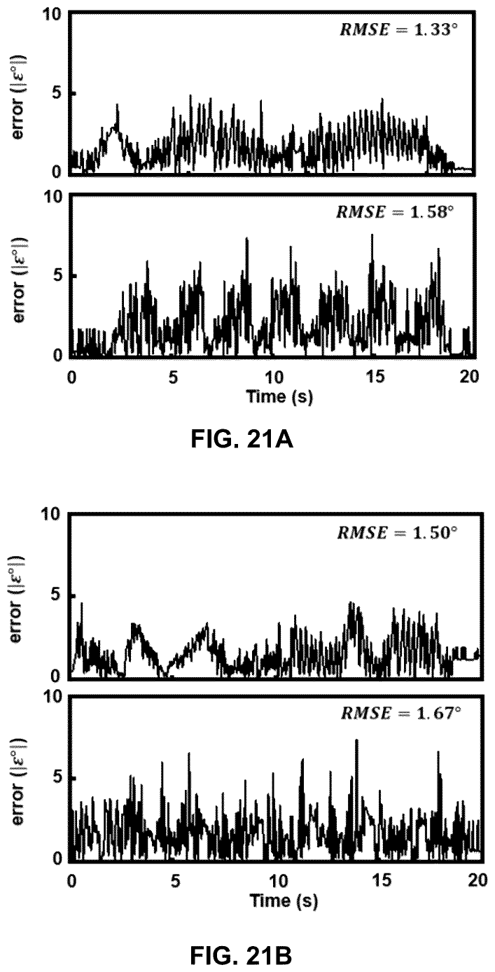

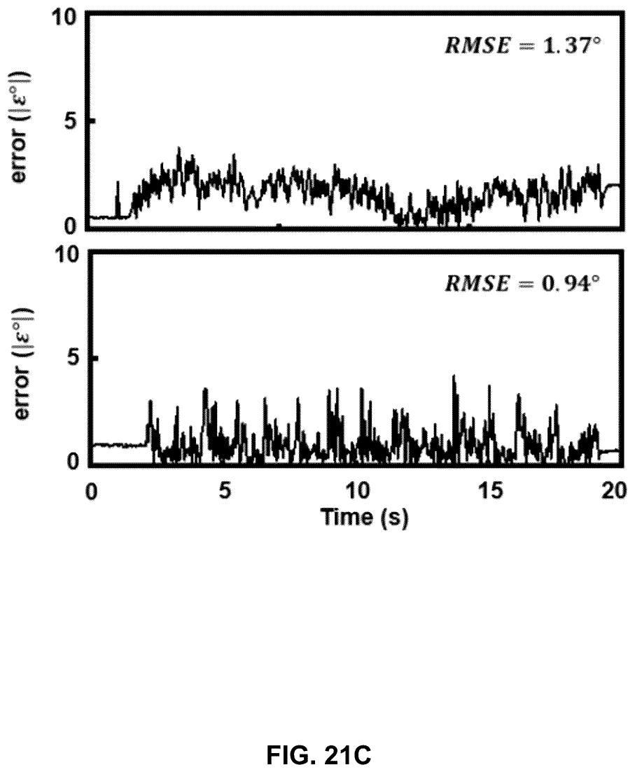

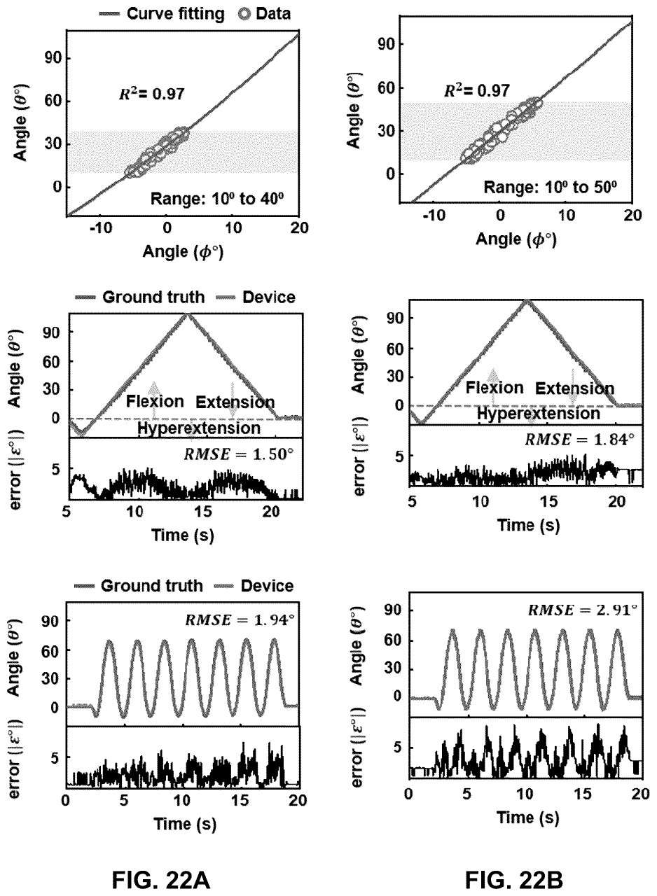

The responses of the electrogoniometry systems under a benchtop experimental setup are shown in FIG. 7A, FIG. 7B, FIG. 7C, and FIG. 12A. These experiments evaluated the effect of the range of calibration data on extrapolation accuracy, and the accuracy and repeatability of measurements. The finger goniometer (FG) effectively responded to low-rate angular loads (17.6°/s) across the expected full range of motion of the PIP and IP joint (from −18° to) 110° using only a 20° range of calibration input (from 10° to 30°; FIG. 7A). A low RMSE (1.3°) of the angles measured by the FG confirms its high measurement accuracy (FIG. 21A). Additionally, the FG shows highly repeatable responses to cyclic loadings with a higher average rate (from −9.9° to 72° at 64.2°/s), where the RMSE to ground truth is 1.6° (FIG. 21A). Moreover, RMSEs of less than 2.9° in measurements with increased ranges of calibration data (30° and 40°; FIG. 22A, FIG. 22B) suggest that responses of the FG are minimally sensitive to the range within the test window.

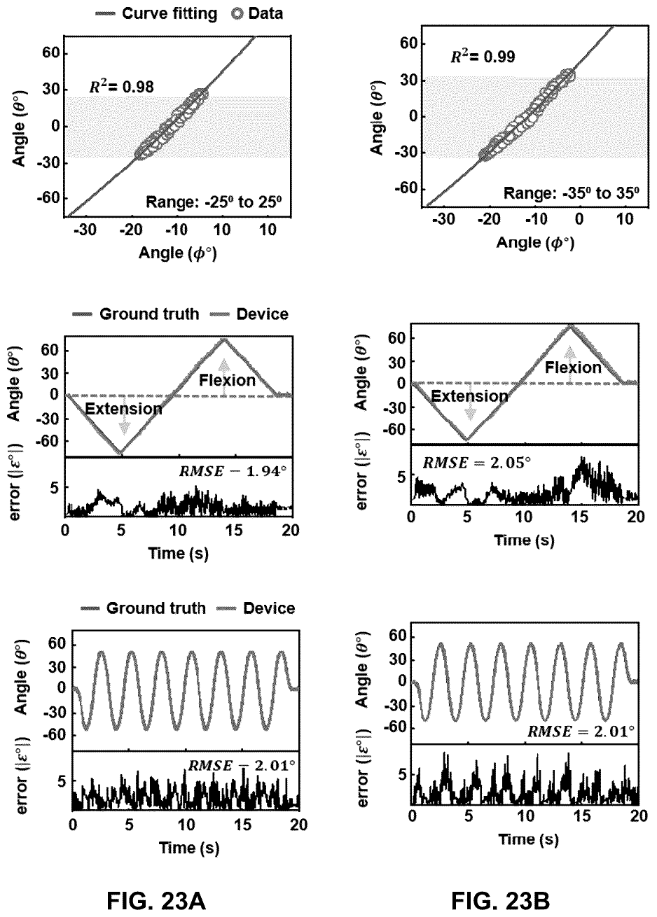

The wrist goniometer (WG) measures flex/ext angles with high accuracy and precision under similar experimental conditions (FIG. 7B). Similar to the FG, the WG demonstrates low RMSEs to ground truth (<2°) under angular loads across the expected full range of motion (+74.7° at 17.6°/s) and cycles (+49.4° at 71.4°/s), regardless of the range of calibration data (FIG. 23A, FIG. 23B). FIG. 7C depicts rad/uln angles measured using the WG. The responses of the WG to rad/uln motions (full motion: +51.3° at 17.6°/s; cycle: +27.9° at 46.1°/s) exhibit improved accuracy and repeatability, likely due to an improved fitted function achieved through interpolation. The results from both flex/ext and rad/uln loads indicate that the WG can measure both types of motion with high precision, and that the ranges of calibration data also minimally affect its performance within the test window, similar to the FG.

The RMSEs tend to increase along with larger ranges of motion and higher angular load rates. This trend does not stem from defects in the electrogoniometry systems but rather from limitations in the error analysis performed in this study. Errors between two asynchronously sampled time-series data sets can be quantified by first resampling the data from the ground truth (60 Hz) to match the lower sampling rate of the device (25 Hz). This resampling, along with potential time mismatches, leads to higher errors under more dynamic conditions. Outliers in absolute errors observed during transitions (FIG. 21A, FIG. 21B, FIG. 21C, FIG. 22A, FIG. 22B, FIG. 23A, FIG. 23B). The remaining sections utilize this error analysis method to quantify discrepancies between the angles measured by the systems and the ground truth.

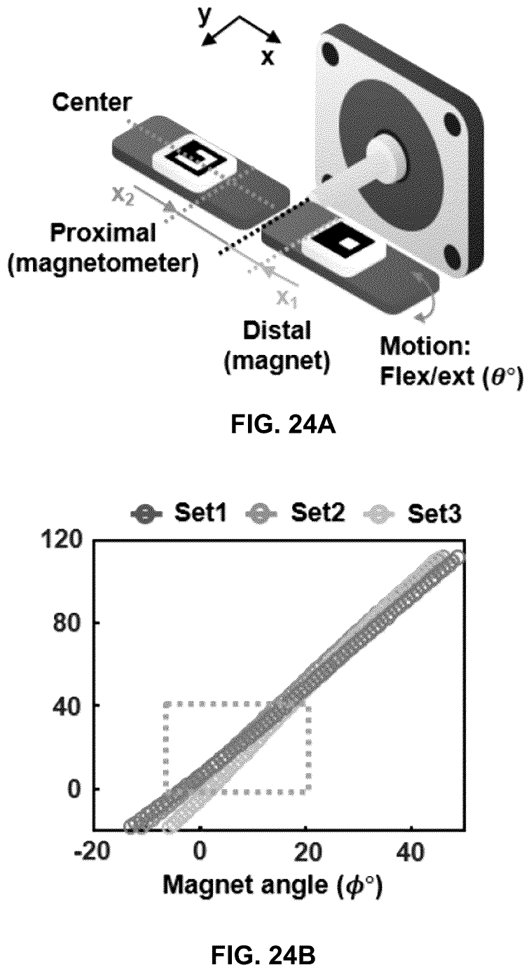

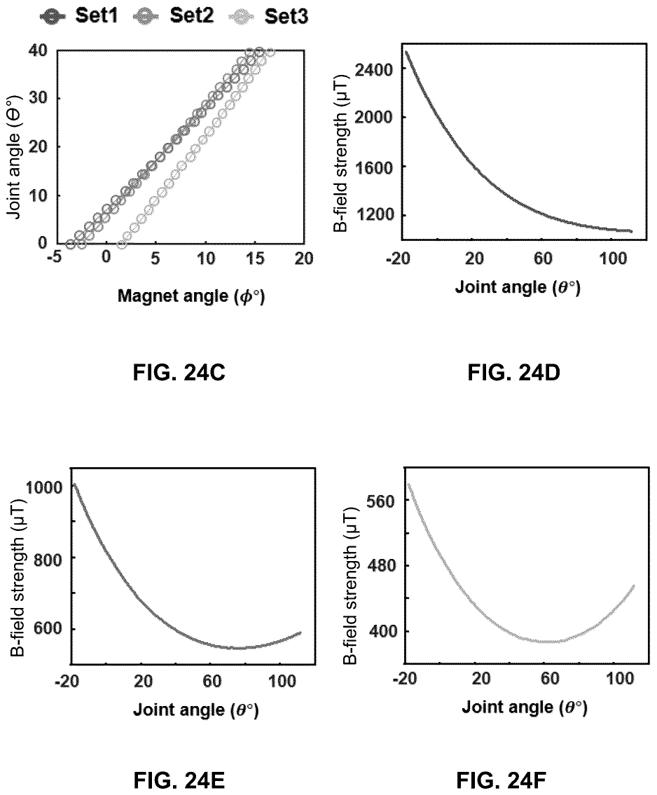

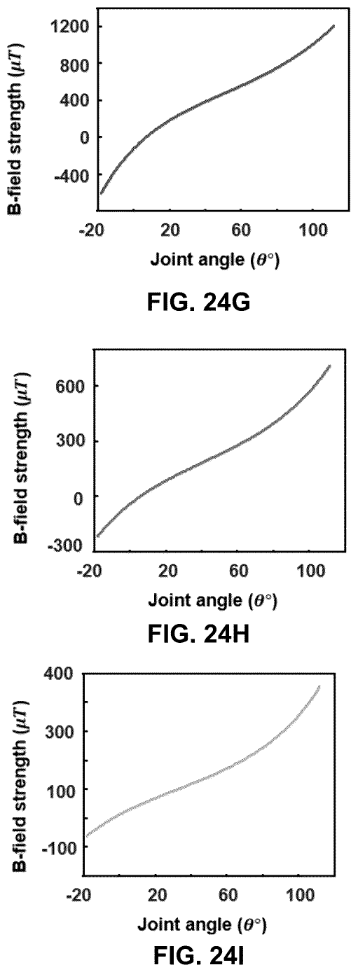

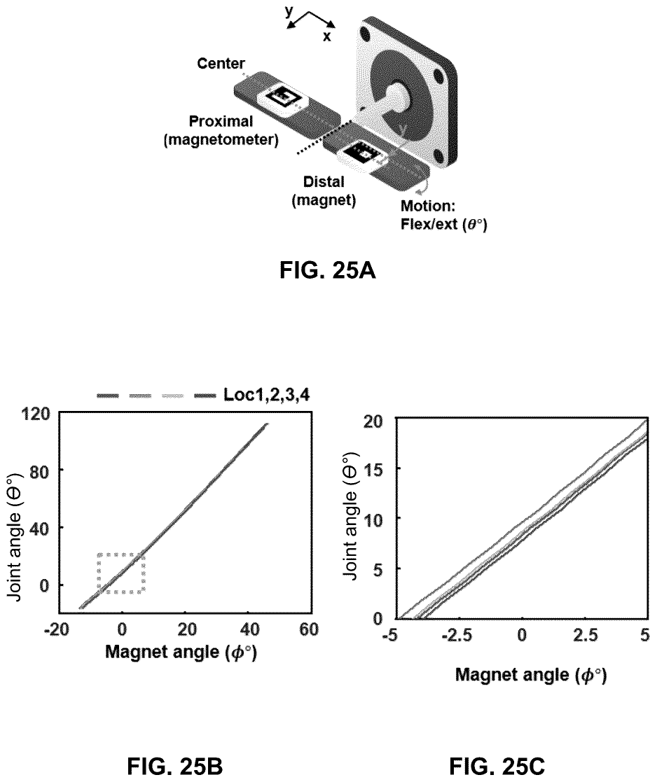

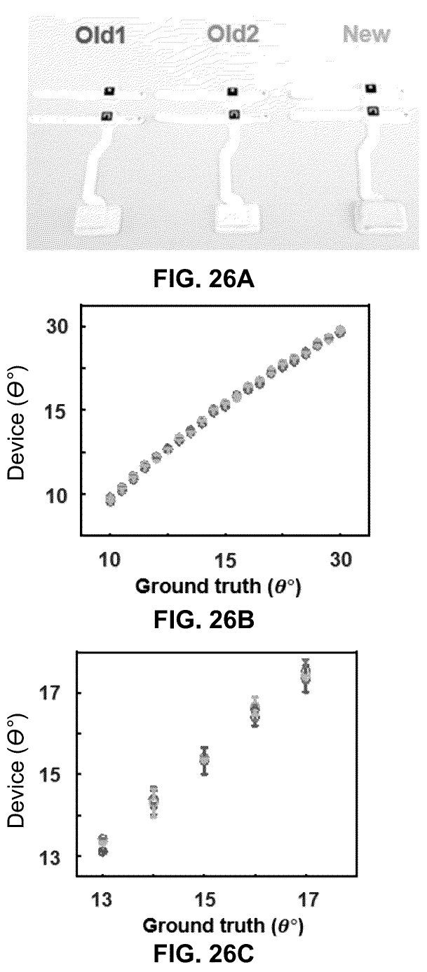

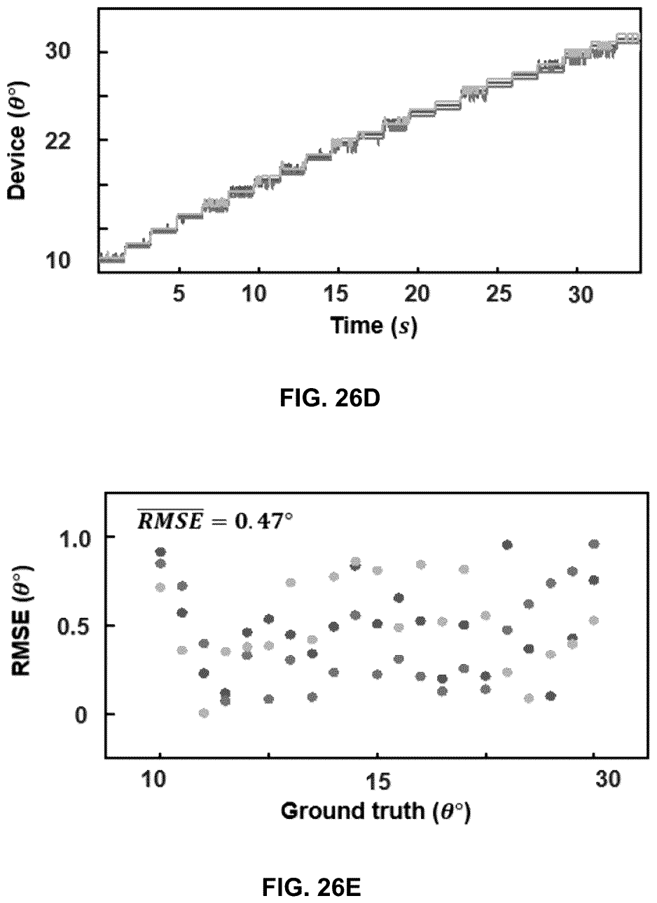

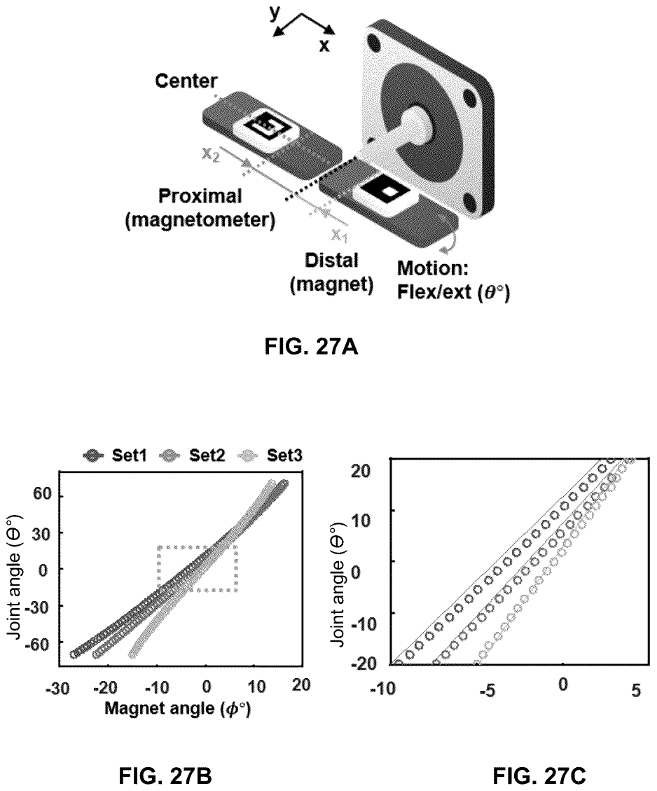

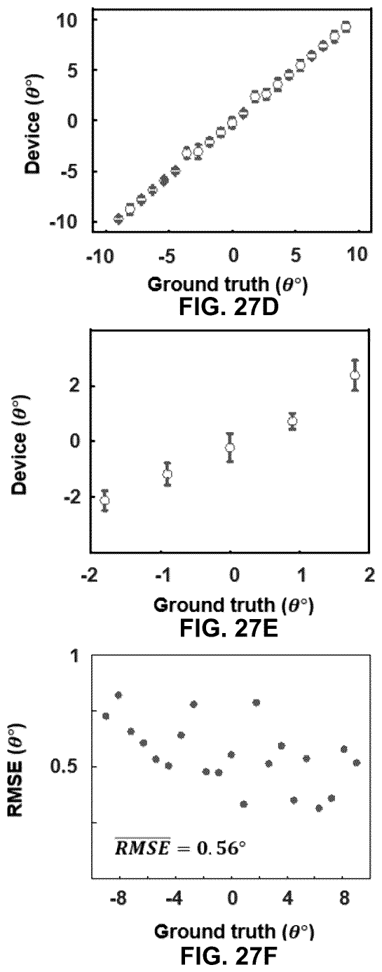

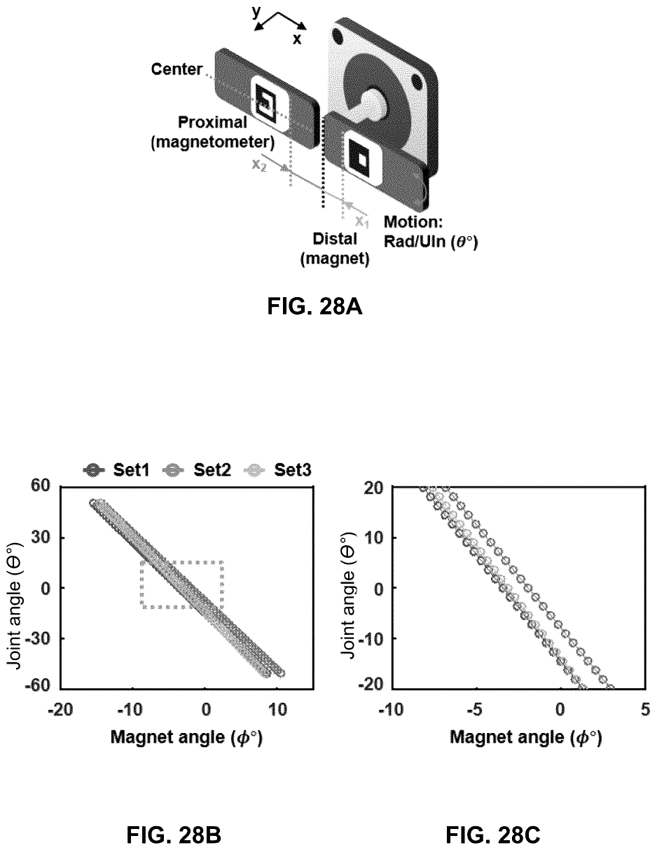

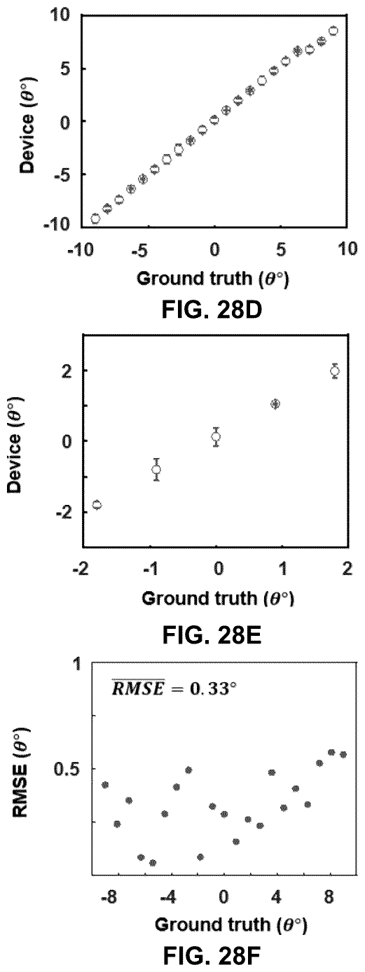

Besides the extrapolation performance and measurement accuracy and precision, the benchtop experimental setup also characterizes key aspects of the FG and WG as sensing systems. The FG exhibits an overall sensitivity of 2.3° joint angle/1° magnet angle across three different distances between the magnet and magnetometer (FIG. 24B, FIG. 24C). Notably, misalignment between the magnet and magnetometer has a negligible effect on device responses (FIG. 25A, FIG. 25B, FIG. 25C). The FG shows highly repeatable responses, both within trials of an individual FG and across FGs of different ages (FIG. 26A, FIG. 26B, FIG. 26C, FIG. 26D, FIG. 26E). Additionally, all three FGs demonstrate resolutions close to 1° degrees. Sensitivities of the WG to angular motions (FIG. 27A, FIG. 27B, FIG. 27C, FIG. 28A, FIG. 28B, FIG. 28C) are 3.8° and −4.2° joint angle/1° magnet angle for the flex/ext and rad/ulnar motions, respectively, across three different distances. The resolution is less than 2° in both motions.

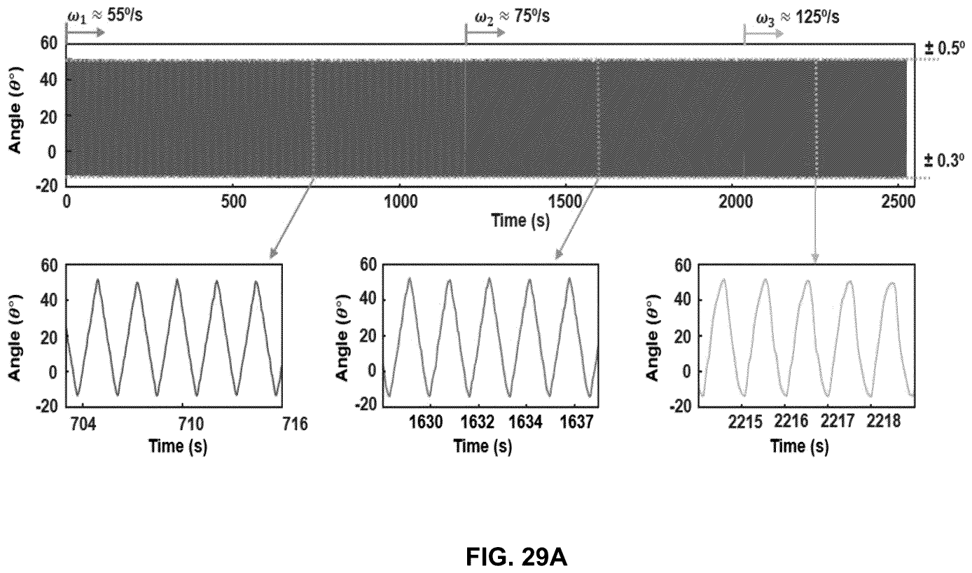

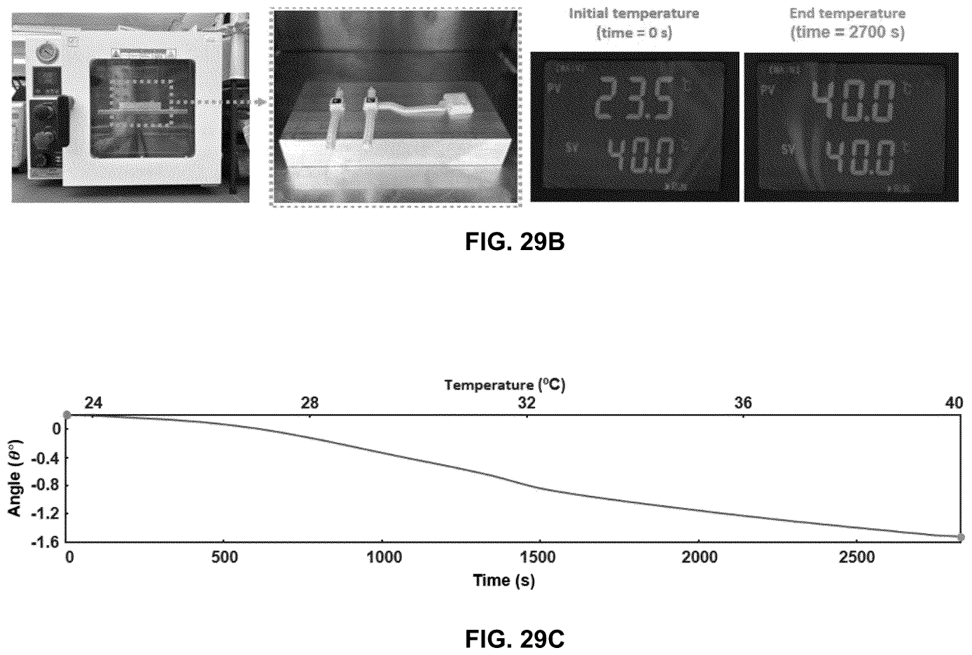

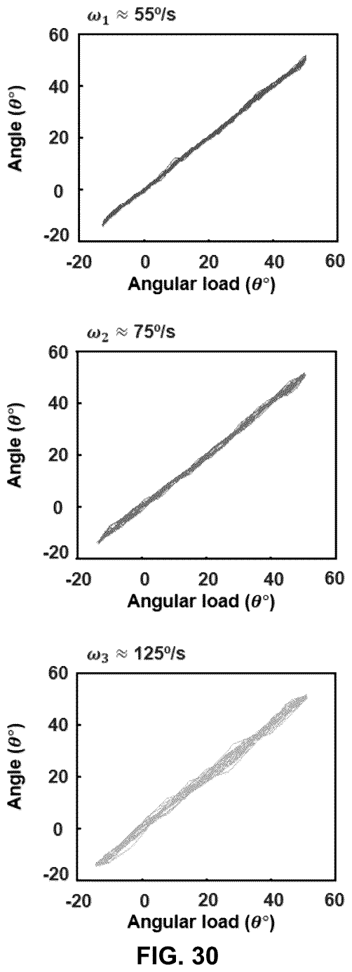

Drift is another important aspect of a sensing system. With negligible drift over 45 min, the FG captures angular motions (−14° to 51°) sequentially applied at three angular speeds (ω₁≈55°/s; ω₂≈75°/s; ω₃=125°/s; FIG. 29A). The FG also demonstrates stable responses under temperature variations (23.5 °C to °C), a common cause of drift in magnetometers, with drift limited to only 1.3% of the full span (FIG. 29B, FIG. 29C). In addition, hysteresis in the FG is negligible (FIG. 30).

### Sensing Hand Digit PIP and IP Joint and Wrist Joint Motions

Targeted joint motions captured by the electrogoniometry systems involve complex 3D movements but can be assumed to consist of a single DOF rotation. Ground truth data for purposes of validation uses 3D marker tracking with improved 3D reconstruction and pose estimation, utilizing Pose 3D and DeepLabCut (DLC). This approach not only provides detailed motion paths but also significantly reduces the ambiguity in movement interpretation by precisely quantifying the angles and trajectories involved.

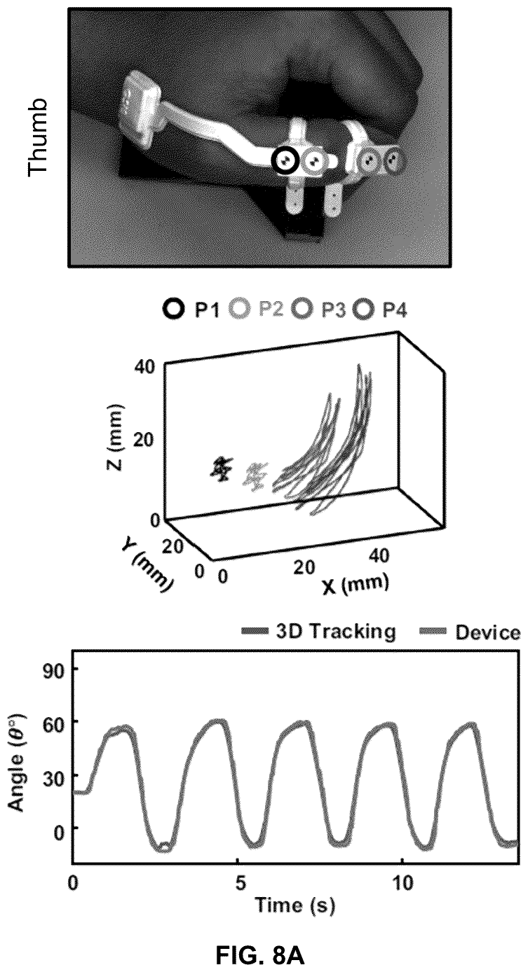

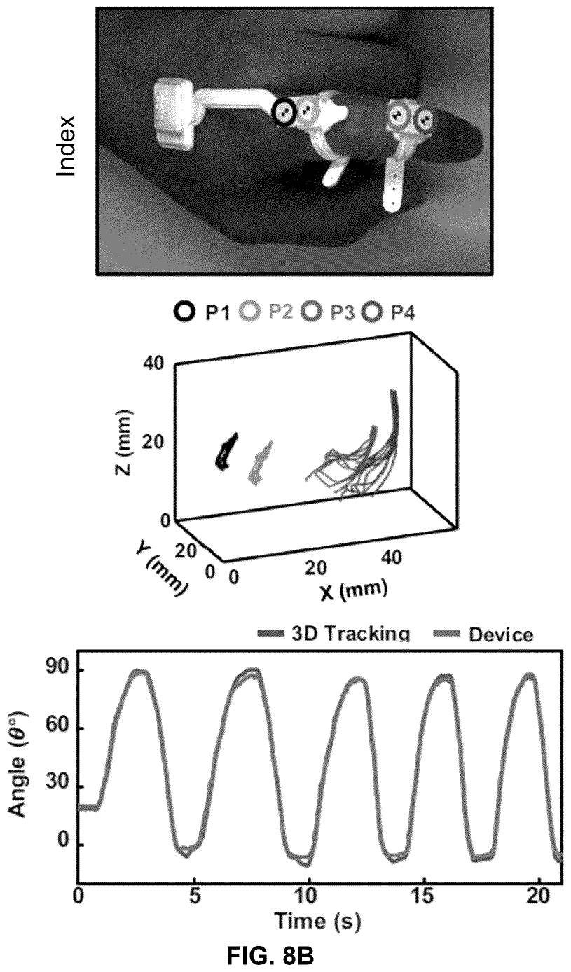

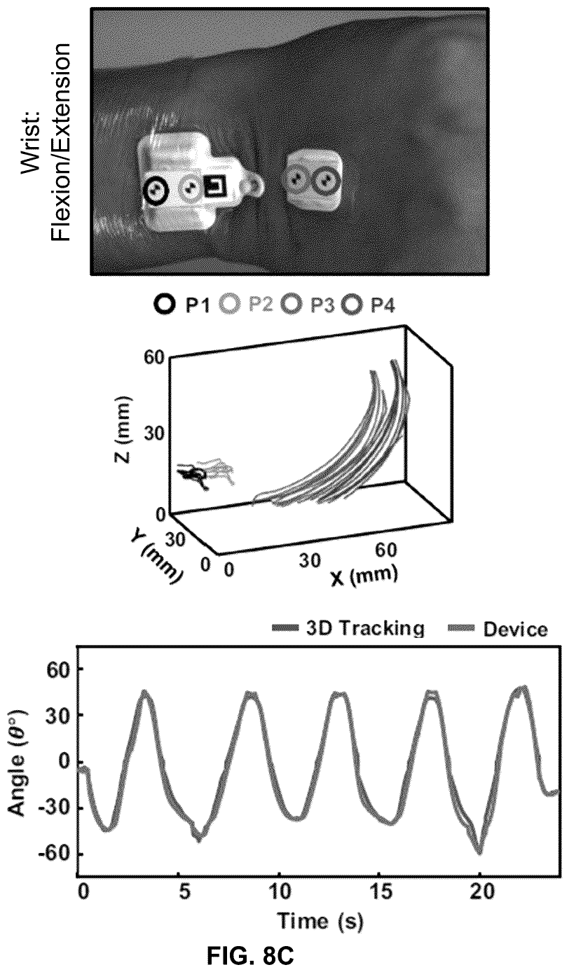

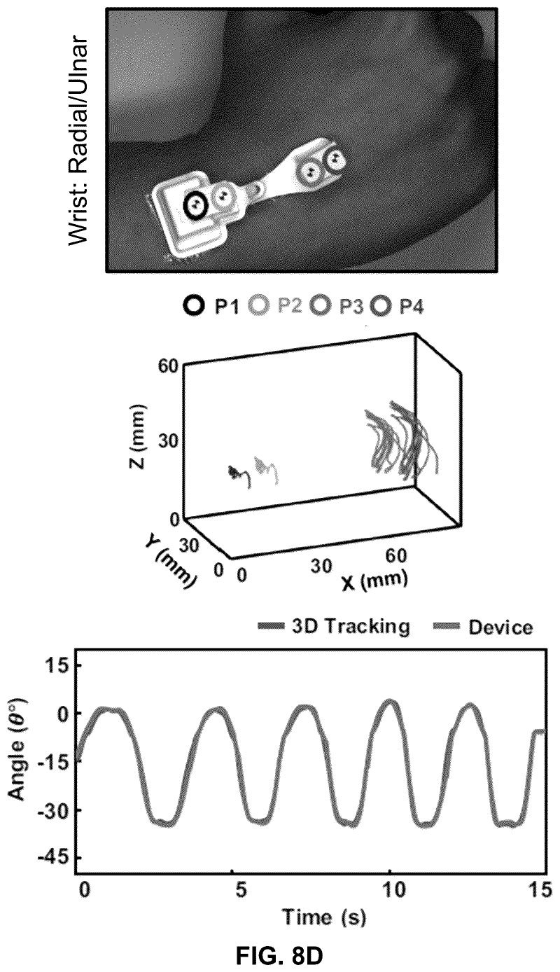

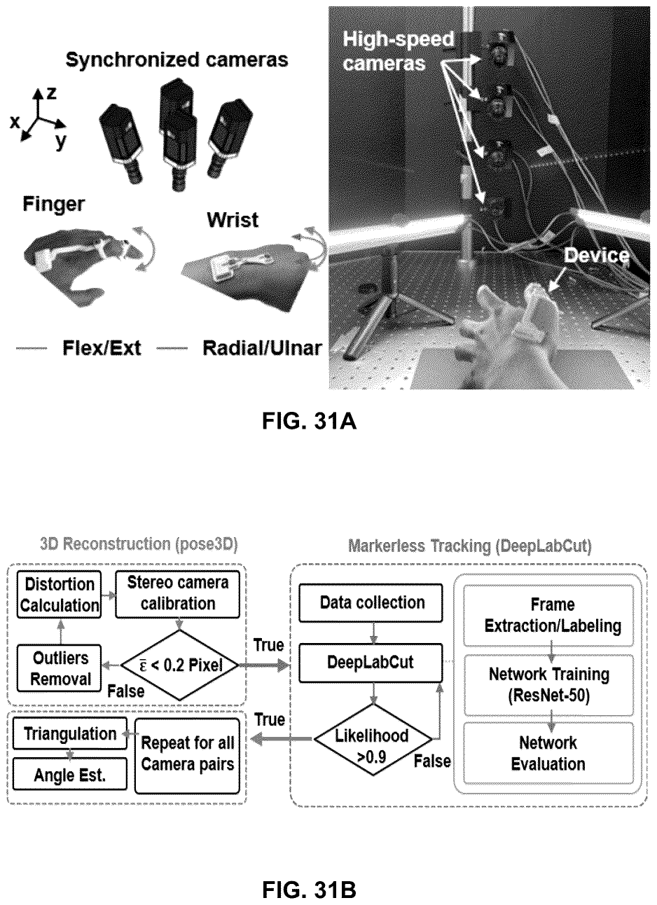

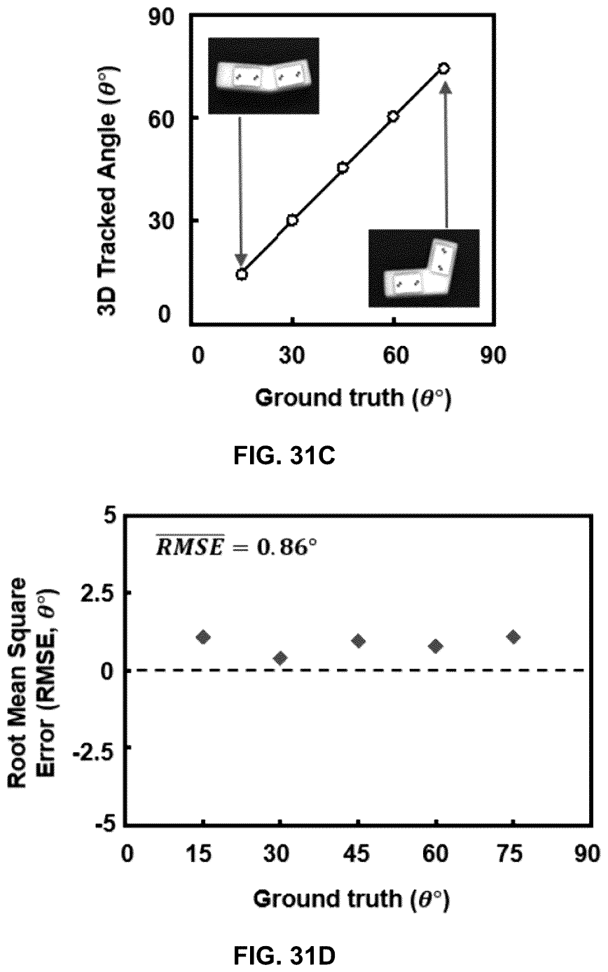

The use of four fiducial markers for this purpose is illustrated in FIG. 8A, FIG. 8B, FIG. 8C, and FIG. 8D. Two markers on each segment represent the proximal and middle (or distal) phalanges of the hand and the carpal and forearm bones of the wrist. The marker tracking process precisely captures and reconstructs the positions of these markers in 3D space from videos recorded by four time-synchronized, high-speed cameras, each operating at 60 fps (FIG. 31A, FIG. 31B). The estimated positions (P1, P2, P3, and P4; FIG. 8A, FIG. 8B, FIG. 8C, FIG. 8D) facilitate the measurement of angles using two vectors: θ=cos⁻¹ ((a×b)/(|a∥b|)), where the vectors, a and b, represent vectors from the positions, P2−P1 and P4−P3, respectively. An overall RMSE of 0.9° within the range of 15° to 75° (FIG. 31C, FIG. 31D) confirms that angles measured in this manner are sufficiently accurate to serve as ground truth.

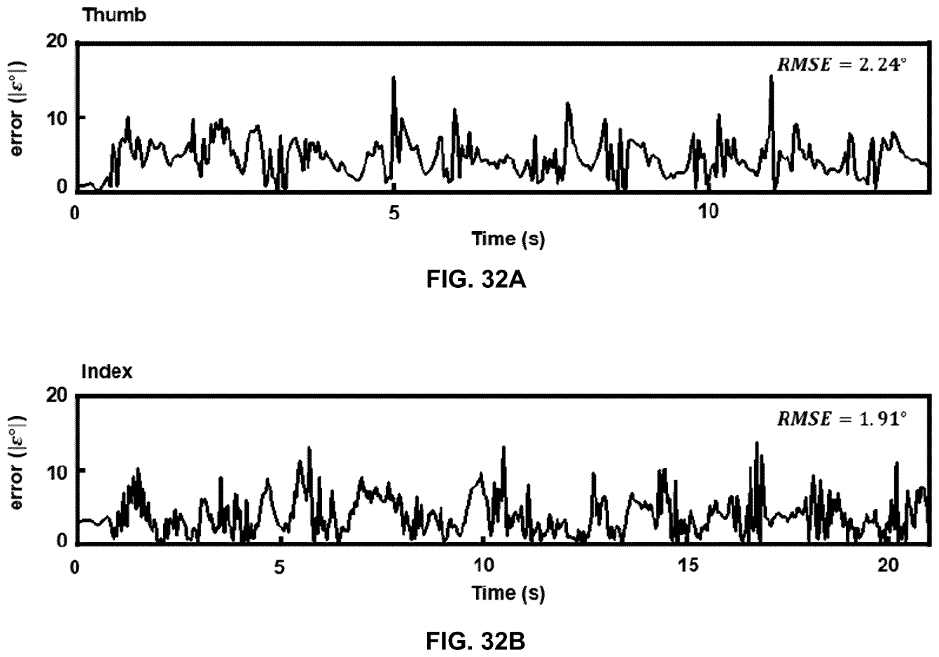

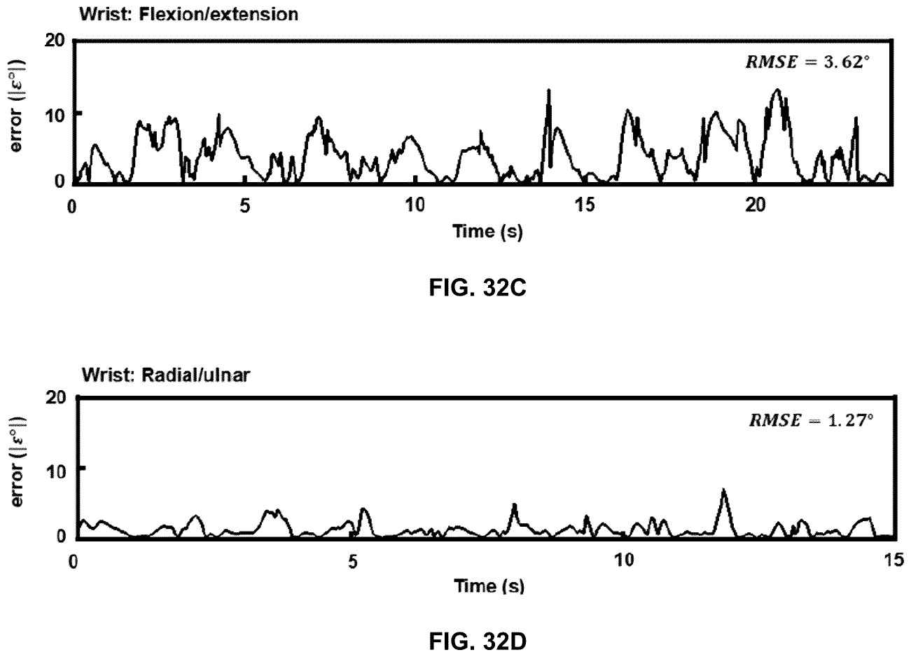

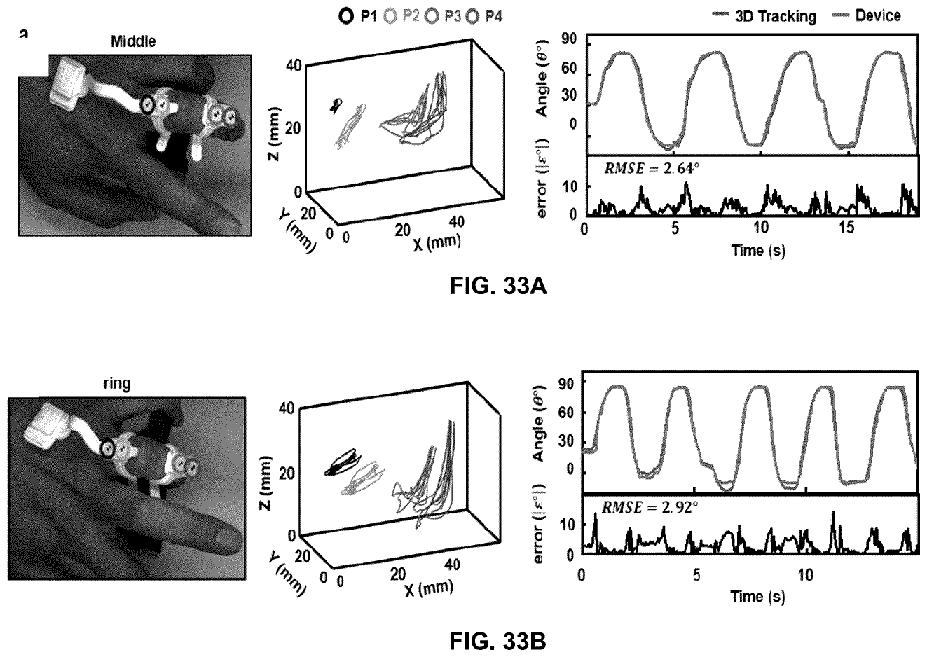

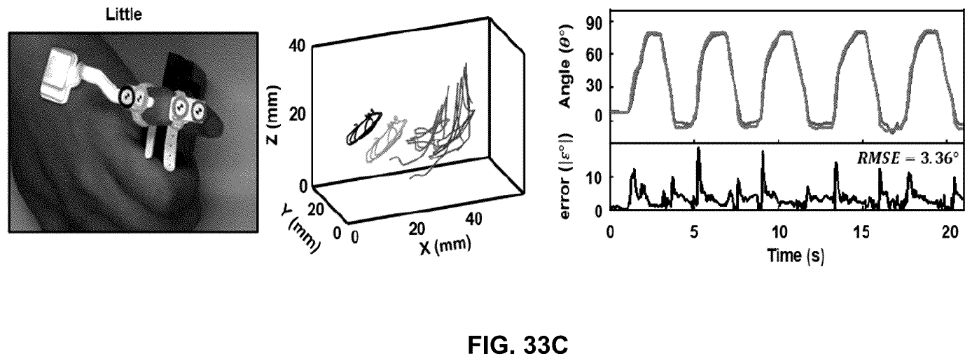

The responses of the FG to spatial motions of the thumb and index finger are demonstrated (FIG. 8A, FIG. 8B). On the thumb, the FG accurately captures motions of the IP joint, ranging from −13.1° (hyperextension) to 60.7° (full flexion), closely aligning with the range of the ground truth, spanning from −11.8° to 59.7°. Despite the limitations in error analysis, an RMSE of 2.2° (FIG. 32A) confirms a high degree of agreement between the ground truth and the responses of the FG. The FG also measures rotational angles of the PIP joint in the index finger, exhibiting its typical range of motion, from −6.9° (hyperextension) to 88.9° (full flexion), with an RMSE of 1.9° (FIG. 32B), indicating strong agreement with the ground truth (from −11° to 90.2°; FIG. 8A, FIG. 8B, FIG. 8C, FIG. 8D). The full flexion angles observed in both hand digits are marginally lower than the documented ranges (Table 3), likely due to interference from the strap bands of the FG. In addition, the tendency of the FG to slightly underestimate hyperextension may result from the mildly degraded performance of the extrapolation under this more practical setup. While the RMSE tends to decrease with increasing independence of finger movement, the little finger shows the highest error (3.4°), likely due to its smaller size that limits optimal placement of the FG despite having relatively high independence compared to the middle, ring, and little fingers (FIG. 33A, FIG. 33B, FIG. 33C). Given the anatomical differences between the thumb and fingers, the experimental results validate that the FG effectively captures flex/ext motions of the PIP and IP joints in hand digits.

The enhanced 3D marker tracking also facilitates a detailed assessment of the performance of the WG in measuring both flex/ext and rad/uln motions of the wrist. For flex/ext motions, the range captured by the WG extends from −57.8° to 48.8°, closely matching the range of the ground truth from −59.6° to 47.7° (FIG. 8C). An RMSE of 3.6° (FIG. 32C) indicates good agreement between these two data sets. The responses of the WG to rad/uln motions with measurements ranging from −35.1° to 3.5°, demonstrates strong agreement with the ground truth (from −35.2° to) 4°, further supported by a lower RMSE of 1.3° (FIG. 8D, FIG. 32D). These improved results likely follow from the reduced range of rad/uln motions compared to flex/ext motions and the use of interpolation in measuring these motions.

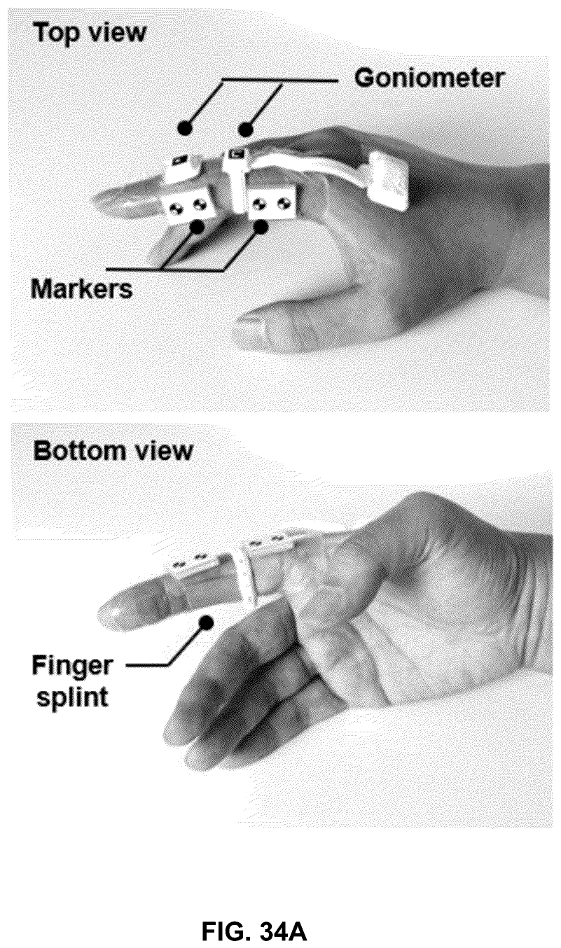

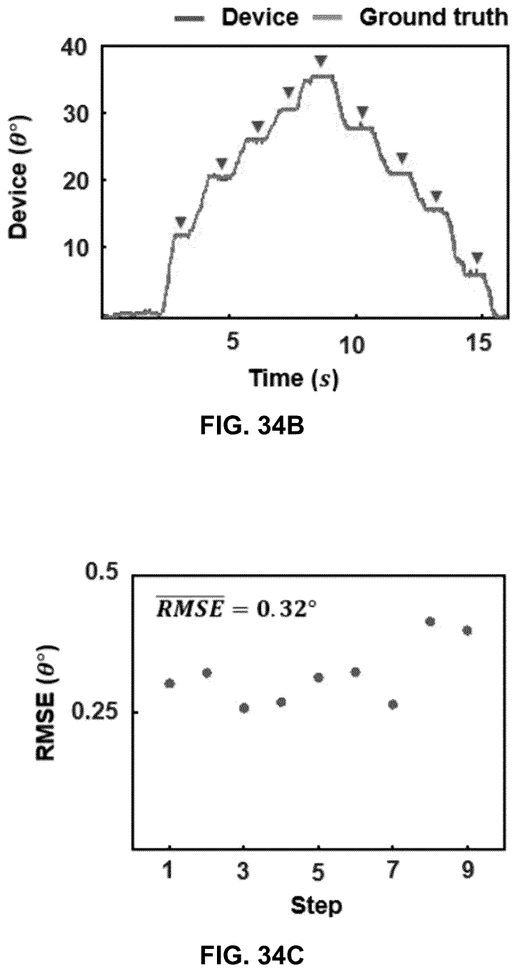

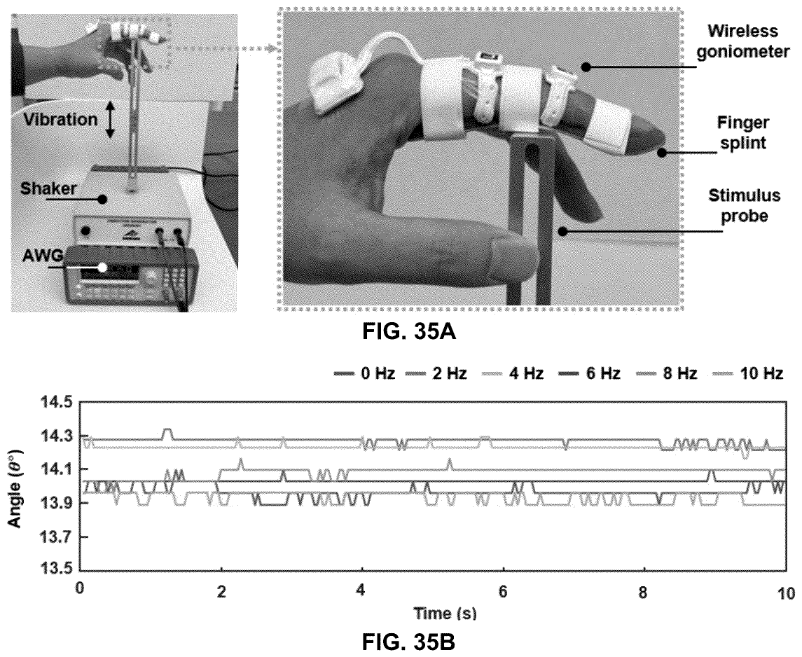

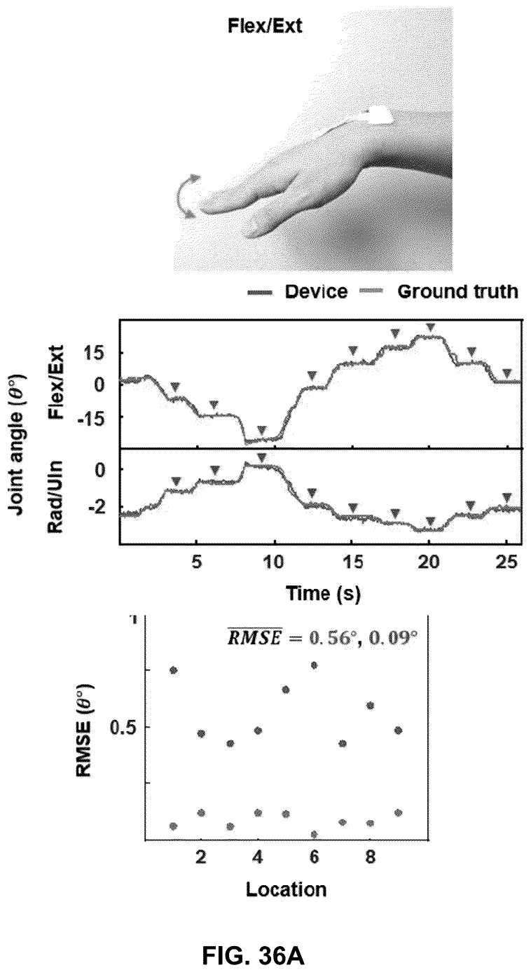

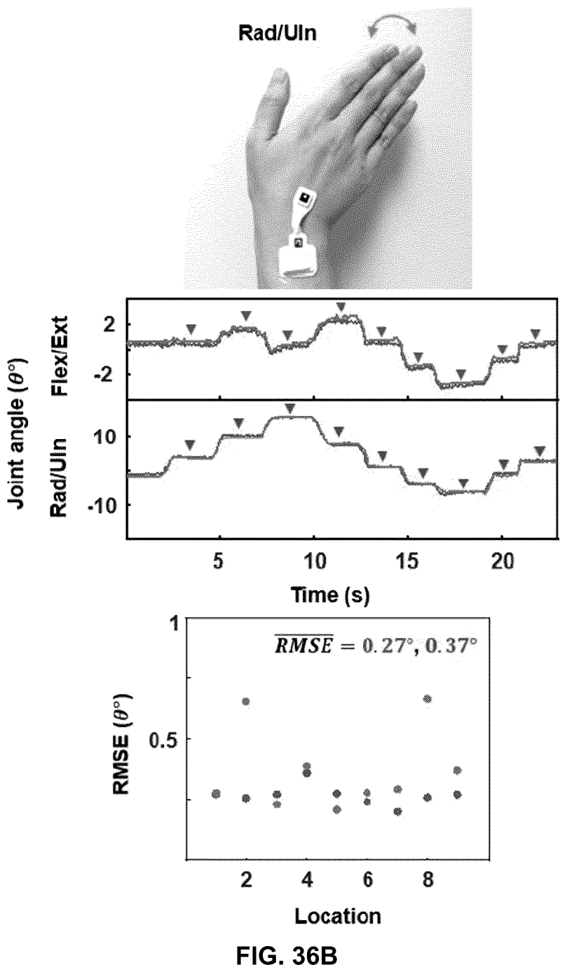

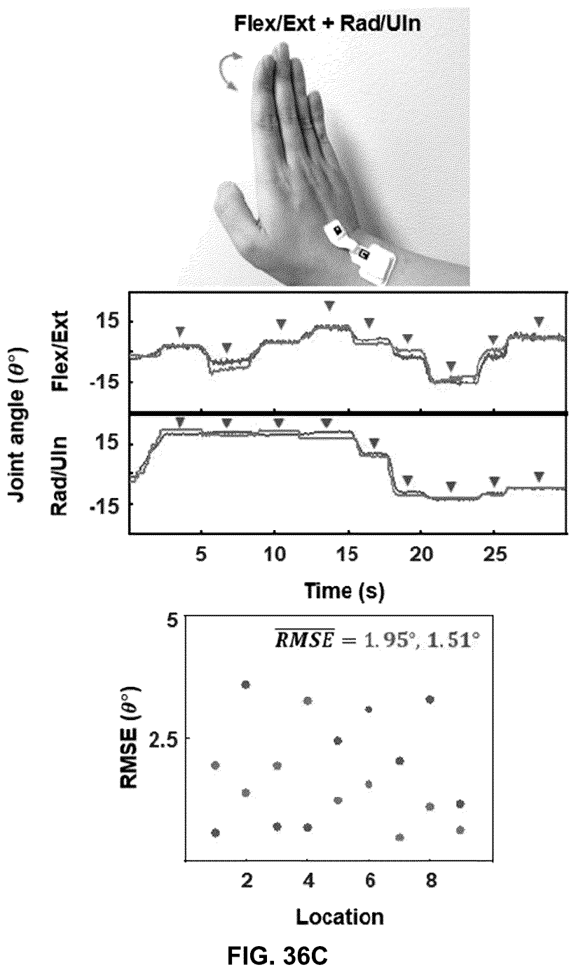

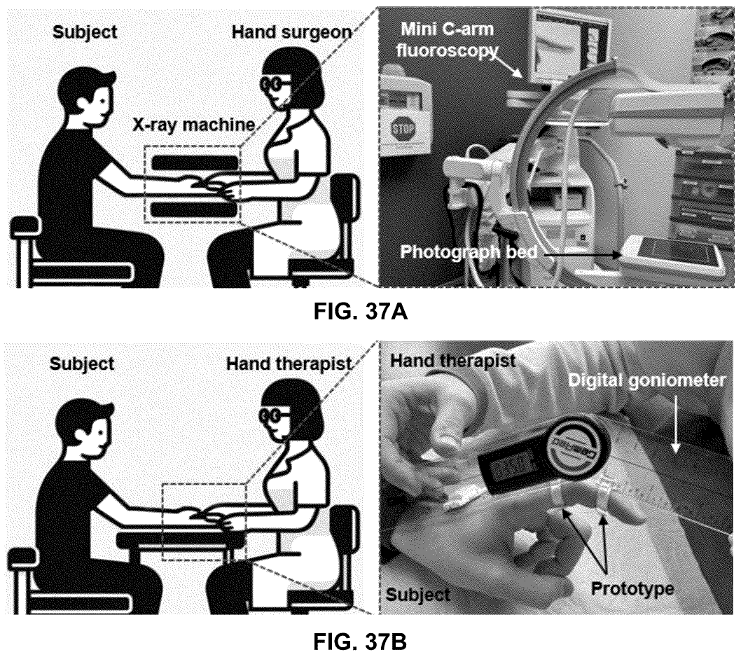

In addition to the experiments on the FG and WG under the free motion condition, both goniometry systems demonstrate their responsiveness to simulated patient conditions, including restricted movements and artificial tremors comparable to human tremors (3 Hz to 12 Hz). The responses of the FG and WG to restricted motion, showing even lower RMSEs (FG: 0.3°; WG (flex/ext): 0.6°; WG (rad/uln): 0.4°) compared to those observed under the free motion condition, likely due to the reduced range of movement (FIG. 34A, FIG. 34B, FIG. 34C, FIG. 36A, FIG. 36B). Furthermore, the FG exhibits stable responses under artificial tremor (FIG. 35A, FIG. 35B), suggesting that the goniometry systems are capable of reliably monitoring angular motions of patients with high accuracy and precision.

### Sensing Finger PIP Joint Motions During Simulated Supervised Hand Therapy

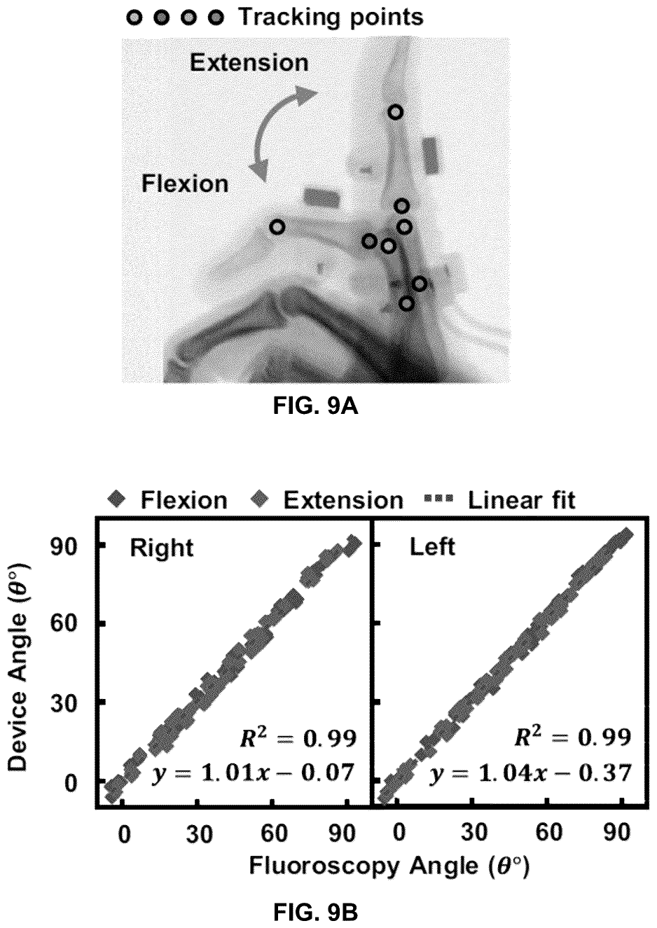

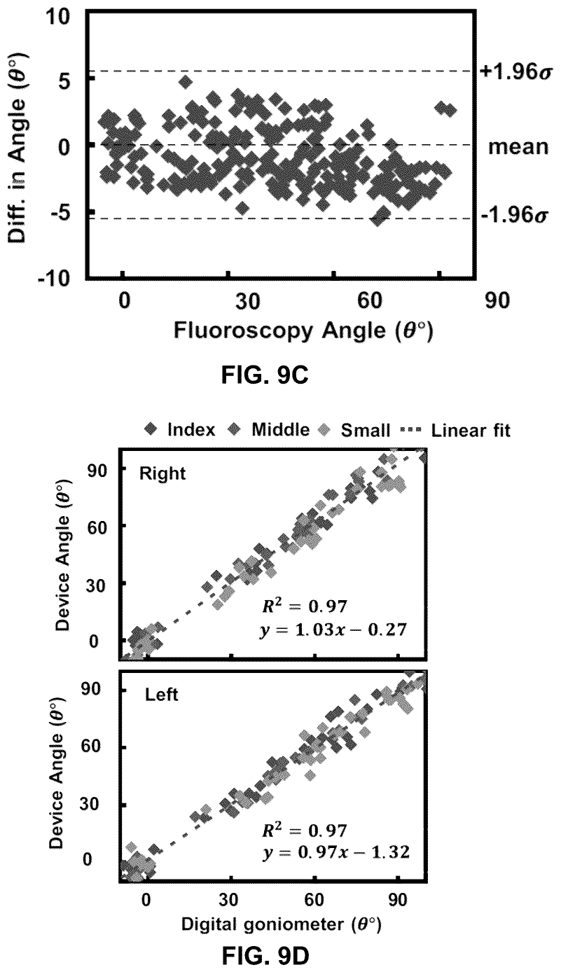

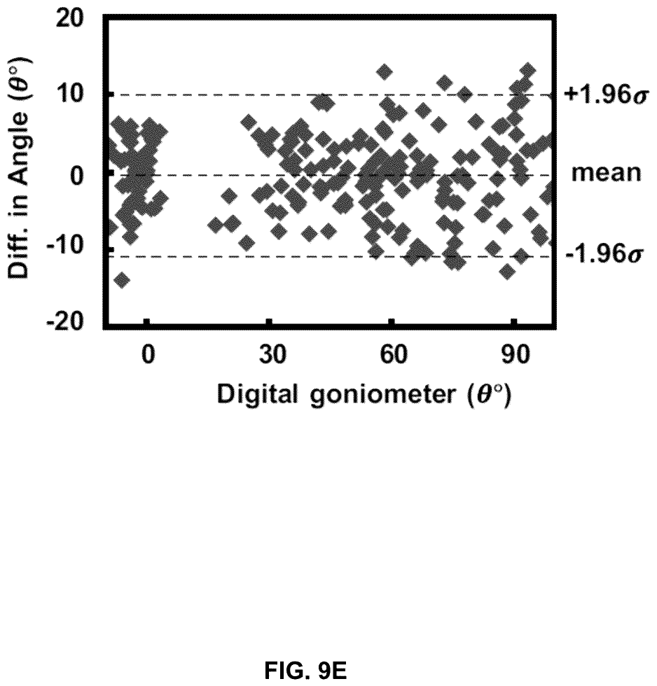

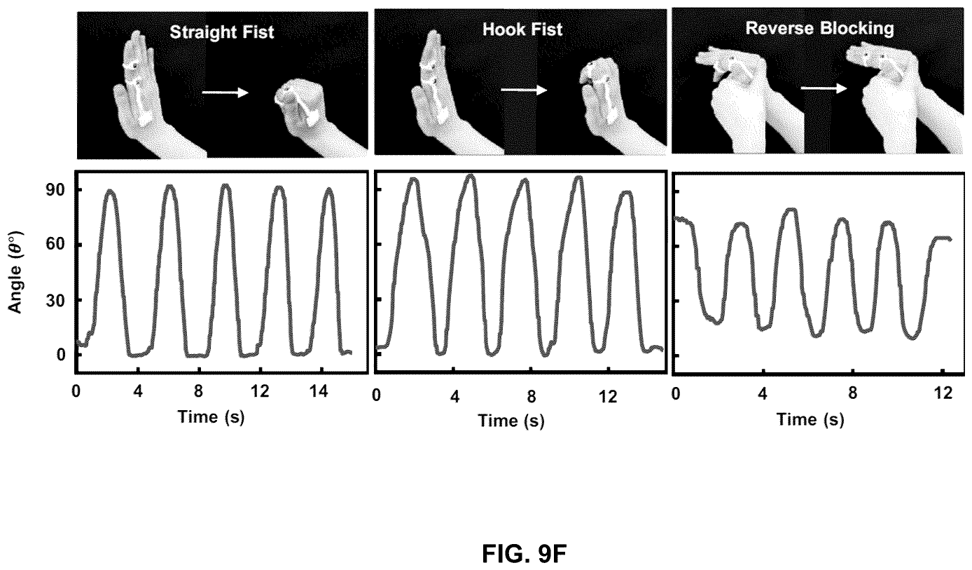

As verified by the full 3D marker tracking, the FG can effectively monitor PIP and IP joint motions under practical settings, with high precision. Demonstrating the efficacy of the FG in clinical and therapeutic contexts, however, is a fundamental step in assessing its usability and reliability as a biomedical wearable. This technical validation includes exploring three data collection protocols with healthy volunteers as subjects in physical therapy settings. The first protocol focuses on comparing PIP joint angles in index fingers measured by the FG to those from clinical ground truth. Data for this comparison came from five healthy volunteers (Table 5) wearing the device on either their left or right index finger while executing flex/ext motions under the supervision of a hand surgeon (FIG. 37A). Markerless tracking was performed on videos recorded by a mini C-arm fluoroscopy (Fluoroscan InSight FD) at 30 fps during the experiments (FIG. 9A). Similar to the full 3D marker tracking, DLC facilitates angle estimations using vectors obtained from this 2D markerless tracking by recognizing and tracking four points in 2D space—the head and base of the middle phalanx and the head and center of the proximal phalanx. Linear fitting functions with slopes close to 1, along with Pearson correlation coefficient (r value) exceeding 0.99, confirm strong agreement between angles from these two measurements (FIG. 9B). This finding is further corroborated in graphs that show deviations of less than +5° between these two measurements within a 95% confidence interval (FIG. 9C). Importantly, this range of accuracy, within an error margin of 5°, is considered acceptable for goniometric measurement of finger joints.

TABLE 5
Hand size of healthy volunteers who
participated in human trials.

| Sub- | L-ML | L-MC | R-ML | R-MC | |
|---|---|---|---|---|---|
| ject | (cm) | (cm) | (cm) | (cm) | Study |
| 1 | 18.5 | 6 | 18.5 | 6 | FL |
| 2 | 18.5 | 6.5 | 18.5 | 6.5 | FL |
| 3 | 18.0 | 5.9 | 18.0 | 61 | FL |
| 4 | 18.7 | 6.3 | 18.5 | 6.6 | OT; FL |
| 5 | 18.2 | 5.9 | 17.9 | 5.9 | OT; FL |
| 6 | 18.6 | 5.3 | 18.2 | 5.3 | OT |
| 7 | 19.0 | 7.1 | 18.7 | 7.0 | OT |
| 8 | 16.9 | 5.9 | 17.0 | 6.1 | OT |
| 9 | 18.2 | 5.9 | 17.9 | 5.9 | OT |
| 11 | 17.2 | 5.5 | 17.1 | 5.6 | OT |
| 12 | 19.4 | 6.0 | 19.3 | 6.2 | OT |
| 13 | 17.5 | 5.6 | 16.9 | 5.9 | OT |
| 14 | 17.1 | 6.2 | 17.3 | 6.3 | OT |
| 15 | 20.5 | 6.5 | 20.1 | 6.5 | OW; LT; TB |
| 16 | 18.5 | 5.7 | 18.2 | 5.8 | OW; TB |
| 17 | 19.5 | 6.2 | 19.7 | 6.4 | OW; TB; PP |

Abbreviations: fluoroscopy (FL), an occupational therapist (OT), or activities, including office work (OW), long term measurement (LT), throwing a ball (TB), and playing a piano (PP).
The hand size is defined by the length from the wrist crease to the tip of the middle finger (ML) and the circumference of the middle finger (MC).
The letters with hyphens, L- and R-, denote the left hand and right hand, respectively.

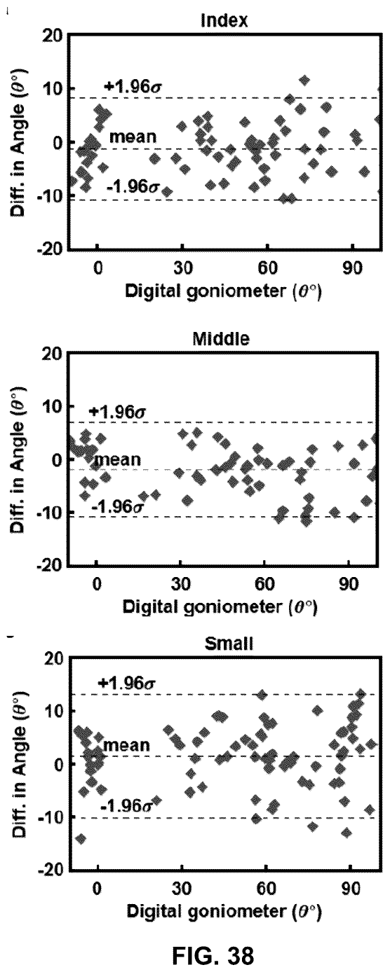

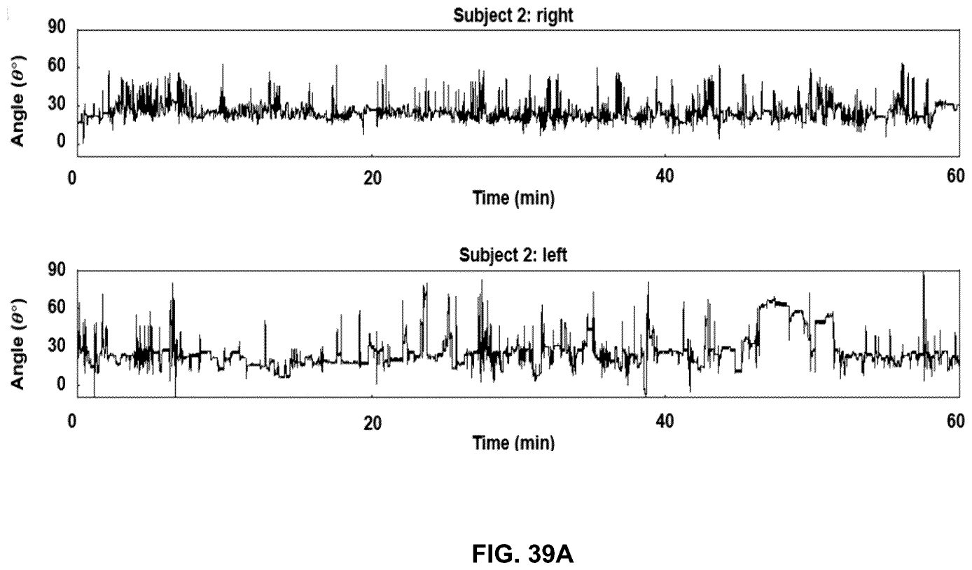

The general practice in hand therapy sessions involves assessing the range of motion of a joint using a handheld goniometer. The second protocol, therefore, aims to compare the responses of the FG to this general practice to determine the clinical relevance of the device. The PIP joint angles of the index, middle, and small fingers on both hands of ten healthy volunteers was measured by an occupational therapist using a digital goniometer (82311-200 P, GemRed; ±0.3° accuracy; FIG. 37B). The data shown in FIG. 9D arise from the same rater, to eliminate inter-rater variations. Details on the session and volunteers are in FIG. 37B and Table 5. As with the results of the first study, slopes close to 1 in linear fitting functions and r values of 0.98 demonstrate strong accordance of the FG with the general practice. These two methods, however, show almost twice the deviation (−10.9° to 9.9° within a 95% confidence interval) compared to the first study (FIG. 9E). Along with the estimated errors in the FG from the first study, human errors may contribute. Notably, increased errors for small fingers (FIG. 38), reflect human errors, typically related to the design of the digital goniometer, which is unfavorable for small fingers (FIG. 37B). Nonetheless, this level of error remains within a reasonable range, considering the combined error from defects in the FG (5°) and human error (8°) totals 9.4°.

The third protocol demonstrates the compatibility of the FG with active ROM exercises that patients routinely perform during supervised hand therapy sessions and the HEP. The FG can provide patients with continuous feedback during such exercises, which is not possible with conventional measurement techniques. Use of the FG in hand therapy sessions could thus offer significant benefits in a therapeutic context. The responses of the FG during three specific active ROM gliding exercises: straight fist, hook fist, and reverse blocking are shown (FIG. 9F). The device provides continuous feedback to a healthy volunteer (Table 5) while conducting these exercises, demonstrating compatibility with standard rehabilitation practices. The data show similar ranges of motion for the straight first (−1.1° to) 92.5° and hook first (−0.8° to) 97.8°, while the reverse block shows a distinct range of motion (9.6° to) 80° from those two (FIG. 9F). These ranges remain within the reported range of motion for the finger PIP joints (Table 3). This unique function in offering real-time angle feedback to users could significantly enhance therapeutic outcomes, as previous studies show that such feedback can motivate users and help them track progress. This real-time feedback, particularly in variations noted in exercises like the reverse block, suggests that applications of the FG could help to fine-tune therapy protocols to maximize patient recovery based on immediate biomechanical responses.

### Sensing Finger PIP Joint Motions During Activities

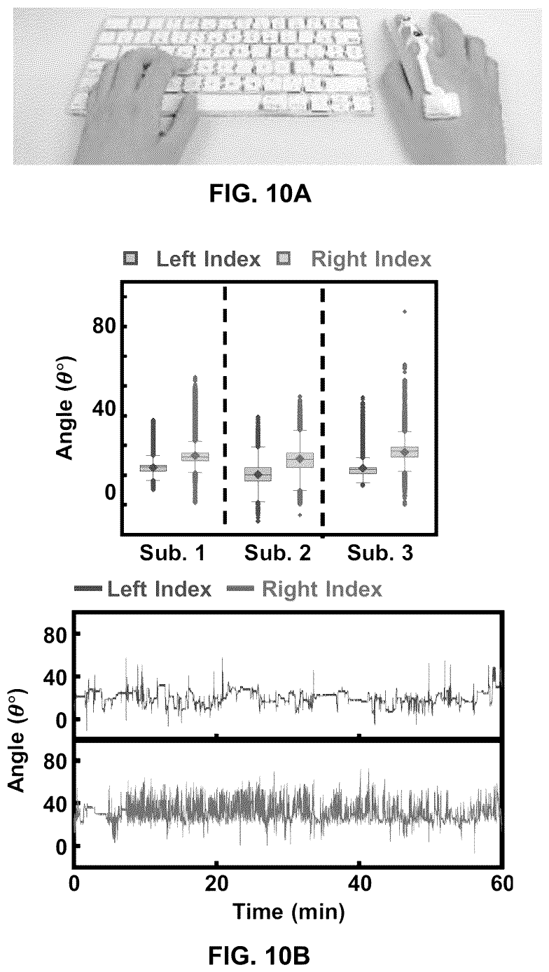

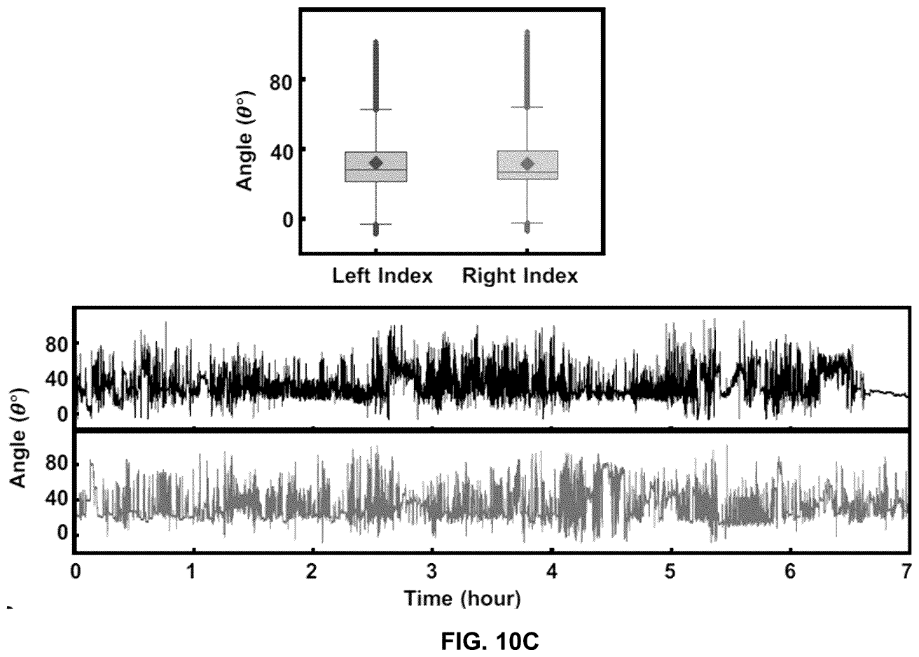

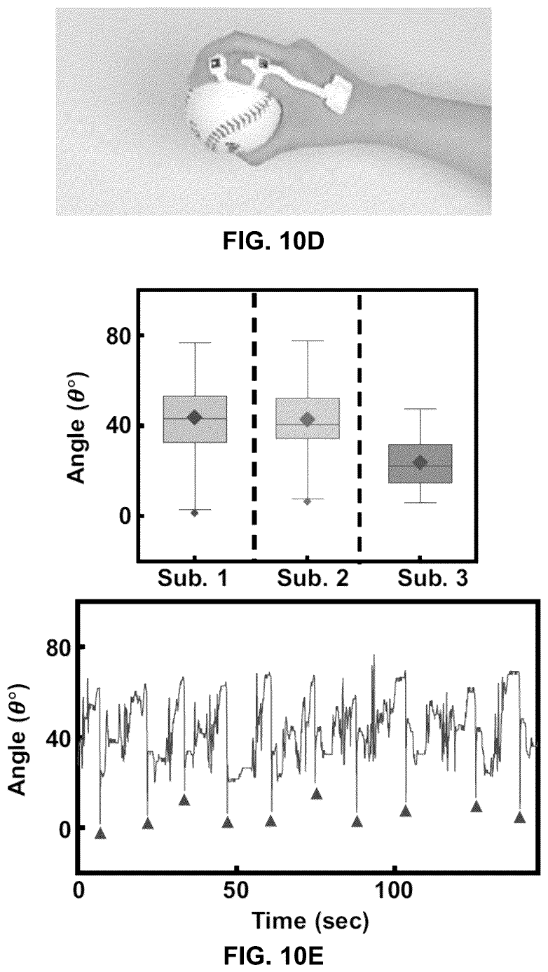

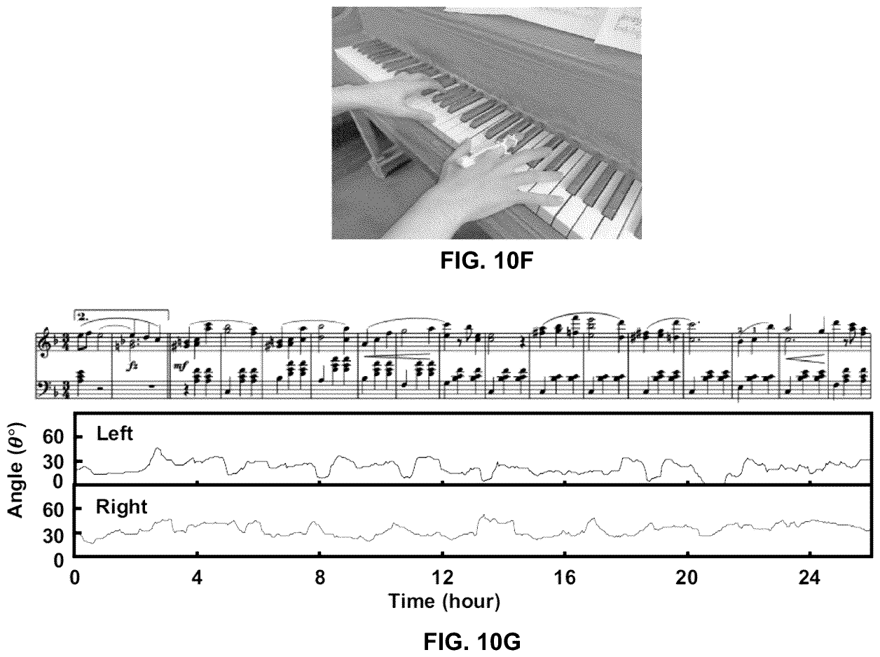

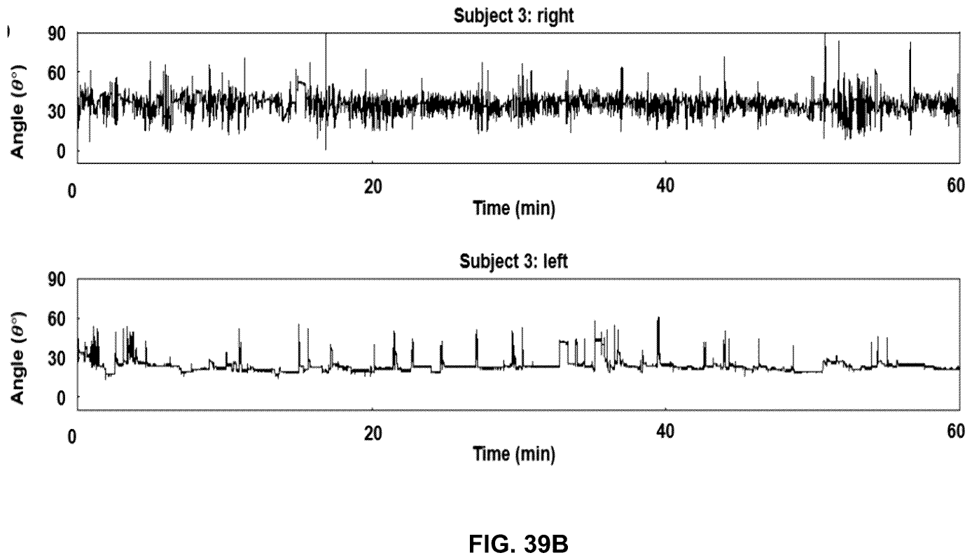

Monitoring the kinematics of a targeted finger joint during occupational activities adds another important layer from clinical and therapeutic perspectives. The feedback provided by the FG could be particularly beneficial for both clinicians and patients, helping to accelerate their rehabilitation progress. An office setup with a keyboard and mouse (FIG. 10A), may represent a typical environment for many individuals. Data on the FG from three healthy volunteers (Table 5) who participated in this study show different ranges of motion but similar trends (FIG. 10B). Although the range of motion for PIP joint in the index fingers varies among individuals as well as between their hands (68.3±15.3°), the average angles across all PIP joints are comparable (28.2)±5.8°. Notably, the left index finger exhibits lower ranges of motion than the right index finger. This variation likely stems from the difference in primary tasks of the right and left index fingers-keyboard typing and mouse clicking/scrolling, respectively. The time-series data (FIG. 10B, FIG. 39A, FIG. 39B) clearly illustrates these trends. Long-term measurements capture PIP joint in the index fingers of a healthy volunteer (subject 15) in a workplace setting. Compared to results from the office setup, these long-term measurements reveal a slightly higher overall angle (31.9±) 0.4° and a wider range (112.5)±2.9°, reflecting more dynamic motions from various activities. In situations where a rehabilitation or injury-prevention plan requires avoidance of extreme flexion or extension, this device could prove useful by notifying the user of motion beyond a specified range. Ideally, this process of biofeedback could train the user to sense when they approach the bounds of the specified range, thus allowing the device to be discontinued after sufficient training (while still having ongoing benefit).

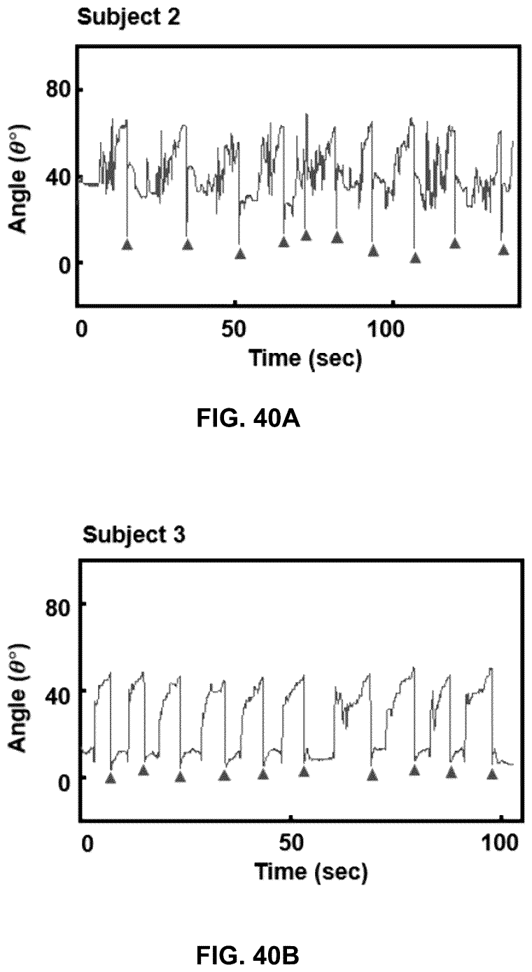

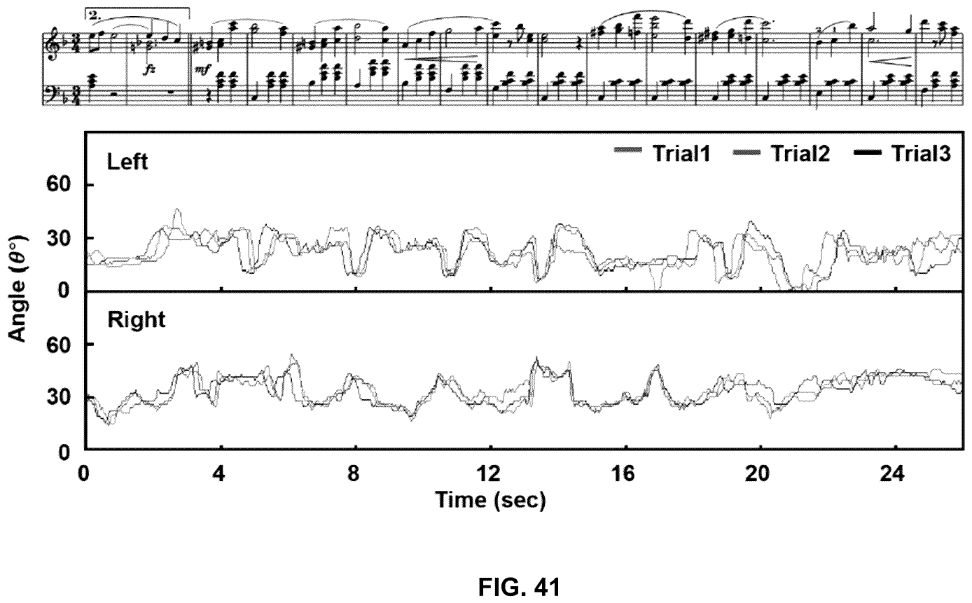

Beyond clinical applications, another advantage of using this system is its ability to track joint kinematics during vigorous activities, including sports and playing musical instruments. This capability is demonstrated by the FG during a baseball throw (FIG. 10D). Results from three healthy volunteers (Table 5; FIG. 10E) reveal varying ranges of motion in the PIP joint of the right index finger (62.5+) 18.5°, reflecting differences in the throwing habits of each volunteer. In addition, the time-series data (FIG. 10E, FIG. 40A, FIG. 40B) illustrate angles while holding and releasing a ball. Such results may be useful for analyzing pitching techniques. The usability of the FG extends to tracking PIP joint motions while playing musical instruments (FIG. 10F). The changes in angle of the PIP joints in the left and right index fingers are shown while a user plays a section of Kaiser-Walzer, Op. 437 (FIG. 10G). The time-series data show that the FG effectively captures the joint motions of both fingers during the performance. Notably, the measured angles exhibit slight variations between trials, even though the same musician plays the same section of the song (FIG. 41). Through advanced data analysis, such data can assist musicians in refining their techniques or adjusting their playing habits by enabling comparisons of motion data across different performances.

### Discussion

The electrogoniometry systems presented here facilitate continuous monitoring of joint kinematics while addressing typical challenges associated with wearable electrogoniometers, which would benefit both patients and clinicians. The operation relies on simple measurements of the magnetic field strength from a permanent magnet, with the magnet and magnetometer placed distal and proximal (respectively) to a target joint. Computer vision incorporated in a smart device precisely calibrates responses of the magnetometer to joint angles. This instant and easy calibration strategy, designed to take 4 seconds, can bypass the need for additional equipment, large training datasets for machine learning, and significant computational power for magnet pose reconstruction. Consequently, patients could utilize the presented systems with real-time biofeedback displayed on an intuitive GUI in a routine and frequent manner, regardless of the setting or the presence of a clinician.

Given the results from comprehensive experiments including physical simulations, demonstrations with two ground truth methods, and human trials, the technology—demonstrating clinically acceptable reliability—would reduce previously reported uncertainties resulting from goniometers and practitioners. The strong compatibility with general rehabilitation exercises shown by the FG would allow both patients and clinicians to receive real-time joint angle biofeedback during exercise. Ideally, this technology could be made available to patients who require routine performance of a HEP. They would don the device at home for each exercise session, and receive real-time feedback, rather than (approximately) weekly feedback only when visiting a therapist. This routine biofeedback may facilitate better engagement with the exercise program, help the patient push harder to gain improvement, and ultimately facilitate the achievement of a superior outcome. Future study will specifically examine how this device truly impacts the patient experience and clinical outcomes.

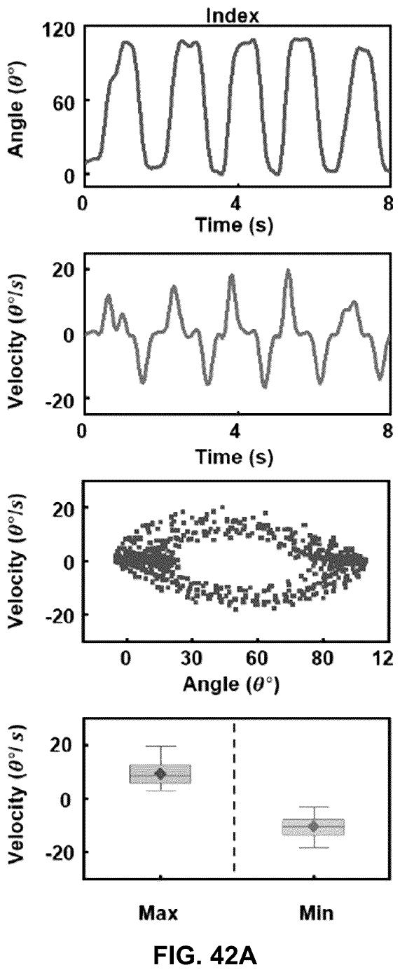

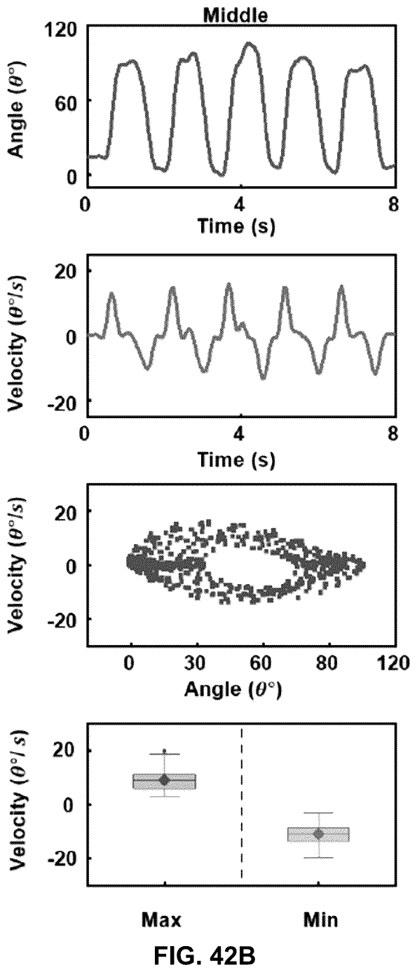

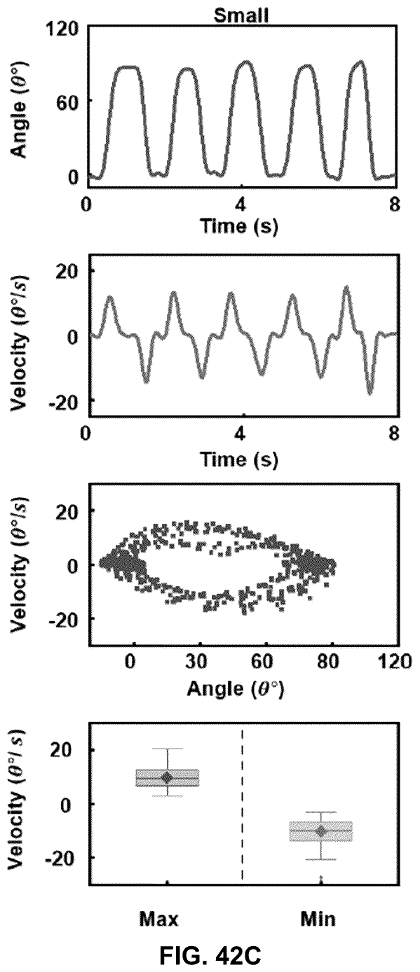

Additionally, the ability of the system to perform long-term continuous monitoring during daily activities would be advantageous for tracking joint usage throughout the day, particularly benefiting patients who require some feedback to avoid the extremes of motion (i.e. patients in the early phases of flexor tendon rehabilitation). Furthermore, additional kinematic parameters, including angular velocity and acceleration (FIG. 42A, FIG. 42B, FIG. 42C), may serve as useful biomarkers for evaluating rehabilitation progress. Previous work has used such parameters to monitor hand rehabilitation in post-stroke patients and confirm relationships between angular speed and muscle weakness in Parkinson's disease. By leveraging the full rotation matrix of ArUco markers and the orientation of a magnet, multi-DOF monitoring of a wrist joint (FIG. 36A, FIG. 36B, FIG. 36C) suggests a promising prospect for the future generalization of this technology to larger and more complex joints. While the primary target of the electrogoniometry systems is medical diagnostics and rehabilitation, the full kinematic parameters could also be applied to physical activities, as demonstrated in this work with joint angles. Further opportunities may emerge in both medical and physical activity contexts when the systems are integrated with immersive technologies.

### Materials and Methods

### Device Preparation

A commercial electronic design software package (Eagle 9.6.2, Autodesk) was used to design the fPCB. Custom firmware was developed using an integrated development environment (Embedded Studio, SEGGER) and uploaded to the BLE SoC. To mitigate potential electromagnetic interference from nearby metallic components, a wireless charging coil was integrated with an NFC film (IBQ15, TDK Corporation) and then tuned using a set of ceramic capacitors. This electronics preparation was completed by folding the wireless coil onto the fully populated Fpcb (FIG. 11A, FIG. 11B).

Silicone enclosures for encapsulating the electronics with a 3.7-V lithium polymer battery (45 mAh; dimensions: 15 mm length, 12 mm width, and 4 mm thickness) were prepared using a set of acrylic molds manufactured with a benchtop milling machine (Roland MDX540). Liquid silicone (Silbione RTV 4420) dyed with white pigment (Silc Pig, White, Smooth-On; 10 wt. %) was poured into the molds and then cured in an oven at 80 °C for one hour. The top shells of each segment contained slots to mate with black patterns of ArUco markers. After cooling, the electronics and the battery were sandwiched between silicone shells in the molds, which were then filled with liquid silicone (Eco-Flex 0035, Smooth-On). The assembly was clamped and left at room temperature for 5 min, allowing the liquid silicone to solidify and bond the two silicone shells together. Excess material was trimmed using a custom cutting die to achieve the desired device shape. For the strap of the FG, seven holes were punched at 5 mm intervals using a 1.5 mm biopsy punch, and a custom pin—an assembly of a 3D-printed stud and a copper hollow rivet—was inserted and secured with silicone adhesive (Sil-poxy, Smooth-On; FIG. 11A). For the WG, a single hole was made on each segment, and a larger custom pin was attached to the magnet segment using the same adhesive (FIG. 11B).

The black patterns of ArUco markers were fabricated separately by casting the Silbione RTV in a machined acrylic mold. Similar to the white silicone shells, 10 wt. % of black pigment (Silc Pig, Black, Smooth-On) was used to dye the silicone to achieve high contrast for ArUco markers. The black-dyed silicone was cured in an oven 80 °C for one hour, and then peeled off from the mold. The black patterns were refined by cutting excess material with a razor blade and secured in the slots of the white shells using Sil-poxy.

### Experimental Setup

A custom motorized stage, constructed on an aluminum breadboard, possessed a stepper motor (1478, Pololu), a commercial motor controller (Tic T825, Pololu), a 3D printed rod with acrylic plates, and manual stages (FIG. 12A, FIG. 12B, FIG. 12C, FIG. 12D, FIG. 12E, FIG. 12F). This motorized stage was designed to characterize responses of the electrogoniometry systems under constrained conditions, focusing on their accuracy and reliability. The main component, the stepper motor, operated smoothly at targeted rates through 1/16 microstepping, managed by the Teensy 3.6 (PJRC) which communicated with the motor controller via I²C using custom Arduino code. A 3D printed rod (125 mm length), connected to the motor rod, effectively positioned the magnetometer at a sufficient distance from the motor to negate any fringing magnetic fields (FIG. 12B). Two acrylic plates, designed to simulate the structure of targeted joints, were mounted—one at the rod's tip (distal) and the other fixed on an aluminum bracket (proximal)—and aligned precisely with manual stages. Adjusting the configuration of the 3D-printed rod and acrylic plates enabled the simulation of flex/ext and rad/uln motions (FIG. 12C, FIG. 12E).

Stepper motors are typically considered highly reliable, leading to often being used without encoders. Therefore, the motions of the custom motorized stage were recorded at 60 fps using a smart device (iphone XR, Apple Inc.). These videos were subsequently analyzed with an open-source video analysis package (Tracker, Open Source Physics). Three fiducial markers-located on the distal, proximal, and at the center of the joint-facilitated the estimation of joint angles at each video frame (FIG. 12D, FIG. 12F).

### Collecting Reference Functions for Extrapolation

The same benchtop experimental setup was employed to collect reference functions for extrapolation during the calibration step. Magnetic field strengths read by a magnetometer typically depend on the relative distances between a magnet and a magnetometer. To explore this dependency, responses of the magnetometer were investigated by adjusting these distances (FIG. 19A). Both the magnet cuboid and the magnetometer were moved in 1 mm longitudinal steps within the distances investigated ranging from 5 mm to 40 mm for the magnet cuboid on the distal and from 10 mm to 45 mm for the magnetometer on the proximal. For the transverse distances, only the magnet cuboid was moved in 2.5 mm transverse steps within the 0 mm to 7.5 mm range. While positioned at each location within the test windows, the stepper motor generated quasi-static step rotations with an increment of 0.9° across two different ranges of motion: −18° to 110° for the FG and −75° to 75° for the WG. At each step, the motor paused for 1.5 seconds, and data of the magnetometer were collected. Curve fitting was performed on the collected data using third-order polynomials with Matlab R2022b (MathWorks), as the relationship must be bijective for practical use. Heat maps (FIG. 19B) illustrate the R² values from the curve fitting; 120 functions with R² values close to 0.99 were selected as reference functions and utilized in the calibration step.

### Measuring Changes in Joint Angles Using 3D Marker Tracking and 2D Markerless Tracking

A custom multi-camera setup was built to record videos of targeted joints undergoing specified movements (FIG. 31A). A previous study highlighted the advantages of this vision-based setup for analyzing the underlying mechanics in skin-interfaced electronics. DeepLabCut and Pose3D were used together to benchmark the device by estimating changes in joint angles. Although DeepLabCut functions as a markerless system, the tracked points were trained using physical markers to enhance accuracy—similar to marker—based systems such as Vicon and OptiTrack. Videos of flex/ext motions of PIP joints in all hand digits and both flex/ext and rad/uln motions of a wrist were captured using four time-synchronized high-speed cameras (2048×1088 resolution; HT-2000M, Emergent) equipped with 35 mm lenses (F1.4 manual focus; Kowa) at 60 fps. A user wore the electrogoniometry systems, which were equipped with four fiducial markers for this 3D tracking. The 3D tracking process involved stereo camera calibration, data collection, iterative frame labeling, network training, evaluation for all camera pairs, triangulation, and post-processing (FIG. 31B).

3D movements of fingers and wrists were associated with translations of up to 40 mm and rotations of up to 110°, leading to challenges in accurately estimating 3D positions over time. To address these challenges, over 100 stereo camera calibration images were collected and filtered based on a reprojection error of ε̅<0.2 pixels. Each dataset was then labeled and processed separately using a convolutional neural network (ResNet50), achieving a detection likelihood of over 90%. An open-source toolbox, Pose3D, was utilized for 3D reconstruction of the 2D tracked data. This approach achieved a root mean square deviation (RMSE) of less than 1 degree (FIG. 31D). The same technique without the 3D reconstruction process was applied to analyze videos recorded using a mini C-arm fluoroscopy (Fluoroscan Insight FD, Hologic Inc.) at 30 fps. DLC successfully recognized and tracked four key points—the head and base of the middle phalanx, and the head and center of the proximal phalanx—in these 2D fluoroscopic videos. Using the obtained positions of these points in 2D space, joint angles were accurately estimated.

### Protocols for Human Subject Studies

Adults between 18 to 65 years of age took part in the study. Although participants self-reported their sex, age and sex/gender were not considered in the human trials, as the study focused on joint angle measurements in the fingers. These measurements provide more relevant information about device functionality across varying hand sizes, which are more pertinent to hand therapy than age or sex/gender. No other factors associated with age or sex were expected to influence the study outcomes. Details of participants are available in Table 5.

What is claimed is:

1. A wireless wearable goniometer system comprising:
a. a wearable electrogoniometer sensor configured to detect changes in joint angles in a subject, comprising:
i. a magnet attached to a first appendage segment distal to a joint;
ii. a magnetometer attached to a second appendage segment proximal to the joint, wherein the magnetometer is configured to detect changes in a magnetic field induced by movements of the magnet associated with joint rotations; and
iii. a circuit board operatively connected to the magnetometer and comprising a power source, a power management integrated circuit (PMIC), and a sensor wireless data communication circuit; and
b. a computing device comprising:
i. a wireless data communication circuit configured to receive;
ii. an input device configured to receive user commands using a graphical user interface (GUI);
iii. a display; and
iv. at least one processor configured to:
1. receive signals from the wearable electrogoniometer sensor;
2. transform the signals from the wearable electrogoniometer sensor into the changes in joint angles in the subject; and
3. display the changes in joint angles in the subject on the display using the graphical user interface (GUI).

2. The wireless wearable goniometer system of claim 1, wherein the wearable electrogoniometer sensor further comprises an attachment means to secure the wearable electrogoniometer sensor to the subject, the attachment means selected from an adhesive, at least one strap with a pin-and-hole design, and any combination thereof.

3. The wireless wearable goniometer system of claim 1, wherein the wearable electrogoniometer sensor is encased in a silicone shell.

4. The wireless wearable goniometer system of claim 3, wherein the silicon shell has a thickness of 0.5 mm.

5. The wireless wearable goniometer system of claim 3, wherein the silicone shell comprises at least two fiducial markers.

6. The wireless wearable goniometer system of claim 5, wherein the at least two fiducial markers comprise a pattern of silicone with white and black dye, and further comprising a first fiducial marker located above the magnet, and a second fiducial marker located above the magnetometer.

7. The wireless wearable goniometer system of claim 1, wherein the power source comprises a 3.7-V lithium polymer battery

8. The wireless wearable goniometer system of claim 1, wherein the circuit board further comprises a folded charging coil for wireless charging.

9. The wireless wearable goniometer system of claim 1, wherein the magnet comprises a stack of four rectangular neodymium magnets.

10. The wireless wearable goniometer system of claim 1, wherein the wearable electrogoniometer sensor is configured to measure the joint angle in a joint selected from a finger joint, a wrist joint, and an elbow joint.

11. The wireless wearable goniometer system of claim 1, wherein the wireless data communication circuit comprises a Bluetooth module.

12. The wireless wearable goniometer system of claim 1, wherein the computing device is selected from a Smart phone, a tablet, and a laptop.

13. The wireless wearable goniometer system of claim 1, wherein the wearable goniometer system is configured to measure changes in the joint angle during events comprising vigorous activity and daily tasks.
